(19) United States
(12) Patent Application Publication
(10) Pub. No.: US 2009/0124672 A1
(43) Pub. Date: May 14, 2009

(54) ALDH-2 INHIBITORS IN THE TREATMENT OF PSYCHIATRIC DISORDERS

(76) Inventors: Ivan Diamond, Berkeley, CA (US); David Overstreet, Chapel Hill, NC (US)

Correspondence Address: CV THERAPEUTICS, INC., 3172 PORTER DRIVE, PALO ALTO, CA, 94304, US

(21) Appl. No.: 12/266,288

(22) Filed: Nov. 6, 2008

Related U.S. Application Data
(60) Provisional application No. 60/985,911, filed on Nov. 6, 2007.

(51) Int. Cl. A61K 31/4245; A61P 25/24

(52) U.S. Cl. 514/364

(57) ABSTRACT

Disclosed are isoflavone derivatives having the structure of Formula I

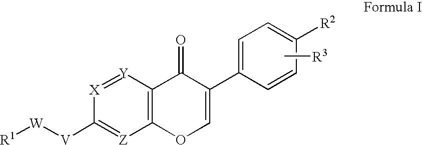

which are useful as ALDH-2 inhibitors for use treating in mammals suffering from psychiatric disorders such as, for example, depression, generalized anxiety, social phobia, panic disorder, and sleep disorders.

This application claims priority to U.S. Provisional Patent Application Ser. No. 60/985,911, filed Nov. 6, 2007, the entirety of which is incorporated herein by reference.

### FIELD OF THE INVENTION

The present invention relates to novel ALDH-2 inhibitors, and to their use in treating mammals for psychiatric disorders including but not limited to anxiety, depression, eating disorders, dementia, panic disorder, and sleep disorders.

### BACKGROUND

The isoflavone daidzin is the major active component obtained from extracts of Radix puerariae, a traditional Chinese medication that suppresses ethanol intake in Syrian golden hamsters. See Keung, W. M. and Vallee, B. L. (1993) Proc. Natl. Acad. Sci. USA 90, 10008-10012 and Keung, W. M., Klyosov, A. A., and Vallee, B. L. (1997) Proc. Natl. Acad. Sci. USA 94, 1675-1679, and U.S. Pat. Nos. 5,624,910 and 6,121,010. Removal of the sugar provides a compound known as daidzein, which has also been shown to be effective in suppressing ethanol uptake, but with decreased potency.

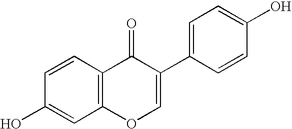

U.S. Pat. Nos. 5,624,910 and 6,121,010 disclosed ether derivatives of daidzin, which were shown to be effective in treating ethanol dependency. Daidzin and its analogs were shown to be potent and selective inhibitors of human mitochondrial aldehyde dehydrogenase (ALDH-2), which is an enzyme involved in the major enzymatic pathway responsible for ethanol metabolism in humans. It was also found that daidzin analogues that inhibit ALDH-2 but also inhibit the monamine oxidase (MAO) pathway were the least effective antidipsotropic activity.

In U.S. Patent Application Ser. No. 60/834,083, novel isoflavone derivatives were disclosed that are ALDH-2 inhibitors with little effect on the MAO pathway, and are useful for the treatment of alcohol dependency.

It has now surprisingly been found that ALDH-2 inhibitors are also useful for the treatment of various psychiatric disorders, including, but not limited to, anxiety, depression, eating disorders, dementia, panic disorder, and sleep disorders.

### SUMMARY OF THE INVENTION

Accordingly, in a first aspect, the invention relates to methods for treating psychiatric disorders including but not limited to depression, generalized anxiety, eating disorders, dementia, panic disorder, and sleep disorders by administration of a therapeutically effective amount of an ALDH-2 inhibitor.

In one embodiment of the invention, the ALDH-2 inhibitor is a compound of Formula I:

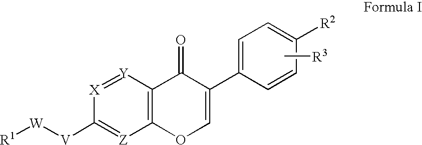

wherein:
R¹ is optionally substituted phenyl, optionally substituted heteroaryl, or optionally substituted heterocyclyl;
R² is hydrogen, hydroxy, halogen, optionally substituted lower alkoxy, optionally substituted lower alkyl, cyano, optionally substituted heteroaryl, C(O)OR⁵, —C(O)R⁵, —SO₂R¹⁵, —B(OH)₂, —OP(O)(OR⁵)₂, C(NR²⁰)NHR²², —NHR⁴, or —C(O)NHR⁵, in which,
R⁴ is hydrogen, —C(O)NHR⁵, or —SO₂R¹⁵, or —C(O)R⁵;
R⁵ is hydrogen, optionally substituted lower alkyl;
R¹⁵ is optionally substituted lower alkyl or optionally substituted phenyl; or
R² is —O-Q-R⁶, in which Q is a covalent bond or lower alkylene and R⁶ is optionally substituted heteroaryl;
R³ is hydrogen, cyano, optionally substituted amino, lower alkyl, lower alkoxy, or halo;
X, Y and Z are chosen from —CR⁷— and —N—, in which R⁷ is hydrogen, lower alkyl, lower alkoxy, or halo;
V is oxygen, sulfur, or —NH—; and
W is -Q¹-T-Q²-, wherein
Q¹ is a covalent bond or C₁₋₆ linear or branched alkylene optionally substituted with hydroxy, lower alkoxy, amino, cyano, or ═O;
Q² is C₁₋₆ linear or branched alkylene optionally substituted with hydroxy, lower alkoxy, amino, cyano, or ═O; and
T is a covalent bond, —O—, or —NH—, or
T and Q¹ may together form a covalent bond,
R²⁰ and R²² are independently selected from the group consisting of hydrogen, hydroxy, C₁₋₁₅ alkyl, C₂₋₁₅ alkenyl, C₂₋₁₅ alkynyl, heterocyclyl, aryl, benzyl, and heteroaryl,
wherein the alkyl, alkenyl, alkynyl, heterocyclyl, aryl, benzyl, and heteroaryl moieties are optionally substituted with from 1 to 3 substituents independently selected from halo, alkyl, mono- or dialkylamino, alkyl or aryl or heteroaryl amide, CN, O—C₁₋₆ alkyl, CF₃, OCF₃, B(OH)₂, Si(CH₃)₃, aryl, and heteroaryl.

In a second aspect of the invention, pharmaceutical formulations are provided comprising a therapeutically effective amount of an ALDH-2 inhibitor of Formula I, and at least one pharmaceutically acceptable carrier.

In one preferred embodiment, the invention relates to a group of compounds of Formula I in which X, Y and Z are all —CR⁶—, in which R⁶ is hydrogen. Within this group, preferred compounds include a class in which R¹ is optionally substituted phenyl, R² is 4-hydroxyl, R³ is hydrogen, V is oxygen, and W is methylene.

One preferred subclass within this class includes those compounds in which R¹ is phenyl substituted with from 1 to 3 substituents, which are independently selected from the group consisting of carboxyl, carboxylic ester, carboxamido, cyano, tetrazolyl, halo, or lower alkyl substituted by halo, particularly monosubstituted compounds in which the substitution is at the 3-position and disubstituted compounds in which the substitutions are at the 3,5-positions.

Another preferred class included compounds in which R¹ is optionally substituted phenyl, R² is 4-NHR⁴, R³ is hydrogen, V is oxygen, and W is methylene. One preferred subclass includes those compounds in which R¹ is phenyl substituted with from 1 to 3 substituents which are independently selected from the group consisting of carboxyl, carboxamido, cyano, tetrazolyl, halo, or lower alkyl substituted by halo, particularly monosubstituted compounds in which the substitution is at the 3-position and disubstituted compounds in which the substitutions are at the 3,5-positions. More preferred are those compounds where R⁴ is —SO₂R⁵, more preferably where R⁵ is methyl.

In another preferred group, R¹ is optionally substituted heteroaryl, particularly where R¹ is a five or six membered heteroaryl ring that includes oxygen and nitrogen atoms, V is oxygen, W is methylene, preferably where R² is 4-hydroxy and R³ is hydrogen. Within this group, one preferred subgroup includes those compounds in which R¹ is 1,3-oxazolyl, 1,3-thiazolyl, or (1,2,4-oxadiazol-3-yl), which are optionally substituted by phenyl substituted by carboxyl, carboxamido, cyano, tetrazolyl, halo, or lower alkyl substituted by halo, for example trifluoromethyl, particularly monosubstituted compounds in which the substitution is at the 3-position and disubstituted compounds in which the substitutions are at the 3,5-positions.

At present, the compounds for use in the invention include, but are not limited to:
3-{[3-(4-hydroxyphenyl)-4-oxochromen-7-yloxy]methyl}benzoic acid;
3-{[3-(4-hydroxyphenyl)-4-oxochromen-7-yloxy]methyl}benzenecarbonitrile;
3-(4-hydroxyphenyl)-7-[(3-(5H-1,2,3,4-tetrazol-5-yl)phenyl)methoxy]chromen-4-one;
3-{[3-(4-hydroxyphenyl)-4-oxochromen-7-yloxy]methyl}benzamide;
3-[(3-{4-[(methylsulfonyl)amino]phenyl}-4-oxochromen-7-yloxy)methyl]benzene-carbonitrile;
3-[(3-{4-[(methylsulfonyl)amino]phenyl}-4-oxochromen-7-yloxy)methyl]benzamide;
3-(4-hydroxyphenyl)-7-{[3-(trifluoromethyl)phenyl]methoxy}chromen-4-one;
3-(4-hydroxyphenyl)-7-{[4-methoxy-3-(trifluoromethyl)phenyl]methoxy}chromen-4-one;
7-{[3-fluoro-5-(trifluoromethyl)phenyl]methoxy}-3-(4-hydroxyphenyl)chromen-4-one;
3-(4-hydroxyphenyl)-7-{[5-(2-methoxyphenyl)(1,2,4-oxadiazol-3-yl)]methoxy}chromen-4-one;
3-(4-hydroxyphenyl)-7-[(5-phenyl(1,2,4-oxadiazol-3-yl))methoxy]chromen-4-one;
3-(4-hydroxyphenyl)-7-({5-[3-(trifluoromethyl)phenyl](1,2,4-oxadiazol-3-yl)}methoxy)chromen-4-one;
3-(4-hydroxyphenyl)-7-({5-[4-(trifluoromethyl)phenyl](1,2,4-oxadiazol-3-yl)}methoxy)chromen-4-one;
3-({5-[3-fluoro-5-(trifluoromethyl)phenyl](1,2,4-oxadiazol-3-yl)}methoxy)-3-(4-hydroxyphenyl)chromen-4-one;
3-({5-[4-fluoro-3-(trifluoromethyl)phenyl](1,2,4-oxadiazol-3-yl)}methoxy)-3-(4-hydroxyphenyl)chromen-4-one;
7-({5-[2,5-bis(trifluoromethyl)phenyl](1,2,4-oxadiazol-3-yl)}methoxy)-3-(4-hydroxyphenyl)chromen-4-one;
prop-2-enyl 3-(3-{[3-(4-hydroxyphenyl)-4-oxochromen-7-yloxy]methyl}-1,2,4-oxadiazol-5-yl)benzoate;
prop-2-enyl 3-{[3-(4-hydroxyphenyl)-4-oxochromen-7-yloxy]methyl}benzoate;
methyl 4-{[3-(4-hydroxyphenyl)-4-oxochromen-7-yloxy]methyl}benzoate;
methyl 3-{[3-(4-hydroxyphenyl)-4-oxochromen-7-yloxy]methyl}benzoate;
ethyl 4-{[3-(4-hydroxyphenyl)-4-oxochromen-7-yloxy]methyl}benzoate;
methylethyl 3-{[3-(4-hydroxyphenyl)-4-oxochromen-7-yloxy]methyl}benzoate;
4-{[3-(4-hydroxyphenyl)-4-oxochromen-7-yloxy]methyl}benzoic acid;
4-{[3-(4-hydroxyphenyl)-4-oxochromen-7-yloxy]methyl}benzamide;
3-(4-hydroxyphenyl)-7-{[5-(3-methoxyphenyl)(1,2,4-oxadiazol-3-yl)]methoxy}chromen-4-one; 3-(3-{[3-(4-hydroxyphenyl)-4-oxochromen-7-yloxy]methyl}-1,2,4-oxadiazol-5-yl)benzoic acid.
7-({5-[3,5-bis(trifluoromethyl)phenyl](1,2,4-oxadiazol-3-yl)}methoxy)-3-(4-hydroxyphenyl)chromen-4-one;
3-(3-{[3-(4-hydroxyphenyl)-4-oxochromen-7-yloxy]methyl}-1,2,4-oxadiazol-5-yl)benzenecarbonitrile;
3-(4-hydroxyphenyl)-7-[(3-phenyl(1,2,4-oxadiazol-5-yl))methoxy]chromen-4-one;
3-(4-hydroxyphenyl)-7-({3-[3-(trifluoromethyl)phenyl](1,2,4-oxadiazol-5-yl)}methoxy)chromen-4-one;
3-(4-hydroxyphenyl)-7-({3-[4-chlorophenyl](1,2,4-oxadiazol-5-yl)}methoxy)chromen-4-one;
3-(4-hydroxyphenyl)-2-(trifluoromethyl)-7-({5-[3-(trifluoromethyl)phenyl](1,2,4-oxadiazol-3-yl)}methoxy)chromen-4-one;
7-({5-[5-fluoro-3-(trifluoromethyl)phenyl](1,2,4-oxadiazol-3-yl)}methoxy)-3-(4-hydroxyphenyl)-2-(trifluoromethyl)chromen-4-one;
3-(4-hydroxyphenyl)-7-({5-[4-methoxy-3-(trifluoromethyl)phenyl](1,2,4-oxadiazol-3-yl)}methoxy)-2-(trifluoromethyl)chromen-4-one;
3-(4-hydroxyphenyl)-7-{[5-(3-(1H-1,2,3,4-tetraazol-5-yl)phenyl)(1,2,4-oxadiazol-3-yl)]methoxy}chromen-4-one;
3-(3-{[3-(4-hydroxyphenyl)-4-oxochromen-7-yloxy]methyl}-1,2,4-oxadiazol-5-yl)benzoic acid;
3-[(3-{4-[(methylsulfonyl)amino]phenyl}-4-oxochromen-7-yloxy)methyl]benzoic acid;
3-{4-[(methylsulfonyl)amino]phenyl}-7-({5-[3-(trifluoromethyl)phenyl](1,2,4-oxadiazol-3-yl)}methoxy)chromen-4-one;
7-{[5-(3-fluorophenyl)(1,2,4-oxadiazol-3-yl)]methoxy}-3-(4-hydroxyphenyl)chromen-4-one;
3-{4-[(methylsulfonyl)amino]phenyl}-7-({2-[4-(trifluoromethyl)phenyl](1,3-thiazol-5-yl)}methoxy)chromen-4-one.
4-[7-({5-[3-fluoro-5-(trifluoromethyl)phenyl](1,2,4-oxadiazol-3-yl)}methoxy)-4-oxochromen-3-yl]benzenecarbonitrile;
ethyl 4-[7-({4-methyl-2-[4-(trifluoromethyl)phenyl](1,3-thiazol-5-yl)}methoxy)-4-oxochromen-3-yl]benzoate;
7-({3-[5-fluoro-3-(trifluoromethyl)phenyl](1,2,4-oxadiazol-5-yl)}ethoxy)-3-(4-hydroxyphenyl)chromen-4-one;
ethyl 3-[7-({5-[3-fluoro-5-(trifluoromethyl)phenyl](1,2,4-oxadiazol-3-yl)}methoxy)-4-oxochromen-3-yl]benzoate;
3-{4-[(methylsulfonyl)amino]phenyl}-7-({4-methyl-2-[4-(trifluoromethyl)phenyl](1,3-thiazol-5-yl)}methoxy)chromen-4-one;
methyl 4-[7-({4-methyl-2-[4-(trifluoromethyl)phenyl](1,3-thiazol-5-yl)}methoxy)-4-oxochromen-3-yl]benzoate;
3-(2H,3H-benzo[e]1,4-dioxan-6-yl)-7-({5-[3-fluoro-5-(trifluoromethyl)phenyl](1,2,4-oxadiazol-3-yl)}methoxy)chromen-4-one;
7-({5-[3-fluoro-5-(trifluoromethyl)phenyl](1,2,4-oxadiazol-3-yl)}methoxy)-3-(6-methoxy(3-pyridyl))chromen-4-one;
3-(4-hydroxyphenyl)-7-({4-methyl-2-[4-(trifluoromethyl)phenyl](1,3-thiazol-5-yl)}methoxy)chromen-4-one;
7-({5-[3-fluoro-5-(trifluoromethyl)phenyl](1,2,4-oxadiazol-3-yl)}methoxy)-3-(4-{[(4-methylphenyl)sulfonyl]amino}phenyl)chromen-4-one;
3-(4-{[(4-methylphenyl)sulfonyl]amino}phenyl)-7-({4-methyl-2-[4-(trifluoromethyl)phenyl](1,3-thiazol-5-yl)}methoxy)chromen-4-one;
methyl 3-{[3-(6-methoxy(3-pyridyl))-4-oxochromen-7-yloxy]methyl}benzoate;
methyl 3-({3-[4-(hydroxymethyl)phenyl]-4-oxochromen-7-yloxy}methyl)benzoate;
7-({5-[3-fluoro-5-(trifluoromethyl)phenyl](1,2,4-oxadiazol-3-yl)}methoxy)-3-[4-(hydroxymethyl)phenyl]chromen-4-one;
4-[7-({5-[3-fluoro-5-(trifluoromethyl)phenyl](1,2,4-oxadiazol-3-yl)}methoxy)-4-oxochromen-3-yl]benzoic acid;
7-({5-[3-fluoro-5-(trifluoromethyl)phenyl](1,2,4-oxadiazol-3-yl)}methoxy)-3-(4-morpholin-4-ylphenyl)chromen-4-one;
7-({5-methyl-2-[4-(trifluoromethyl)phenyl](1,3-thiazol-4-yl)}methoxy)-3-(4-morpholin-4-ylphenyl)chromen-4-one;
7-({3-[5-fluoro-3-(trifluoromethyl)phenyl](1,2,4-oxadiazol-5-yl)}methoxy)-3-{4-[(methylsulfonyl)amino]phenyl}chromen-4-one;
2-fluoro-5-[7-({5-[5-fluoro-3-(trifluoromethyl)phenyl](1,2,4-oxadiazol-3-yl)}methoxy)-4-oxochromen-3-yl]benzenecarbonitrile;
ethyl 2-(3-{4-[(ethoxycarbonyl)methoxy]phenyl}-4-oxochromen-7-yloxy)acetate;
7-{[5-(4-fluorophenyl)(1,2,4-oxadiazol-3-yl)]methoxy}-3-(4-hydroxyphenyl)chromen-4-one;
3-[7-({5-[5-fluoro-3-(trifluoromethyl)phenyl](1,2,4-oxadiazol-3-yl)}methoxy)-4-oxochromen-3-yl]benzenecarbonitrile;
3-(3-acetylphenyl)-7-({5-[5-fluoro-3-(trifluoromethyl)phenyl](1,2,4-oxadiazol-3-yl)}methoxy)chromen-4-one;
7-({5-[5-fluoro-3-(trifluoromethyl)phenyl](1,2,4-oxadiazol-3-yl)}methoxy)-3-{4-[(methylsulfonyl)amino]phenyl}chromen-4-one;
4-[7-({5-[5-fluoro-3-(trifluoromethyl)phenyl](1,2,4-oxadiazol-3-yl)}methoxy)-4-oxochromen-3-yl]benzamide;
3-[2,4-bis(tert-butoxy)pyrimidin-5-yl]-7-({5-[5-fluoro-3-(trifluoromethyl)phenyl](1,2,4-oxadiazol-3-yl)}methoxy)chromen-4-one;
3-[7-({5-[5-fluoro-3-(trifluoromethyl)phenyl](1,2,4-oxadiazol-3-yl)}methoxy)-4-oxochromen-3-yl]-1,3-dihydropyrimidine-2,4-dione;
7-({2-[5-fluoro-3-(trifluoromethyl)phenyl]-(1,3-oxazol-4-yl)}methoxy)-3-(4-hydroxyphenyl)chromen-4-one;
3-(4-hydroxyphenyl)-7-({2-[3-(trifluoromethyl)phenyl](1,3-oxazol-4-yl)}methoxy)chromen-4-one;
7-({2-[5-fluoro-3-(trifluoromethyl)phenyl](1,3-oxazol-4-yl)}methoxy)-3-(4-hydroxyphenyl)chromen-4-one;
3-(4-hydroxyphenyl)-7-{[2-(3,4,5-trifluorophenyl)(1,3-oxazol-4-yl)]methoxy}chromen-4-one;
3-{[2-(3,5-difluorophenyl)(1,3-oxazol-4-yl)]methoxy}-3-(4-hydroxyphenyl)chromen-4-one;
3-{[2-(3,4-difluorophenyl)(1,3-oxazol-4-yl)]methoxy}-3-(4-hydroxyphenyl)chromen-4-one;
3-{[2-(4-fluorophenyl)(1,3-oxazol-4-yl)]methoxy}-3-(4-hydroxyphenyl)chromen-4-one;
7-{[2-(4-chlorophenyl)(1,3-oxazol-4-yl)]methoxy}-3-(4-hydroxyphenyl)chromen-4-one;
methyl 3-{[3-(4-hydroxyphenyl)-4-oxochromen-7-yloxy]methyl}benzoate;
3-(4-hydroxyphenyl)-7-({3-[3-(trifluoromethyl)phenyl](1,2,4-oxadiazol-5-yl)}methoxy)chromen-4-one;
3-(4-hydroxyphenyl)-2-(trifluoromethyl)-7-({5-[3-(trifluoromethyl)phenyl]-(1,2,4-oxadiazol-3-yl)}methoxy)chromen-4-one;
3-{[3-(4-hydroxyphenyl)-4-oxochromen-7-yloxy]methyl}benzenecarbonitrile;
3-(4-hydroxyphenyl)-7-({5-[3-(trifluoromethyl)phenyl]isoxazol-3-yl}methoxy)chromen-4-one;
7-{[5-(trifluoromethyl)(3-pyridyl)]methoxy}-3-(4-{[6-(trifluoromethyl)(3-pyridyl)]methoxy}phenyl)chromen-4-one;
3-(4-hydroxyphenyl)-7-[(5-(3-pyridyl)(1,2,4-oxadiazol-3-yl))methoxy]chromen-4-one;
3-(4-hydroxyphenyl)-7-[(5-(2-pyridyl)(1,2,4-oxadiazol-3-yl))methoxy]chromen-4-one;
methyl 2-{[3-(4-hydroxyphenyl)-4-oxochromen-7-yloxy]methyl}-1,3-oxazole-5-carboxylate;
3-{[5-(4-fluorophenyl)(1,2,4-oxadiazol-3-yl)]methoxy}-3-{4-[(methylsulfonyl)amino]-phenyl}chromen-4-one;
2-{[3-(4-hydroxyphenyl)-4-oxochromen-7-yloxy]methyl}-1,3-oxazole-5-carboxylic acid;
methyl 3-({3-[4-((1Z)-1-amino-2-methoxy-2-azavinyl)phenyl]-4-oxochromen-7-yloxy}methyl)benzoate;
3-{2-[4-(4-chlorophenyl)pyrazolyl]ethoxy}-3-(4-hydroxyphenyl)chromen-4-one;
3-(4-hydroxyphenyl)-7-[(6-pyrazolyl(3-pyridyl))methoxy]chromen-4-one;
3-[(2R)-2-hydroxy-3-({[3-(trifluoromethyl)phenyl]methyl}amino)propoxy]-3-(4-hydroxyphenyl)chromen-4-one;
3-(4-hydroxyphenyl)-7-[({[3-(trifluoromethyl)phenyl]methyl}amino)methoxy]chromen-4-one;
3-((2R)-3-{[(3,5-difluorophenyl)methyl]amino}-2-hydroxypropoxy)-3-(4-hydroxyphenyl)chromen-4-one;
3-(3-{[(1R)-1-(4-fluorophenyl)ethyl]amino}-2-oxopropoxy)-3-(4-hydroxyphenyl)chromen-4-one;
3-(4-hydroxyphenyl)-7-(3-phenylpropoxy)chromen-4-one;
3-{[5-(3-fluorophenyl)(1,3,4-oxadiazol-2-yl)]methoxy}-3-(4-hydroxyphenyl)chromen-4-one;
3-(4-hydroxyphenyl)-7-{[3-(trifluoromethyl)phenyl]ethoxy}chromen-4-one;
3-(4-hydroxyphenyl)-7-({5-[3-(trifluoromethyl)phenyl](1,3,4-oxadiazol-2-yl)}methoxy)chromen-4-one;
3-(4-hydroxyphenyl)-7-[(2-phenyl(1,3-oxazol-5-yl))methoxy]chromen-4-one;
7-({5-[3,5-bis(trifluoromethyl)phenyl]isoxazol-3-yl}methoxy)-3-(4-hydroxyphenyl)chromen-4-one;
3-(4-hydroxyphenyl)-7-({5-[3-(trifluoromethyl)phenyl]isoxazol-3-yl}methoxy)chromen-4-one;
3-{4-[(methylsulfonyl)amino]phenyl}-7-[(2-phenyl(1,3-oxazol-4-yl))methoxy]chromen-4-one;
2-[3-(4-hydroxyphenyl)-4-oxochromen-7-yloxy]-N-[3-(trifluoromethyl)phenyl]-acetamide;
7-{[5-(2-chlorophenyl)(1,3,4-thiadiazol-2-yl)]methoxy}-3-(4-hydroxyphenyl)chromen-4-one;
4-[7-({4-methyl-2-[4-(trifluoromethyl)phenyl](1,3-thiazol-5-yl)}methoxy)-4-oxochromen-3-yl]benzenecarbonitrile;
3-{4-[(methylsulfonyl)amino]phenyl}-7-({4-methyl-2-[4-(trifluoromethyl)phenyl](1,3-thiazol-5-yl)}methoxy)chromen-4-one;
3-(6-methoxy(3-pyridyl))-7-({4-methyl-2-[4-(trifluoromethyl)phenyl](1,3-thiazol-5-yl)}methoxy)chromen-4-one;
4-[7-({5-[5-fluoro-3-(trifluoromethyl)phenyl](1,3,4-oxadiazol-2-yl)}methoxy)-4-oxochromen-3-yl]benzenecarbonitrile;
4-[4-oxo-7-({3-[3-(trifluoromethyl)phenyl]isoxazol-5-yl}methoxy)chromen-3-yl]benzenecarbonitrile;
7-({5-[3-fluoro-5-(trifluoromethyl)phenyl](1,2,4-oxadiazol-3-yl)}methoxy)-3-{4-[(methylsulfonyl)amino]phenyl}chromen-4-one;
7-({5-[3-fluoro-5-(trifluoromethyl)phenyl](1,2,4-oxadiazol-3-yl)}methoxy)-3-[4-(methylsulfonyl)phenyl]chromen-4-one;
4-[7-({5-[3-fluoro-5-(trifluoromethyl)phenyl](1,2,4-oxadiazol-3-yl)}methoxy)-4-oxochromen-3-yl]benzamide;
3-(3-acetylphenyl)-7-({5-[3-fluoro-5-(trifluoromethyl)phenyl](1,2,4-oxadiazol-3-yl)}methoxy)chromen-4-one;
7-({5-[3-fluoro-5-(trifluoromethyl)phenyl](1,3,4-oxadiazol-2-yl)}methoxy)-3-(4-hydroxyphenyl)chromen-4-one;
7-({5-[3-fluoro-5-(trifluoromethyl)phenyl](1,2,4-oxadiazol-3-yl)}methoxy)-3-(5-hydropyrazol-4-yl)chromen-4-one;
ethyl 3-[7-({3-[3-fluoro-5-(trifluoromethyl)phenyl](1,2,4-oxadiazol-5-yl)}ethoxy)-4-oxochromen-3-yl]benzoate;
3-(4-hydroxyphenyl)-7-({2-[4-(trifluoromethyl)phenyl](1,3-thiazol-5-yl)}methoxy)chromen-4-one;
7-[2-(3-fluorophenyl)-2-oxoethoxy]-3-(4-hydroxyphenyl)chromen-4-one;
7-({5-[3-fluoro-5-(trifluoromethyl)phenyl](1,2,4-oxadiazol-3-yl)}ethoxy)-3-(4-hydroxyphenyl)chromen-4-one;
7-({5-[3-fluoro-5-(trifluoromethyl)phenyl](1,2,4-oxadiazol-3-yl)}methoxy)-3-(4-{[(4-methylphenyl)sulfonyl]amino}phenyl)chromen-4-one;
7-{[5-(2-chlorophenyl)(1,3,4-oxadiazol-2-yl)]methoxy}-3-(4-hydroxyphenyl)chromen-4-one;
7-{[5-(4-fluorophenyl)(1,3,4-oxadiazol-2-yl)]methoxy}-3-(4-hydroxyphenyl)chromen-4-one;
3-(4-hydroxyphenyl)-7-(4-pyridylmethoxy)chromen-4-one;
3-{4-[(methylsulfonyl)amino]phenyl}-7-({2-[4-(trifluoromethyl)phenyl](1,3-thiazol-5-yl)}methoxy)chromen-4-one;
2-[3-(4-hydroxyphenyl)-4-oxochromen-7-yloxy]-N-[2-(trifluoromethyl)phenyl]-acetamide;
3-(4-hydroxyphenyl)-7-{2-oxo-2-[2-(trifluoromethyl)phenyl]ethoxy}chromen-4-one;
3-(1H-indazol-5-yl)-7-({5-[5-fluoro-3-(trifluoromethyl)phenyl](1,2,4-oxadiazol-3-yl)}methoxy)chromen-4-one;
3-(4-hydroxyphenyl)-7-(2-phenylethoxy)chromen-4-one;
2-[3-(4-hydroxyphenyl)-4-oxochromen-7-yloxy]ethanenitrile;
7-[2-(4-chlorophenoxy)ethoxy]-3-(4-hydroxyphenyl)chromen-4-one;
3-{4-[7-({5-[5-fluoro-3-(trifluoromethyl)phenyl](1,2,4-oxadiazol-3-yl)}methoxy)-4-oxochromen-3-yl]phenyl}-1,3,5,6-tetrahydropyrimidine-2,4-dione;
N-[(1R)-1-(4-fluorophenyl)ethyl]-2-[3-(4-hydroxyphenyl)-4-oxochromen-7-yloxy]acetamide;
3-(4-hydroxyphenyl)-7-(2-pyridylmethoxy)chromen-4-one;
2-fluoro-5-[7-({5-[5-fluoro-3-(trifluoromethyl)phenyl](1,2,4-oxadiazol-3-yl)}methoxy)-4-oxochromen-3-yl]benzenecarbonitrile;
7-(2-pyridylmethoxy)-3-[4-(2-pyridylmethoxy)phenyl]chromen-4-one;
3-(4-hydroxyphenyl)-7-[(5-(4-pyridyl)(1,2,4-oxadiazol-3-yl))ethoxy]chromen-4-one;
3-(4-hydroxyphenyl)-7-[(5-(3-pyridyl)(1,2,4-oxadiazol-3-yl))ethoxy]chromen-4-one;
3-(4-hydroxyphenyl)-7-[(5-(2-pyridyl)(1,2,4-oxadiazol-3-yl))ethoxy]chromen-4-one;
3-(4-hydroxyphenyl)-7-{[5-(trifluoromethyl)(3-pyridyl)]methoxy}chromen-4-one;
7-{[5-(4-chlorophenyl)isoxazol-3-yl]methoxy}-3-(4-hydroxyphenyl)chromen-4-one;
7-{[5-(3,4-dichlorophenyl)isoxazol-3-yl]methoxy}-3-(4-hydroxyphenyl)chromen-4-one;
7-{[5-(4-chlorophenyl)isoxazol-3-yl]methoxy}-3-(4-hydroxyphenyl)chromen-4-one;
7-[(2R)-2-hydroxy-3-({[3-(trifluoromethyl)phenyl]methyl}amino)propoxy]-3-(4-hydroxyphenyl)chromen-4-one;
3-(4-hydroxyphenyl)-7-[2-({[3-(trifluoromethyl)phenyl]methyl}amino)ethoxy]chromen-4-one;
7-((2R)-3-{[(3,5-difluorophenyl)methyl]amino}-2-hydroxypropoxy)-3-(4-hydroxyphenyl)chromen-4-one;
methyl 2-{[3-(4-hydroxyphenyl)-4-oxochromen-7-yloxy]methyl}-1,3-oxazole-4-carboxylate;
2-{[3-(4-hydroxyphenyl)-4-oxochromen-7-yloxy]methyl}-1,3-oxazole-4-carboxylic acid;
N-[(1S)-1-(4-fluorophenyl)ethyl]-2-[3-(4-hydroxyphenyl)-4-oxochromen-7-yloxy]acetamide;
3-{[5-(4-fluorophenyl)(1,2,4-oxadiazol-3-yl)]methoxy}-3-(4-hydroxyphenyl)chromen-4-one;
3-{[5-(4-fluorophenyl)(1,2,4-oxadiazol-3-yl)]methoxy}-3-{4-[(methylsulfonyl)amino]-phenyl}chromen-4-one;
3-{3-[4-(4-chlorophenyl)pyrazolyl]propoxy}-3-(4-hydroxyphenyl)chromen-4-one;
3-(4-hydroxyphenyl)-7-(3-phenylpropoxy)chromen-4-one;
3-(4-hydroxyphenyl)-7-[(6-pyrazolyl(3-pyridyl))methoxy]chromen-4-one;
3-((2R)-2-hydroxy-3-phenylpropoxy)-3-(4-hydroxyphenyl)chromen-4-one;
3-(4-hydroxyphenyl)-7-[(5-(3-pyridyl)(1,3,4-oxadiazol-2-yl))methoxy]chromen-4-one;
3-[(2-hydroxy-3-{4-[(methylsulfonyl)amino]phenyl}-4-oxochromen-7-yloxy)methyl]benzoic acid;
3-{[5-(4-fluorophenyl)(1,3,4-oxadiazol-2-yl)]ethoxy}-3-(4-hydroxyphenyl)chromen-4-one;
3-(4-hydroxyphenyl)-7-[(5-(3-pyridyl)(1,3,4-oxadiazol-2-yl))ethoxy]chromen-4-one;
3-(4-hydroxyphenyl)-7-[(3-(3-pyridyl)(1,2,4-oxadiazol-5-yl))methoxy]chromen-4-one;
3-(4-hydroxyphenyl)-7-[(5-(3-pyridyl)(1,3,4-oxadiazol-2-yl))ethoxy]chromen-4-one;
3-(4-hydroxyphenyl)-7-[(5-(4-pyridyl)(1,2,4-oxadiazol-3-yl))ethoxy]chromen-4-one;
(2-{[3-(4-hydroxyphenyl)-4-oxochromen-7-yloxy]methyl}(1,3-oxazol-4-yl))-N-methylcarboxamide;
4-{[3-(4-hydroxyphenyl)-4-oxochromen-7-yloxy]methyl}-7-methoxychromen-2-one;
3-{[5-(4-fluorophenyl)(1,3,4-oxadiazol-2-yl)]methoxy}-3-{4-[(methylsulfonyl)amino]-phenyl}chromen-4-one;
7-{[5-(3-aminophenyl)(1,3,4-oxadiazol-2-yl)]methoxy}-3-(4-hydroxyphenyl)chromen-4-one;
ethyl 1-{2-[3-(4-hydroxyphenyl)-4-oxochromen-7-yloxy]ethyl}pyrazole-4-carboxylate;
3-{2-[4-(3-chlorophenyl)piperazinyl]ethoxy}-3-(4-hydroxyphenyl)chromen-4-one;
3-(4-hydroxyphenyl)-7-(2-{4-[3-(trifluoromethyl)phenyl]piperazinyl}ethoxy)chromen-4-one;
3-(4-hydroxyphenyl)-7-[(5-(2-pyridyl)isoxazol-3-yl)methoxy]chromen-4-one;
7-({3-[3-fluoro-5-(trifluoromethyl)phenyl](1,2,4-oxadiazol-5-yl)}ethoxy)-3-(4-hydroxyphenyl)chromen-4-one;
7-[2-(4-fluorophenyl)ethoxy]-3-(4-hydroxyphenyl)chromen-4-one;
7-((1R)-1-{3-[3-fluoro-5-(trifluoromethyl)phenyl](1,2,4-oxadiazol-5-yl)}ethoxy)-3-(4-hydroxyphenyl)chromen-4-one;
3-((1S)-1-{3-[3-fluoro-5-(trifluoromethyl)phenyl](1,2,4-oxadiazol-5-yl)}ethoxy)-3-(4-hydroxyphenyl)chromen-4-one;
3-(4-hydroxyphenyl)-7-{2-[3-(trifluoromethyl)pyrazolyl]ethoxy}chromen-4-one;
7-(1-{3-[3-fluoro-5-(trifluoromethyl)phenyl](1,2,4-oxadiazol-5-yl)}-isopropoxy)-3-(4-hydroxyphenyl)chromen-4-one;
3-(4-hydroxyphenyl)-7-[(3-(1H-1,2,3,4-tetraazol-5-yl)phenyl)methoxy]chromen-4-one;
prop-2-enyl 3-{[3-(4-aminophenyl)-4-oxochromen-7-yloxy]methyl}benzoate
3-(4-aminophenyl)-7-({5-[3-(trifluoromethyl)phenyl](1,2,4-oxadiazol-3-yl)}methoxy)chromen-4-one;
methyl 3-{[3-(4-aminophenyl)-4-oxochromen-7-yloxy]methyl}benzoate;
7-({5-[5-fluoro-3-(trifluoromethyl)phenyl](1,2,4-oxadiazol-3-yl)}methoxy)-3-(4-aminophenyl)chromen-4-one;)
3-{[3-(4-aminophenyl)-4-oxochromen-7-yloxy]methyl}benzenecarbonitrile;
3-{[3-(4-aminophenyl)-4-oxochromen-7-yloxy]methyl}benzamide;
prop-2-enyl 3-[(3-{4-[(methylsulfonyl)amino]phenyl}-4-oxochromen-7-yloxy)methyl]benzoate
methyl 3-[(3-{4-[(methylsulfonyl)amino]phenyl}-4-oxochromen-7-yloxy)methyl]benzoate;
7-({5-[5-fluoro-3-(trifluoromethyl)phenyl](1,2,4-oxadiazol-3-yl)}methoxy)-3-{4-[(methylsulfonyl)amino]phenyl}chromen-4-one;
3-[(3-{4-[(methylsulfonyl)amino]phenyl}-4-oxochromen-7-yloxy)methyl]-benzenecarbonitrile;
3-{[3-(4-methylsulfonylaminophenyl)-4-oxochromen-7-yloxy]methyl}benzamide;
3-{[3-(4-hydroxyphenyl)-4-oxochromen-7-yloxy]methyl}benzoic acid;
3-(3-{[3-(4-hydroxyphenyl)-4-oxochromen-7-yloxy]methyl}-1,2,4-oxadiazol-5-yl)benzoic acid;
methyl 3-({3-[4-(acetylamino)phenyl]-4-oxochromen-7-yloxy}methyl)benzoate;
3-(4-hydroxyphenyl)-7-{2-[4-(4-methoxyphenyl)piperazinyl]ethoxy}chromen-4-one;
7-{2-[4-(4-fluorophenyl)piperazinyl]ethoxy}-3-(4-hydroxyphenyl)chromen-4-one;
3-(4-hydroxyphenyl)-7-(2-piperazinylethoxy)chromen-4-one;
N-(3-fluorophenyl)(4-{2-[3-(4-hydroxyphenyl)-4-oxochromen-7-yloxy]ethyl}-piperazinyl)carboxamide;
3-[2-(4-{[(3-fluorophenyl)amino]thioxomethyl}piperazinyl)ethoxy]-3-(4-hydroxyphenyl)chromen-4-one;
N-(2,4-difluorophenyl)(4-{2-[3-(4-hydroxyphenyl)-4-oxochromen-7-yloxy]ethyl}piperazinyl)carboxamide;
3-(2-{2-[3-fluoro-5-(trifluoromethyl)phenyl](1,3-oxazol-5-yl)}ethoxy)-3-(4-hydroxyphenyl)chromen-4-one;
3-(3-{2-[3-fluoro-5-(trifluoromethyl)phenyl](1,3-oxazol-4-yl)}propoxy)-3-(4-hydroxyphenyl)chromen-4-one;
7-[2-(4-fluorophenyl)-2-oxoethoxy]-3-(4-hydroxyphenyl)chromen-4-one;
7-[2-(3-fluorophenyl)-2-oxoethoxy]-3-(4-hydroxyphenyl)chromen-4-one;
3-(4-hydroxyphenyl)-7-{2-oxo-2-[2-(trifluoromethyl)phenyl]ethoxy}chromen-4-one;
3-(4-hydroxyphenyl)-7-{2-oxo-2-[2-(trifluoromethyl)phenyl]ethoxy}chromen-4-one;
2-[3-(4-hydroxyphenyl)-4-oxochromen-7-yloxy]-N-[3-(trifluoromethyl)phenyl]-acetamide;
N-[(1S)-1-(4-fluorophenyl)ethyl]-2-[3-(4-hydroxyphenyl)-4-oxochromen-7-yloxy]acetamide;
2-[3-(4-hydroxyphenyl)-4-oxochromen-7-yloxy]-N-[2-(trifluoromethyl)phenyl]acetamide;
N-(3-fluorophenyl)-2-[3-(4-hydroxyphenyl)-4-oxochromen-7-yloxy]acetamide;
N-[(1R)-1-(4-fluorophenyl)ethyl]-2-[3-(4-hydroxyphenyl)-4-oxochromen-7-yloxy]acetamide;
3-(4-hydroxyphenyl)-7-[2-hydroxy-3-({[3-(trifluoromethyl)phenyl]methyl}amino)-propoxy]chromen-4-one;
(3-{[(3,5-difluorophenyl)methyl]amino}-2-hydroxypropoxy)-3-(4-hydroxyphenyl)chromen-4-one;
(2-{[(4-fluorophenyl)ethyl]amino}ethoxy)-3-(4-hydroxyphenyl)chromen-4-one;
3-(4-hydroxyphenyl)-7-(2-hydroxy-3-phenylpropoxy)chromen-4-one; and
7-((1R)-1-{3-[5-fluoro-3-(trifluoromethyl)phenyl](1,2,4-oxadiazol-5-yl)}ethoxy)-3-(4-hydroxyphenyl)chromen-4-one.

### BRIEF DESCRIPTION OF THE DRAWINGS

These and other features of the invention will be understood from the description of representative embodiments of the method herein and the disclosure of illustrative materials for carrying out the method, taken together with the Figures, wherein

FIG. 1 shows the effects of a compound of Formula I on social interaction in FH rats.

FIG. 2 shows the effects of a compound of Formula I on locomotor activity in FH rats.

FIG. 3 illustrates the effects of acute administration a compound of Formula I on social interaction after alcohol-withdrawal.

FIG. 4 is a graph of the effects of prophylactic treatment with a compound of Formula I on social interaction after alcohol-withdrawal.

FIG. 5 presents the effects of pretreatment with a compound of Formula I on line crosses by alcohol-withdrawn SD rats.

FIG. 6 shows the effects of a compound of Formula I on social interaction in rats subjected to restraint stress.

FIG. 7 illustrates the effects of a compound of Formula I on social interaction after treatment with DMCM, a benzodiazepine receptor inverse agonist.

FIG. 8 is a graph of the effects of a compound of Formula I on line crosses by SD rats treated with DMCM.

FIG. 9 presents the effects of a compound of Formula I on social interaction after treatment with mCPP, a 5-HT_{2C} agonist.

FIG. 10 shows the effects of a compound of Formula I on line crosses by SD Rats receiving mCPP.

### DETAILED DESCRIPTION

### Definitions and General Parameters

As used in the present specification, the following words and phrases are generally intended to have the meanings as set forth below, except to the extent that the context in which they are used indicates otherwise.

The term “alkyl” refers to a monoradical branched or unbranched saturated hydrocarbon chain having 1, 2, 3, 4, 5, 6, 7, 8, 9, 10, 11, 12, 13, 14, 15, 16, 17, 18, 19 or 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:
1) an alkyl group as defined above, having 1, 2, 3, 4 or 5 substituents, preferably 1 to 3 substituents, selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxyl, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
2) an alkyl group as defined above that is interrupted by 1-10 atoms independently chosen from oxygen, sulfur and NRₐ—, where Rₐ is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR, in which R is alkyl, aryl, or heteroaryl and n is 0, 1 or 2; or
3) an alkyl group as defined above that has both 1, 2, 3, 4 or 5 substituents as defined above and is also interrupted by 1-10 atoms as defined above.

The term “lower alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents, preferably 1, 2, or 3 substituents, as defined for substituted alkyl, or a lower alkyl group as defined above that is interrupted by 1, 2, 3, 4, or 5 atoms as defined for substituted alkyl, or a lower alkyl group as defined above that has both 1, 2, 3, 4 or 5 substituents as defined above and is also interrupted by 1, 2, 3, 4, or 5 atoms as defined above.

The term “alkylene” refers to a diradical of a branched or unbranched saturated hydrocarbon chain, having 1, 2, 3, 4, 5, 6, 7, 8, 9, 10, 11, 12, 13, 14, 15, 16, 17, 18, 19 or 20 carbon atoms, preferably 1-10 carbon atoms, more preferably 1, 2, 3, 4, 5 or 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “lower alkylene” refers to a diradical of a branched or unbranched saturated hydrocarbon chain, preferably having from 1, 2, 3, 4, 5, or 6 carbon atoms.

The term “lower alkylene” refers to a diradical of a branched or unbranched saturated hydrocarbon chain, preferably having from 1, 2, 3, 4, 5, or 6 carbon atoms.

The term “substituted alkylene” refers to:
(1) an alkylene group as defined above having 1, 2, 3, 4, or 5 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxyl, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2; or
(2) an alkylene group as defined above that is interrupted by 1-20atoms independently chosen from oxygen, sulfur and NFₐ—, where Rₐ is chosen from hydrogen, optionally substituted alkyl, cycloalkyl, cycloalkenyl, aryl, heteroaryl and heterocycyl, or groups selected from carbonyl, carboxyester, carboxyamide and sulfonyl; or
(3) an alkylene group as defined above that has both 1, 2, 3, 4 or 5 substituents as defined above and is also interrupted by 1-20 atoms as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers(—CH₂CH(CO₂H)CH₂—), ethoxyethyl (—CH₂CH₂O—CH₂CH₂—), ethylmethylaminoethyl (—CH₂CH₂N(CH₃)CH₂CH₂—),1-ethoxy-2-(2-ethoxy-ethoxy)ethane (—CH₂CH₂O—CH₂CH₂—OCH₂CH₂—OCH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “alkoxy” refers to the group R—O—, where R is optionally substituted alkyl or optionally substituted cycloalkyl, or R is a group —Y-Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Preferred alkoxy groups are optionally substituted alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like. The term “lower alkoxy” refers to the group R—O—, where R is optionally substituted lower alkyl as defined above.

The term “alkylthio” refers to the group R—S—, where R is as defined for alkoxy.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having 1-6, preferably 1, double bond (vinyl). Preferred alkenyl groups include ethenyl or vinyl (—CH═CH₂), 1-propylene or allyl (—CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “lower alkenyl” refers to alkenyl as defined above having from 2 to 6 carbon atoms.

The term “substituted alkenyl” refers to an alkenyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxyl, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl (or prop-1-yn-3-yl, —CH₂C≡CH), and the like. In the event that alkynyl is attached to nitrogen, the triple bond cannot be alpha to the nitrogen.

The term “substituted alkynyl” refers to an alkynyl group as defined above having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxyl, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aminocarbonyl” refers to the group —C(O)NRR where each R is independently hydrogen, alkyl, aryl, heteroaryl, heterocyclyl or where both R groups are joined to form a heterocyclic group (e.g., morpholino). Unless otherwise constrained by the definition, all substituents may optionally be further substituted by 1-3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “acylamino” refers to the group —NRC(O)R where each R is independently hydrogen, alkyl, aryl, heteroaryl, or heterocyclyl. Unless otherwise constrained by the definition, all substituents may optionally be further substituted by 1-3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “acyloxy” refers to the groups —O(O)C-alkyl, —O(O)C-cycloalkyl, —O(O)C-aryl, —O(O)C-heteroaryl, and —O(O)C-heterocyclyl. Unless otherwise constrained by the definition, all substituents may be optionally further substituted by alkyl, carboxyl, carboxyalkyl, aminocarbonyl, hydroxy, alkoxy, halogen, CF₃, amino, substituted amino, cyano, or —S(O)ₙR, where R is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl or anthryl). Preferred aryls include phenyl, naphthyl and the like.

The term “arylene” refers to a diradical of an aryl group as defined above. This term is exemplified by groups such as 1,4-phenylene, 1,3-phenylene, 1,2-phenylene, 1,4′-biphenylene, and the like.

Unless otherwise constrained by the definition for the aryl or arylene substituent, such aryl or arylene groups can optionally be substituted with from 1 to 5 substituents, preferably 1 to 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxyl, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “amino” refers to the group —NH₂.

The term “substituted amino” refers to the group —NRR where each R is independently selected from the group consisting of hydrogen, alkyl, cycloalkyl, carboxyalkyl (for example, benzyloxycarbonyl), aryl, heteroaryl and heterocyclyl provided that both R groups are not hydrogen, or a group —Y-Z, in which Y is optionally substituted alkylene and Z is alkenyl, cycloalkenyl, or alkynyl, Unless otherwise constrained by the definition, all substituents may optionally be further substituted by 1-3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “carboxyalkyl” refers to the groups —C(O)O-alkyl or —C(O)O-cycloalkyl, where alkyl and cycloalkyl, are as defined herein, and may be optionally further substituted by alkyl, alkenyl, alkynyl, alkoxy, halogen, CF₃, amino, substituted amino, cyano, or —S(O)ₙR, in which R is alkyl, aryl, or heteroaryl and n is 0, 1 or 2.

The term “cycloalkyl” refers to carbocyclic groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “substituted cycloalkyl” refers to cycloalkyl groups having 1, 2, 3, 4 or 5 substituents, and preferably 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxyl, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “halogen” or “halo” refers to fluoro, bromo, chloro, and iodo.

The term “acyl” denotes a group —C(O)R, in which R is hydrogen, optionally substituted alkyl, optionally substituted cycloalkyl, optionally substituted heterocyclyl, optionally substituted aryl, and optionally substituted heteroaryl.

The term “heteroaryl” refers to a radical derived from an aromatic cyclic group (i.e., fully unsaturated) having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring. Such heteroaryl groups can have a single ring (e.g., pyridyl or furyl) or multiple condensed rings (e.g., indolizinyl, benzothiazolyl, or benzothienyl). Examples of heteroaryls include, but are not limited to, [1,2,4]oxadiazole, [1,3,4]oxadiazole, [1,2,4]thiadiazole, [1,3,4]thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, thiazole, isothiazole, phenazine, oxazole, isoxazole, phenoxazine, phenothiazine, imidazolidine, imidazoline, and the like as well as N-oxide and N-alkoxy derivatives of nitrogen containing heteroaryl compounds, for example pyridine-N-oxide derivatives.

Unless otherwise constrained by the definition for the heteroaryl or heteroarylene substituent, such heteroaryl or heterarylene groups can be optionally substituted with 1 to substituents, preferably 1 to 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxyl, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “heteroaralkyl” refers to a heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 3-pyridylmethyl, quinolin-8-ylethyl, 4-methoxythiazol-2-ylpropyl, and the like.

The term “heteroaryloxy” refers to the group heteroaryl-O—.

The term “heterocyclyl” refers to a monoradical saturated or partially unsaturated group having a single ring or multiple condensed rings, having from 1 to 40 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholino, oxathiane, thiomorpholino, tetraydropthiophenyl, tetrahydropyranyl, tetrahydrothiopyranyl, piperidinyl, triazolidino, piperazinyl, dihydropyridino, pyrrolidinyl, imidazolidino, heyxahydropyrimidine, hezahydropyridazine, imidazoline, and the like.

Unless otherwise constrained by the definition for the heterocyclic substituent, such heterocyclic groups can be optionally substituted with 1, 2, 3, 4 or 5, and preferably 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxyl, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxyl, carboxyalkyl, aminocarbonyl, hydroxy, alkoxy, halogen, CF₃, amino, substituted amino, cyano, and —S(O)ₙR, where R is alkyl, aryl, or heteroaryl and n is 0, 1 or 2.

The term “thiol” refers to the group —SH.

The term “substituted alkylthio” refers to the group —S-substituted alkyl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, aryl, or heteroaryl. “Substituted sulfoxide” refers to a group —S(O)R, in which R is substituted alkyl, substituted aryl, or substituted heteroaryl, as defined herein.

The term “sulfone” refers to a group —S(O)₂R, in which R is alkyl, aryl, or heteroaryl. “Substituted sulfone” refers to a group —S(O)₂R, in which R is substituted alkyl, substituted aryl, or substituted heteroaryl, as defined herein.

The term “keto” refers to a group —C(O)—.

The term “thiocarbonyl” refers to a group —C(S)—.

The term “carboxyl” refers to a group —C(O)—OH.

“Optional” or “optionally” means that the subsequently described event or circumstance may or may not occur, and that the description includes instances where said event or circumstance occurs and instances in which it does not.

The term “compound of Formula I” is intended to encompass the compounds of the invention as disclosed, and the pharmaceutically acceptable salts, pharmaceutically acceptable esters, prodrugs, hydrates and polymorphs of such compounds. Additionally, the compounds of the invention may possess one or more asymmetric centers, and can be produced as a racemic mixture or as individual enantiomers or diastereoisomers. The number of stereoisomers present in any given compound of Formula I depends upon the number of asymmetric centers present (there are 2ⁿ stereoisomers possible where n is the number of asymmetric centers). The individual stereoisomers may be obtained by resolving a racemic or non-racemic mixture of an intermediate at some appropriate stage of the synthesis, or by resolution of the compound of Formula I by conventional means. The individual stereoisomers (including individual enantiomers and diastereoisomers) as well as racemic and non-racemic mixtures of stereoisomers are encompassed within the scope of the present invention, all of which are intended to be depicted by the structures of this specification unless otherwise specifically indicated.

“Isomers” are different compounds that have the same molecular formula.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(O)” is used to designate a racemic mixture where appropriate.

“Diastereoisomers” are stereoisomers that have at least two asymmetric atoms, but which are not mirror-images of each other.

The absolute stereochemistry is specified according to the Cahn-Ingold-Prelog R—S system. When the compound is a pure enantiomer the stereochemistry at each chiral carbon may be specified by either R or S. Resolved compounds whose absolute configuration is unknown are designated (+) or (−) depending on the direction (dextro- or laevorotary) which they rotate the plane of polarized light at the wavelength of the sodium D line.

“Parenteral administration” is the systemic delivery of the therapeutic agent via injection to the patient.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, and the age, physical condition, existence of other disease states, and nutritional status of the patient. Additionally, other medication the patient may be receiving will effect the determination of the therapeutically effective amount of the therapeutic agent to administer.

The term “treatment” or “treating” means any treatment of a disease in a mammal, including:
(i) preventing the disease, that is, causing the clinical symptoms of the disease not to develop;
(ii) inhibiting the disease, that is, arresting the development of clinical symptoms; and/or
(iii) relieving the disease, that is, causing the regression of clinical symptoms.

In many cases, the compounds of this invention are capable of forming acid and/or base salts by virtue of the presence of amino and/or carboxyl groups or groups similar thereto. The term “pharmaceutically acceptable salt” refers to salts that retain the biological effectiveness and properties of the compounds of Formula I, and which are not biologically or otherwise undesirable. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Nomenclature

The naming and numbering of the compounds of the invention is illustrated with a representative compound of Formula I in which R¹ is 5-[3-fluoro-5-(trifluoromethyl)phenyl]-(1,2,4-oxadiazol-3-yl) and R² is hydroxyl:

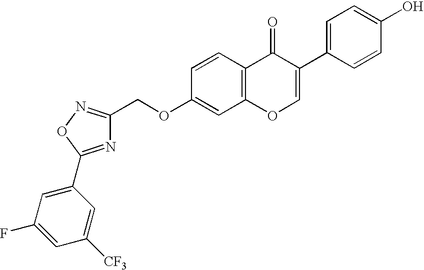

is named 7-({5-[3-fluoro-5-(trifluoromethyl)phenyl](1,2,4-oxadiazol-3-yl)}methoxy)-3-(4-hydroxyphenyl)chromen-4-one.

### Synthetic Reaction Parameters

The terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents.

The term “q.s.” means adding a quantity sufficient to achieve a stated function, e.g., to bring a solution to the desired volume (i.e., 100%).

### Synthesis of the Compounds of Formula I

The compounds of Formula I in which R² is hydroxy and X, Y and Z are all —CR⁶—, in which R⁶ is hydrogen may be prepared as shown in Reaction Scheme I.

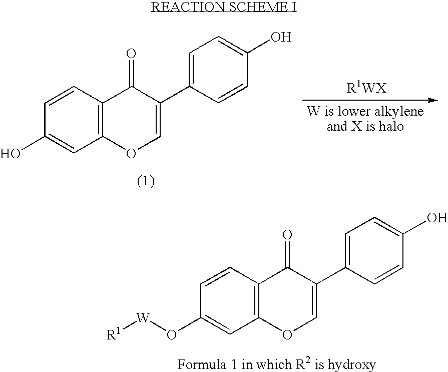

In general, the compound of formula (1), (daidzein, commercially available) is dissolved in an inert solvent, for example N,N-dimethylformamide, and reacted with about an equimolar amount of a compound of formula R¹WX, where W is lower alkylene of 1-3 carbon atoms and X is iodo, bromo or chloro, in the presence of a base, for example potassium carbonate, cesium carbonate, or the like. The reaction may be conducted at a temperature of about 50-100° C., for about 1-10 hours or may also be conducted at room temperature for 3 to 24 hours. When the reaction is substantially complete, the product of Formula I in which R² is hydroxy is isolated by conventional means, for example by precipitating the product out of solution by addition of water.

Alternatively, the compound of formula (1) is dissolved in an inert solvent, for example acetone, and an aqueous base added, for example 2N potassium hydroxide, and the mixture sonicated for about 5-30 minutes. The mixture is then reacted with about an equimolar amount of a compound of formula R¹WX, where W is lower alkyene of 1-3 carbon atoms and X is iodo, bromo or chloro, in the presence of about an equimolar amount of potassium iodide, and the mixture reacted at about reflux temperature for about 1-5 days. When the reaction is substantially complete, the product of Formula I in which R² is hydroxy is isolated by conventional means, for example by chromatography.

A method for preparing compounds of Formula I in which R¹ is phenyl substituted by tetrazol-5-yl, W is methylene, and X, Y and Z are all —CR⁶—, in which R⁶ is hydrogen is shown in Reaction Scheme II.

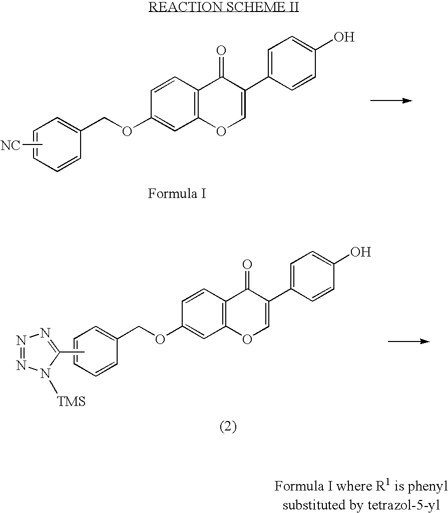

### Step 1—Preparation of a Compound of Formula (2)

In general, a mixture of the compound of Formula I in which R¹ is benzonitrile, dibutyltin oxide, and azidotrimethylsilane is subjected to microwaves. The reaction is conducted at a temperature of about 150° C. for about 10-30 minutes. When the reaction is substantially complete, the product of formula (2) is isolated by conventional means, for example by chromatography on silica gel.

### Step 2—Preparation of a Compound of Formula I

The purified product of formula (2) is suspended in an aqueous solvent, for example acetonitrile/water, and a catalytic amount of a strong acid added, for example trifluoroacetic acid. Removal of the solvents provides the compound of Formula I in which R¹ is phenyl substituted by tetrazol-5-yl.

Similarly, the compound of Formula I in which R¹ is [1,2,4]-oxadiazol-3-yl substituted by benzonitrile at the 5-position is converted to a compound of Formula I in which R¹ is [1,2,4]-oxadiazol-3-yl substituted by tetrazol-5-ylphenyl.

Compounds of Formula I in which R² is —NHR⁵ in which R⁵ is hydrogen may be prepared from an intermediate having a nitro group precursor, as shown in Reaction Scheme III.

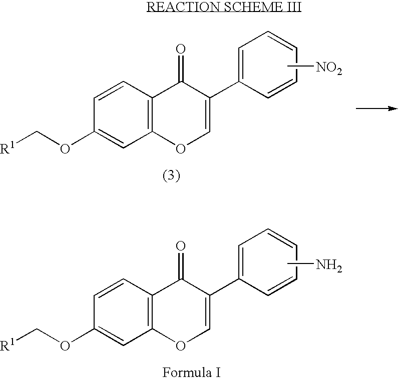

### Step 1—Preparation of a Compound of Formula I

In general, a nitro derivative of formula (3) (commercially available) is suspended in an aqueous solvent, for example a mixture of tetrahydrofuran and water, and reacted with sodium dithionite. The reaction is conducted at a temperature of about 50-70° C. overnight. When the reaction is substantially complete, the amine of Formula I is isolated by conventional means, for example by chromatography on silica gel.

It should be noted that if the compound of formula (3) has a carboxyl group present on the R¹ moiety, the carboxyl group is better protected as an allyl ester before carrying out the reduction of the nitro group. Such a protecting group protects the carboxyl group in any subsequent reaction in which the amine is, for example acylated, and is easily removed after acylation, whereas an alkyl ester is more difficult to hydrolyze under conventional hydrolysis conditions.

Conversion of a compound of Formula I in which W is methylene, X, Y and Z are all —CR⁶—, in which R⁶ is hydrogen, and R² is NH₂ to a corresponding compound of Formula I in which R² is NHSO₂R⁵ is shown in Reaction Scheme IV.

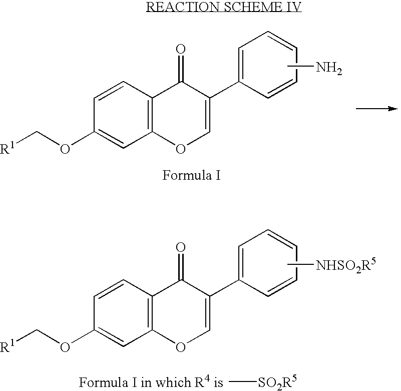

In general, the compound of Formula I in which R² is amino is suspended in an inert solvent, for example dichloromethane, and a tertiary base added, for example pyridine. The mixture is cooled to about 0° C., a compound of formula R⁵SO₂Cl added, and the mixture reacted for about 1-2 hours. When the reaction is substantially complete, the compound of Formula I in which R⁴ is —SO₂R⁵ is isolated by conventional means, for example by chromatography on silica gel.

Similarly, reaction of a compound of Formula I in which R² is amino with an acylating agent of formula ClC(O)R⁵ provides compounds of Formula I in which R² is —NHR⁴ where R⁴ is —C(O)R⁵. Reaction with a compound of formula ClC(O)NHR⁵ or R⁵NCO provides compounds of Formula I in which R⁴ is —C(O)NHR⁵.

When a carboxyl group present on the R¹ moiety has been protected as an allyl ester before carrying out the reduction of the nitro group, conversion of a compound of Formula I in which W is methylene, X, Y and Z are all —CR⁶—, in which R⁶ is hydrogen, and R¹ is an allyl ester derivative to a corresponding compound of Formula I in which R¹ is an acid derivative is shown in Reaction Scheme V.

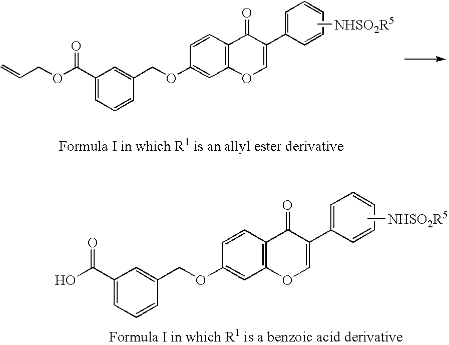

In general, an allyl ester derivative of Formula I is dissolved in an inert solvent, for example tetrahydrofuran, and a base, for example morpholine, and tetrakis(triphenyl-phosphine)palladium(0) added. The reaction is conducted at about room temperature for about 1-12 hours. When the reaction is substantially complete, the compound of Formula I in which R¹ is a benzoic acid derivative is isolated by conventional means, for example by flash chromatography on silica gel.

The compounds of formula R¹WCl are either commercially available, or are made by methods well known in the art. For example, to prepare compounds of Formula I in which R¹ is oxazole substituted with optionally substituted phenyl, the synthesis starts from a compound of formula (4) (which is a compound of formula R¹WCl in which R¹ is optionally substituted 1,3-oxazole and W is methylene), the preparation of which is shown in Reaction Scheme VI.

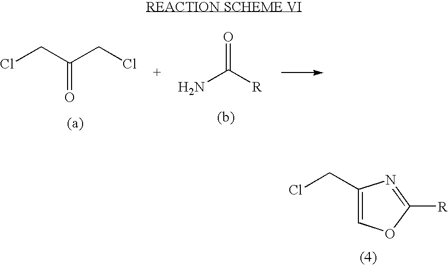

where R is optionally substituted phenyl.

In general, 1,3-dichloroacetone (a) is reacted with an appropriately substituted benzamide derivative of formula (b), in which R is optionally substituted phenyl. The reaction is conducted at a temperature of about 100-140° C., for about 1-6 hours. When the reaction is substantially complete, the compound of formula (4) is isolated by conventional means, for example by flash chromatography on silica gel or recrystallization from an inert solvent.

The compound of formula (4) is then reacted with a compound of formula (1), (daidzein, commercially available) as shown in Reaction Scheme I above, to provide a compound of Formula I.

Similarly, a compound of formula R¹WCl in which R¹ is optionally substituted 1,3,4-oxadiazole and W is methylene can be prepared as shown in Reaction Scheme VIA

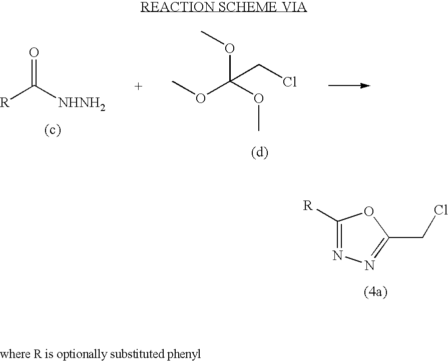

The hydrazide of formula (c), which is commercially available or made by means well known in the art, is suspended in 2-chlorotrimethoxyethane (d) in the presence of an organic acid, for example acetic acid. The mixture is carried out a temperature of about 140-180° C., in a microwave oven. When the reaction is substantially complete, the compound of formula (4a) is isolated by conventional means.

Similarly, a compound of formula R¹WCl in which R¹ is optionally substituted 1,2,4-oxadiazole and W is alkylene can be prepared as shown in Reaction Scheme VIB

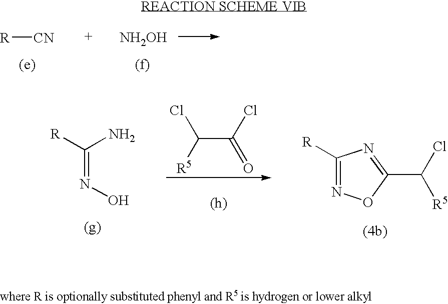

### Step 1

In general, the nitrile of formula (e), in which R is optionally substituted phenyl, is reacted with aqueous hydroxylamine (formula (f)) in a protic solvent, for example ethanol. The reaction is conducted at a temperature of about 50-100° C., for about 2 hours. When the reaction is substantially complete, the compound of formula (g) is isolated by conventional means.

### Step 2

The compound of formula (g) is then reacted with a compound of formula (h), in which R⁵ is hydrogen or lower alkyl. The reaction is conducted at a temperature of about 50-100° C., for about 2 hours. When the reaction is substantially complete, the compound of formula (4b) is isolated by conventional means.

The compound of formula (4b) is then reacted with a compound of formula (1), (daidzein, commercially available) as shown in Reaction Scheme I above, to provide a compound of Formula I.

Alternatively, a compound of formula R¹WCl in which R¹ is optionally substituted 1,2,4-oxadiazole and W is alkylene may also be prepared as shown in Reaction Scheme VIB′

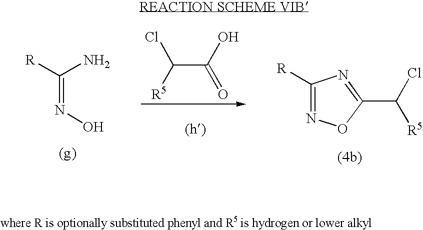

The compound of formula (g) is reacted with the compound of formula (h′), in which R⁵ is hydrogen or lower alkyl. The compound of formula (h′) is placed in as suitable solvent such a dichloromethane and cooled to approximately 0° C. After 20 to 40 minutes, the compound of formula (g′) is added and the coupling reaction allowed to proceed for 1 to 2 hours. CBr₄ and Ph₃P are then added and the dehydration allowed to proceed for an additional 4 to 6 hours. Solid triphenylphosine oxide is removed and the remaining solvent evaporated and the compound of formula (4b) is isolated by conventional means.

As before, the compound of formula (4b) is then reacted with a compound of formula (1), (daidzein, commercially available) as shown in Reaction Scheme I above, to provide a compound of Formula I.

Similarly, a compound of formula R¹WCl in which R¹ is isoxazole and W is

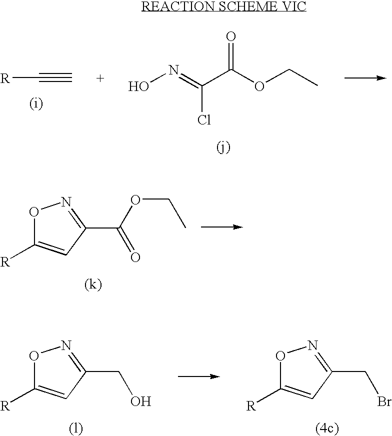

### Step 1

In general, the acetylene derivative of formula (i), in which R is optionally substituted phenyl, is reacted with ethyl chlorooximidoacetate (formula (j)) in an inert solvent, for example tetrahydrofuran, in the presence of a base, for example triethylamine. The reaction is conducted at a temperature of about 0-25° C., for about 10-24 hours. When the reaction is substantially complete, the compound of formula (k) is isolated by conventional means.

### Step 2

In general, the ester derivative of formula (k), in which R is optionally substituted phenyl, is reacted with a reducing agent, for example sodium borohydride in a protic solvent, for example ethanol. The reaction is initially conducted at a temperature of about 0° C., and then at room temperature for about 1-2 hours. When the reaction is substantially complete, the compound of formula (1) is isolated by conventional means.

### Step 3

In general, the hydroxymethyl derivative of formula (1), in which R is optionally substituted phenyl, is reacted with a brominating agent, for example carbon tetrabromide in the presence of triphenylphosphine. The reaction is conducted at a temperature of about 0° C. for about 1-2 hours. When the reaction is substantially complete, the compound of formula (4c) is isolated by conventional means.

An alternative method of preparing compounds of Formula I is shown in Reaction Scheme VII.

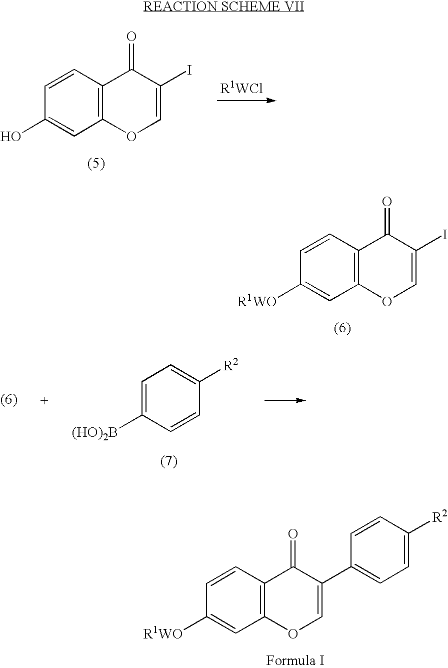

### Step 1

In general, the compound of formula (5), 7-hydroxy-3-iodochromen-4-one, is reacted with a compound of formula R¹WCl in a polar solvent, for example N,N-dimethylformamide, in the presence of sodium iodide and a mild base, for example potassium carbonate. The reaction is conducted at a temperature of about 40-80° C., for about 1 hour or may be conducted at room temperature for a longer period, 2 to 24 hours. When the reaction is substantially complete, the compound of formula (6) is isolated by conventional means, for example by flash chromatography on silica gel or

### Step 2

The compound of formula (6) is then reacted with the boronic acid of formula (7), which are either commercially available or prepared by means well known in the art. In general, the reaction is conducted in an inert solvent, for example dimethoxymethane, in the presence of tetrakistriphenylphosphine palladium and aqueous sodium carbonate. The reaction is conducted at a temperature of about 60-100° C., for about 1 hour. When the reaction is substantially complete, the compound of Formula I is isolated by conventional means, for example by flash chromatography on silica gel or recrystallization from an inert solvent.

As will be evident to one of ordinary skill in the art, the compound of formula (7) may first be reacted with the compound of formula (5) to produce a desired compound of formula (5a) as shown below:

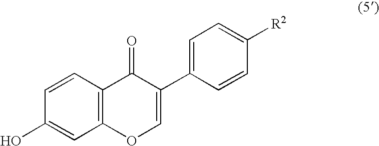

which may then be reacted with a compound of formula R¹WX as described above.

One method of preparing the starting material 3-iodo-7-methoxychromen-4-one is shown in Reaction Scheme VIII.

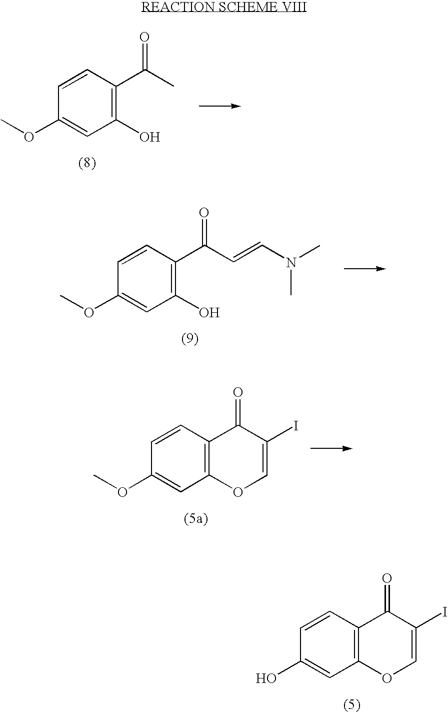

### Step 1

In general, the compound of formula (8), 1-(2-hydroxy-4-methoxyphenyl)ethan-1-one, is reacted with the dimethylacetal of N,N-dimethylformamide. The reaction is conducted at a temperature of about 50-100° C., for about 2 hours. When the reaction is substantially complete, the compound of formula (9) is isolated by conventional means, for example by filtration of the precipitated product, 3-(dimethylamino)-1-(2-hydroxy-4-methoxyphenyl)prop-2-en-1-one.

### Step 2

The compound of formula (9) is then reacted with N-iodosuccinimide in an inert solvent, for example chloroform, in the presence of silica gel. The reaction is conducted at a temperature of about 0° C., for about 1 hour. When the reaction is substantially complete, the compound of formula (5a), 3-iodo-7-methoxychromen-4-one, is isolated by conventional means, for example by filtering off the silica gel, washing the solid with chloroform, and removal of the solvent.

### Step 3

The compound of formula (5a) is then reacted with boron tribromide to convert the methoxy group to a hydroxyl group. In general, the compound of formula (5a) is dissolved in an inert solvent, for example chloroform, cooled to about −80° C., and reacted with boron tribromide for about 1 hour. The mixture is then allowed to warm to about room temperature, and stirred for about 2-5 days. When the reaction is substantially complete, the compound of formula (5), 3-iodo-7-hydroxychromen-4-one, is isolated by conventional means.

It will be appreciated by those of skill in the art that various Q¹ and Q² linking groups can be added to either the R¹WX reactant or the compound of formula (6) prior to the final synthesis of the compound of Formula I. Such alkylation techniques are well within the skill of one of ordinary skill in the art and will be readily apparent. Similarly, methods for subsequent modification of the R¹, R², or R³, substituent after the synthesis of a compound of Formula I will also be readily apparent to one of ordinary skill.

For example, a method of making compounds wherein Q¹ is methylene, T is NH, and Q² is ethylene is shown in Reaction Scheme IX:

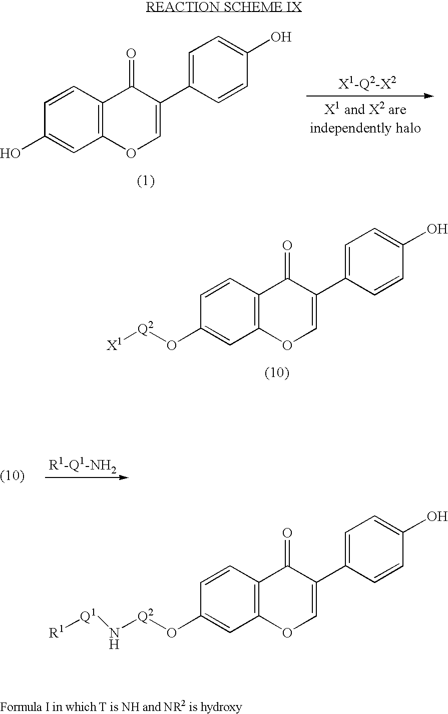

### Step 1

The commercially available compound of formula (1) is dissolved in an inert solvent, for example acetone, and an aqueous base added, for example 2N potassium hydroxide. The mixture is then reacted with about an equimolar amount of a compound of formula X¹Q²X², where X¹ and X² are independently iodo, bromo or chloro. The mixture is reacted at about reflux temperature for about 1-5 days. The solvent is then evaporated and the residue purified using conventional methods such as column chromatography to provide the compound of formula (10).

### Step 2

The compound of formula (10) is the reacted with a compound of formula R¹Q¹-NH₂ in an inert solvent such as DMF. The reaction takes place at a temperature of approximately 50° C. to 80° C. for 12 to 48 hours. When the reaction is substantially complete, the compound of Formula I is isolated by conventional means, for example by solvent evaporation followed by TLC.

As will be apparent to one of ordinary skill in the art, this type of reaction can be modified so that a modified Q¹ linking group is added to an appropriately halogenated R¹ derivative according the method described in Step 2 to provide a compound of the formula R¹-Q¹-X.

In another variation of the synthesis, oxirane derivatives of desired Q¹ and/or Q² linking groups may be used to produce compounds of Formula I wherein either or both of the Q moieties are hydroxy substituted. For example, a method of making compounds wherein Q¹ is methylene, T is NH, and Q² is 2-hydroxy propylene is shown in Reaction Scheme X:

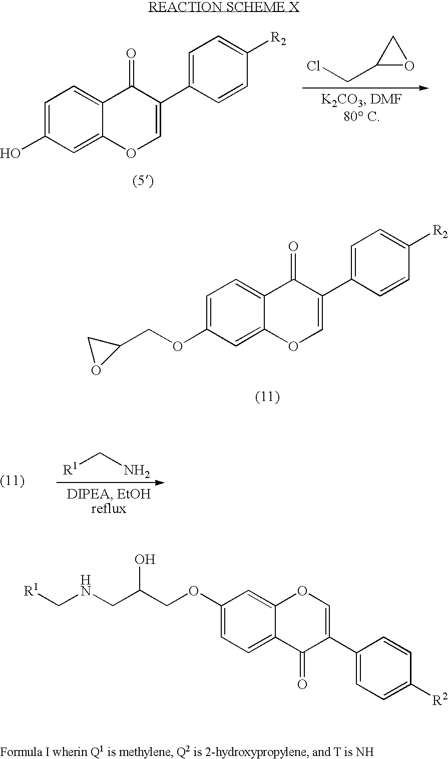

### Step 1

The compound of formula (5′) is reacted with epichlorohydrin and K₂CO₃ in a suitable solvent such as DMF. The reaction takes place at a temperature ranging from 60° C. to 90° C. and is carried out for 1 to 6 hours. When the reaction is substantially complete, the solvent is removed by evaporation and the compound of formula (11) collected as a precipitate from the residue by treatment with H₂O. The precipitate may be collected conventional means, for example by flash chromatography on silica gel or recrystallization from an inert solvent.

### Step 2

The compound of formula (11) is then reacted with an amino derivative of the desired R¹Q¹ segment, such as the R¹-methylamino compound shown in Reaction Scheme X. The reactants are dissolved in a protic solvent such as ethanol and a catalytic amount of base such as DIPEA (N,N′-diisopropylethylamine) is added. The reaction may be carried out by stirring overnight at temperature of 70° C. to 85° C. When the reaction is substantially complete, the solvent is removed by evaporation and the compound of Formula I collected and purified by conventional means such as silica gel column chromatography followed by recrystallization from an inert solvent.

In instances where compounds wherein T is a covalent bond, the compound of formula (11) can be reacted with a magnesium bromide derivative of the desired R¹Q¹ segment. In this type of reaction, the magnesium bromide derivative is slowly added to a cooled (−60° to −30° C.) solution of CuI in THF. To this solution is then slowly added the compound of formula (11) in THF. The reaction mixture is stirred at −60° to −30° C. 1 to 2 hours then quenched with saturated NH₄Cl aqueous solution and H₂O and extracted with EtOAc. The organic layer is further washed with brine, then dried over Na₂SO₄ and evaporated in vacuo. The compound of Formula I is then collected and purified by conventional means such as prep-TLC.

### Utility, Testing and Administration

### General Utility

The compounds of Formula I are generally effective in the treatment of conditions that respond to administration of ALDH-2 inhibitors. Specifically, the compounds of Formula I are useful in the treatment of psychiatric disorders including but not limited to depression, generalized anxiety, eating disorders, dementia, panic disorder, and sleep disorders.

While not wishing to be bound by theory, it is believed that ALDH-2 inhibitors are effective in treating psychiatric disorders as a consequence of their ability to normalize and/or modulate dopamine and serotonin levels. Inhibition of ALDH-2 has been shown to inhibit serotonin and dopamine metabolism and to increase the levels of various biogenic aldehydes related to these neurotransmitters, see Keung et al., (1998) Proc Natl Acad Sci USA. March 3; 95(5):2198-203. As modulation of dopamine and/or serotonin levels is a known method for the treatment of any number of psychiatric disorders, ALDH-2 inhibitors can be used to provide effective treatment for a wide variety of psychiatric disorders.

For purposes of illustration, the following definitions of depressive and anxiety disorders are taken from the Diagnostic and Statistical Manual of Mental Disorders. 4th ed. (DSM-IV; American Psychiatric Association, 1994a) or Diagnostic and Statistical Manual of Mental Disorders. 3rd ed. Revised (DSM-III-R; American Psychiatric Association, 1987). Depressive disorders include major depressive disorder and dysthymic disorder (American Psychiatric Association, 1994a; American Psychiatric Association, 1994b). Major depressive disorder is characterized by the occurrence of one or more major depressive episodes without manic or hypomanic episodes. A major depressive episode is defined as a prominent and relatively persistent depressed or dysphoric mood that usually interferes with daily functioning (nearly every day for at least 2 weeks); it should include at least 4 of the following 8 symptoms: change in appetite, change in sleep, psychomotor agitation or retardation, loss of interest in usual activities or decrease in sexual drive, increased fatigue, feelings of guilt or worthlessness, slowed thinking or impaired concentration, and a suicide attempt or suicidal ideation (Medical Economics Company, 2002). Dysthymic disorder involves a type of depression that is not severe enough to be called a major depressive episode, but that lasts much longer than major depressive disorder, without high phases.

It is contemplated that ALDH-2 inhibitors will be effective in treating depression in patients who have been diagnosed with depression by administration of any of the following tests: Hamilton Depression Rating Scale (HDRS), Hamilton depressed mood item, Clinical Global Impressions (CGI)-Severity of Illness. It is further contemplated that ALDH-2 inhibitors will be effective in inducing improvements in certain of the factors measured in these tests, such as the HDRS subfactor scores, including the depressed mood item, sleep disturbance factor and anxiety factor, and the CGI-Severity of Illness rating. It is also contemplated that ALDH-2 inhibitors will be effective in preventing relapse of major depressive episodes.

Anxiety disorders include panic disorder, agoraphobia with or without history of panic disorder, specific phobia, social phobia, obsessive-compulsive disorder, post-traumatic stress disorder, acute stress disorder and generalized anxiety disorder.

It is contemplated that ALDH-2 inhibitors will be effective in treating any or all of these disorders in patients who have been diagnosed with these disorders.

Panic disorder is characterized by recurrent unexpected panic attacks and associated concern about having additional attacks, worry about the implications or consequences of the attacks, and/or a significant change in behavior related to the attacks (American Psychiatric Association, 1994a). A panic attack is defined as a discrete period of intense fear or discomfort in which four (or more) of the following symptoms develop abruptly and reach a peak within 10 minutes: (1) palpitations, pounding heart, or accelerated heart rate; (2) sweating; (3) trembling or shaking; (4) sensations of shortness of breath or smothering; (5) feeling of choking; (6) chest pain or discomfort; (7) nausea or abdominal distress; (8) feeling dizzy, unsteady, lightheaded, or faint; (9) derealization (feelings of unreality) or depersonalization (being detached from oneself); fear of losing control; (11) fear of dying; (12) paresthesias (numbness or tingling sensations); (13) chills or hot flushes. Panic disorder may or may not be associated with agoraphobia, or an irrational and often disabling fear of being out in public. It is contemplated that ALDH-2 inhibitors will be effective in treating panic disorder in patients who have been diagnosed with panic disorder on the basis of frequency of occurrence of panic attacks, or by means of the CGI-Severity of Illness scale. It is further contemplated that the compounds of the invention will be effective in inducing improvements in certain of the factors measured in these evaluations, such as a reduction in frequency or elimination of panic attacks, an improvement in the CGI-Severity of Illness scale or a CGI-Global Improvement score of 1 (very much improved), 2 (much improved) or 3 (minimally improved). It is also contemplated that the compounds of this invention will be effective in preventing relapse of panic disorder.

Social anxiety disorder, also known as social phobia, is characterized by a marked and persistent fear of one or more social or performance situations in which the person is exposed to unfamiliar people or to possible scrutiny by others (American Psychiatric Association, 1994a). Exposure to the feared situation almost invariably provokes anxiety, which may approach the intensity of a panic attack. The feared situations are avoided or endured with intense anxiety or distress. The avoidance, anxious anticipation, or distress in the feared situation (s) interferes significantly with the person's normal routine, occupational or academic functioning, or social activities or relationships, or there is marked distress about having the phobias. Lesser degrees of performance anxiety or shyness generally do not require psychopharmacological treatment. It is contemplated that the compounds of this invention will be effective in treating social anxiety disorder in patients who have been diagnosed with social anxiety disorder by administration of any of the following tests: the Liebowitz Social Anxiety Scale (LSAS), the CGI-Severity of Illness scale, the Hamilton Rating Scale for Anxiety (HAM-A), the Hamilton Rating Scale for Depression (HAM-D), the axis V Social and Occupational Functioning Assessment Scale of DSM-IV, the axis II (ICD-10) World Health Organization Disability Assessment, Schedule 2 (DAS-2), the Sheehan Disability Scales, the Schneier Disability Profile, the World Health Organization Quality of Life-10 (WHOQOL-100), or other tests as described in Bobes, 1998 (J Clin Psychiatry. 1998; 59 Suppl 17:12-9), which is incorporated herein by reference. It is further contemplated that ALDH-2 inhibitors will be effective in inducing improvements as measured by these tests, such as the a change from baseline in the Liebowitz Social Anxiety Scale (LSAS), or a CGI—Global Improvement score of 1 (very much improved), 2 (much improved) or 3 (minimally improve d). It is also contemplated that the compounds of this invention will be effective in preventing relapse of social anxiety disorder.

Generalized anxiety disorder is characterized by excessive anxiety and worry (apprehensive expectation) that is persistent for at least 6 months and which the person finds difficult to control (American Psychiatric Association, 1994a). It must be associated with at least 3 of the following 6 symptoms: restlessness or feeling keyed up or on edge, being easily fatigued, difficulty concentrating or mind going blank, irritability, muscle tension, sleep disturbance. The diagnostic criteria for this disorder are described in further detail in DSM-IV, which is incorporated herein by reference (American Psychiatric Association, 1994a). It is contemplated that ALDH-2 inhibitors will be effective in treating generalized anxiety disorder in patients who have been diagnosed with this disorder according to the diagnostic criteria described in DSM-IV. It is further contemplated that the compounds of the invention will be effective in reducing symptoms of this disorder, such as the following: excessive worry and anxiety, difficulty controlling worry, restlessness or feeling keyed up or on edge, being easily fatigued, difficulty concentrating or mind going blank, irritability, muscle tension, or sleep disturbance. It is also contemplated that ALDH-2 inhibitors will be effective in preventing relapse of general anxiety disorder.

In a preferred embodiment, the subject invention provides a method of treatment or management of the following indications: depressive disorders and anxiety disorders. Examples of depressive disorders are major depressive disorder or dysthymic disorder. Examples of anxiety disorders are panic disorder, agoraphobia without history of panic disorder, specific phobia, social phobia, obsessive-compulsive disorder, post-traumatic stress disorder, acute stress disorder or generalized anxiety disorder.

### Testing

Activity testing is conducted as described in those patents and patent applications referenced above, and in the Examples below, and by methods apparent to one skilled in the art. For example, as described in “The Mitochondrial Monoamine Oxidase-Aldehyde Dehydrogenase Pathway: A Potential Site of Action of Daidzin”, J. Med. Chem. (2000) 43: 4169-4179. In general, the compounds of Formula I are assayed to determine their effects on MAO and ALDH-2 independently using the membrane and lysate of a density-gradient-purified mitochondria preparation as the respective enzyme sources. The results are expressed in IC50 values.

Methods of assessing the efficacy of ALDH-2 inhibitors in the treatment of psychiatric disorders are well-known in the art and will be readily apparent to those of skill in the art. For example, animal models for assessing antidepressant and/or anxiolytic effects include, but are not limited to, the Forced Swim Test, the Tail Suspension Test, the Defensive Burying Test, the Light/Dark Preference Test, the Maternal Separation Test, the Elevated Plus-Maze test, and the Light-Enhanced Startle test. Neurobiological tests include, but are not limited to, microdialysis to measure the effect of drug on noradrenaline, serotonin, and dopamine concentrations in target brain regions. Measurement of glucocorticoid receptor expression may also be used to assess efficacy and may be carried out via Northern blots, Western blots, RNAase protection, in situ hybridization, immunocytochemistry, and in vivo by assaying glucocorticoid receptor translocation and binding to nuclear DNA.

### Pharmaceutical Compositions

The compounds of Formula I are usually administered in the form of pharmaceutical compositions. This invention therefore provides pharmaceutical compositions that contain, as the active ingredient, one or more of the compounds of Formula I, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The compounds of Formula I may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G. S. Banker & C. T. Rhodes, Eds.).

### Administration

The compounds of Formula I may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parental, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of Formula I in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of the compounds of Formula I. Administration may be via capsule or enteric coated tablets, or the like. In making the pharmaceutical compositions that include at least one compound of Formula I, the active ingredient is usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds of Formula I are effective over a wide dosage range and is generally administered in a pharmaceutically effective amount. Preferably, for oral administration, each dosage unit contains from 10 mg to 2 g of a compound of Formula I, more preferably from 10 to 700 mg, and for parenteral administration, preferably from 10 to 700 mg of a compound of Formula I, more preferably about 50-200 mg. It will be understood, however, that the amount of the compound of Formula I actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Preparation of a Compound of Formula (4)

A. Preparation of a Compound of Formula (4) in which R is Phenyl

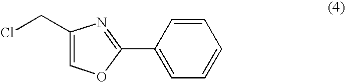

A 50 mL round bottomed flask equipped with a condenser was charged with benzamide (a compound of formula (b), 363.4 mg, 3.0 mmol) and 1,3-dichloroacetone (457.1 mg, 3.6 mmol, 1.2 equiv.). This mixture was heated at 130° C. for 1 hour under a nitrogen atmosphere. After cooling to room temperature, the resulting mixture was purified by recrystallization from acetonitrile (6 mL). The suspension was heated under reflux reaction condition for 5 minutes and cooled down to ambient temperature. The resulting solid was filtered through a glass filter, and the crystals on the filter were washed with acetonitrile (2 mL). The desired product, 4-(chloromethyl)-2-phenyl-1,3-oxazole, was obtained as a colorless powder (285.8 mg, 1.48 mmol, 49%).

B. Preparation of other Compounds of Formula (4a) in which R is Phenyl

Similarly, following the procedures of Example 1A, substituting other compounds of formula (b) for benzamide, other compounds of formula R¹WCl were prepared. For example:
4-(chloromethyl)-2-[5-fluoro-3-(trifluoromethyl)phenyl]-1,3-oxazole;
2-(3,5-difluorophenyl)-4-(chloromethyl)-1,3-oxazole;
2-(3,4-difluorophenyl)-4-(chloromethyl)-1,3-oxazole;
4-(chloromethyl)-2-(4-fluorophenyl)-1,3-oxazole;
4-(chloromethyl)-2-(4-chlorophenyl)-1,3-oxazole;
4-(chloromethyl)-2-[3-(trifluoromethyl)phenyl]-1,3-oxazole; and
4-(chloromethyl)-2-(3,4,5-trifluorophenyl)-1,3-oxazole.
   C. Preparation of a Compound of Formula (4a) in which R is 4-Fluorophenyl

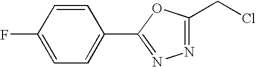

4-Fluorobenzenecarbohydrazide (0.3 g, 2 mmol) was suspended in chloro-1,1,1-trimethoxyethane (2 ml). To the suspension was added acetic acid (1 ml), and the solution was heated in a microwave for 30 minutes at 160° C. The solvent was removed under reduced pressure, and the residue purified using Biotage, eluting with 20% ethyl acetate/hexanes, to provide 5-(chloromethyl)-3-(4-fluorophenyl)-1,2,4-oxadiazole in 89% yield.

D. Preparation of a Compound of Formula (4b) in which R is 5-Fluoro-3-Trifluoromethylphenyl and R⁵ is Methyl

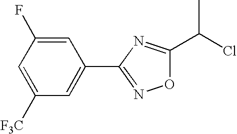

### Step 1

To a solution of 5-fluoro-3-(trifluoromethyl)benzenecarbonitrile (15.0 g, 79.3 mmol) in ethanol (30 ml) was added a solution of 50% hydroxylamine in water (10 ml, 151.5 mmol), and the resulting mixture was heated at 80° C. for 2 hours. The mixture was cooled to room temperature, solvent removed under reduced pressure, and 30 ml of water added. The suspension was sonicated and the solid filtered off, washed with water (2×20 ml), and dried under reduced pressure, to provide [5-fluoro-3-(trifluoromethyl)phenyl](hydroxyimino)methylamine as a white solid. MS 223.1 (M+H).

### Step 2

To a solution of [5-fluoro-3-(trifluoromethyl)phenyl](hydroxyimino)methylamine (8.884 g, 40 mmol) in a mixture of anhydrous dichloromethane/N,N-dimethylformamide (60/20 ml) was added 2-chloropropanoyl chloride (6.0 ml, 58.7 mmol) and diisopropylethylamine (14.0 ml, 80.3 mmol), and the mixture was stirred at room temperature for two hours. The mixture was then refluxed overnight with stirring, cooled to room temperature, and solvent removed under reduced pressure. The residue was fractionally distilled under vacuum, and the portion boiling at 95-105° C./0.8-1.0 mm Hg retained, to provide 5-(chloroethyl)-3-[5-fluoro-3-(trifluoromethyl)phenyl]-1,2,4-oxadiazole as a yellow oil, MS 295.1 (M+H).

Alternatively, the product can be purified by flash chromatography over silica gel, eluting with ethyl acetate/hexanes (1/4).

E. Preparation of a Compound of Formula (4c) in which R is 3-trifluoromethylphenyl

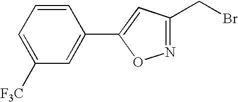

### Step 1—Preparation of a Compound of Formula (k)

To a stirred solution of ethyl chlorooximidoacetate (6.68 g, 44.09 mmol) in tetrahydrofuran (90 mL) in an ice bath was added 3-(trifluoromethyl)phenylacetylene (5.0 g, 29.39 mmol) slowly, followed by triethylamine (8.19 mL, 58.78 mmol) dropwise. The resulting mixture was stirred at room temperature overnight, which was then filtered through a layer of silica gel (top) and anhydrous Na₂SO₄ (bottom), and washed with ethyl acetate. The filtrate was washed with water, the organic layer dried over sodium sulfate, and the solvent removed under reduced pressure. The residue was purified by silica gel column chromatography (ethyl acetate:Hexanes=1:9) to afford ethyl 5-[3-(trifluoromethyl)phenyl]isoxazole-3-carboxylate.

Similarly prepared was ethyl 5-(2-pyridyl)isoxazole-3-carboxylate.

### Step 2—Preparation of a Compound of Formula (1)

To a stirred solution of ethyl 5-[3-(trifluoromethyl)phenyl]isoxazole-3-carboxylate (2 g, 7 mmol) in ethanol (70 mL) in an ice bath was added sodium borohydride (1.06 g, 28 mmol) portionwise. The resulting mixture was stirred at room temperature for 1.5 hours, which was then quenched with saturated ammonium chloride aqueous solution. Solvent was removed from the mixture under reduced pressure, and the residue was dissolved in ethyl acetate and washed with water. The organic layer was then dried over sodium sulfate, and solvent removed under reduced pressure. The residue was purified by silica gel column chromatography (ethyl acetate:Hexanes=2:3) to afford {5-[3-(trifluoromethyl)phenyl]isoxazol-3-yl}methan-1-ol.

Similarly prepared was (5-(2-pyridyl)isoxazol-3-yl)methan-1-ol.

### Step 3—Preparation of a Compound of Formula (4c)

To a stirred suspension of {5-[3-(trifluoromethyl)phenyl]isoxazol-3-yl}methan-1-ol (0.28 g, 1.15 mmol) and carbon tetrabromide (0.5 g, 1.5 mmol) in methylene chloride (10 mL) at 0° C. was added dropwise a solution of triphenylphosphine (0.41 g, 1.58 mmol) in methylene chloride (5 mL). The resulting mixture was stirred at 0° C. for 1 hour, then the reaction mixture poured into ethyl acetate and Hexanes (ethyl acetate:Hexanes=1:4, 50 mL). The resulting suspension was filtered through a thin layer of silica gel and washed with ethyl acetate and Hexanes (ethyl acetate:Hexanes=1:4). The filtrate was concentrated under reduced pressure, and the residue was purified by silica gel column chromatography (ethyl acetate:Hexanes=1:4) to afford 3-(bromomethyl)-5-[3-(trifluoromethyl)phenyl]isoxazole.

Similarly prepared was 3-(chloromethyl)-5-(2-pyridyl)isoxazole

### EXAMPLE 2

### Preparation of a Compound of Formula (5)

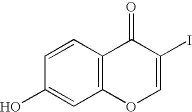

### Step 1—Preparation of a Compound of Formula (9)

A mixture of 1-(2-hydroxy-4-methoxyphenyl)ethan-1-one (20 g, 120 mmol) and N,N-dimethylformamide dimethylacetal (23 g, 181 mmol) was stirred at 90° C. for 2 hours. After cooling to room temperature the reaction mixture provided a yellow precipitate, which was washed with ethyl acetate (3×30 ml), water (2×50 ml), and dried under reduced pressure to yield 3-(dimethylamino)-1-(2-hydroxy-4-methoxyphenyl)prop-2-en-1-one (9) as the trans isomer; MS 222.1 (M+H)

### Step 2—Preparation of a Compound of Formula (5)

To a solution of 3-(dimethylamino)-1-(2-hydroxy-4-methoxyphenyl)prop-2-en-1-one (20.0 g, 90.37 mmol) in anhydrous chloroform (100 ml) at 0° C. was added N-iodosuccinimide (23.5 g, 99.22 mmol) and silica gel (40g). The reaction mixture was stirred at 0° C. for 60 minutes, then the insoluble material filtered off. The filtrate was washed with aqueous sodium thiosulfate (0.5M, 2×50 ml), followed by brine (100 ml), then dried over sodium sulfate. The solvent was removed under reduced pressure, providing an orange solid. To this solid was added methanol (30 ml), and the mixture was sonicated, filtered, the solid washed with methanol (2×5 ml), and the solid dried under reduced pressure, to give 3-iodo-7-methoxychromen-4-one as a pale yellow solid.

This product (9.36 g, 30.98 mmol) was dissolved in anhydrous chloroform (10 ml), and cooled to −78° C. To this solution was added a 1.0 M solution of boron tribromide in methylene chloride (90 ml, 90 mmol), and the mixture stirred for 1 hour at −78° C. The mixture was allowed to warm to room temperature, and stirred for 4 days. The mixture was then poured into water (200 ml), and the brown solid filtered off, washed with water (4×100 ml), and chloroform (3×20 ml). The filtrate was concentrated under reduced pressure to give a yellow gel, to which was added methylene chloride (20 ml), and the mixture sonicated. A pale yellow solid was obtained, and was filtered off, washed with methylene chloride (2×5 ml), and dried under reduced pressure to provide 7-hydroxy-3-iodochromen-4-one.

### EXAMPLE 3

### Preparation of a Compound of Formula I

Step 1. Preparation of a Compound of Formula (6) in which R¹ is 4-Methyl-2-[4-(trifluoromethyl)phenyl](1,3-thiazol-5-yl) and W is Methylene

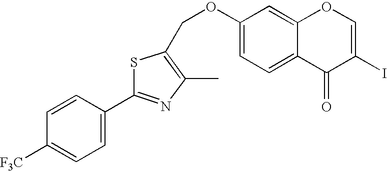

A mixture of 7-hydroxy-3-iodochromen-4-one (864 mg, 3.0 mmol), 5-(chloromethyl)-4-methyl-2-(4-(trifluoromethyl)phenyl)thiazole) (875 mg, 3.0 mmol), sodium iodide (450 mg, 3.0 mmol), and potassium carbonate (552 mg, 4.0 mmol) was dissolved in N,N-dimethylformamide (10 ml) at room temperature under nitrogen. The mixture was heated at 60° for 1 hour, cooled to room temperature, and water (30 ml) added to the mixture. The aqueous mixture was extracted with methylene chloride (3×30 ml), and the combined organic layer washed with brine (30 ml), dried over sodium sulfate, and solvent removed from the filtrate under reduced pressure. Crystallization of the crude product from ethyl acetate (4 ml) gave 3-iodo-7-({4-methyl-2-[4-(trifluoromethyl)phenyl](1,3-thiazol-5-yl)}methoxy)chromen-4-one, a compound of formula (6).

Step 2—Preparation of a Compound of Formula I in which R¹ is Phenyl](1,3-thiazol-5-yl) R² is 4-Methylsulfonamide R³ is Hydrogen, V is Oxygen, X, Y, and Z are —CH—, and W is Methylene

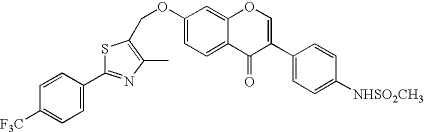

To a mixture of 3-iodo-7-({4-methyl-2-[4-(trifluoromethyl)phenyl](1,3-thiazol-5-yl)}methoxy)chromen-4-one (55.0 mg, 0.10 mmol), 4-(dihydroxyboron) (methylsulfonyl)phenylamine (methylsulfonyl)phenylamine (22.5 mg, 0.15 mmol), bis-(triphenylphosphine) palladium (TI) dichloride (3.5 mg, 0.005 mmol) was added dimethoxyethane (2 ml) and aqueous sodium carbonate solution (2M, 0.1 ml, 2 equivs). The mixture was refluxed for 1 hour, cooled to ambient temperature, filtered through celite (3 g), and the celite washed with ethyl acetate (50 ml). The filtrate was washed with brine (30 ml), and dried over sodium sulfate. The solvent was removed under reduced pressure, and the residue chromatographed on silica gel, eluting with ethyl acetate/hexanes 50/1, after which the product was crystallized from ethyl acetate (3 ml), to provide 3-{4-[(methylsulfonyl)amino]phenyl}-7-({2-[4-(trifluoromethyl)phenyl] (1,3-thiazol-5-yl)}methoxy)chromen-4-one.

### B.

Similarly, the following compounds of Formula I were prepared:
4-[7-({5-[3-fluoro-5-(trifluoromethyl)phenyl](1,2,4-oxadiazol-3-yl)}methoxy)-4-oxochromen-3-yl]benzenecarbonitrile;
ethyl 4-[7-({4-methyl-2-[4-(trifluoromethyl)phenyl](1,3-thiazol-5-yl)}methoxy)-4-oxochromen-3-yl]benzoate;
7-({3-[5-fluoro-3-(trifluoromethyl)phenyl](1,2,4-oxadiazol-5-yl)}ethoxy)-3-(4-hydroxyphenyl)chromen-4-one;
ethyl 3-[7-({5-[3-fluoro-5-(trifluoromethyl)phenyl](1,2,4-oxadiazol-3-yl)}methoxy)-4-oxochromen-3-yl]benzoate;
3-{4-[(methylsulfonyl)amino]phenyl}-7-({4-methyl-2-[4-(trifluoromethyl)phenyl](1,3-thiazol-5-yl)}methoxy)chromen-4-one;
methyl 4-[7-({4-methyl-2-[4-(trifluoromethyl)phenyl](1,3-thiazol-5-yl)}methoxy)-4-oxochromen-3-yl]benzoate;
3-(2H,3H-benzo[e]1,4-dioxan-6-yl)-7-({5-[3-fluoro-5-(trifluoromethyl)phenyl](1,2,4-oxadiazol-3-yl)}methoxy)chromen-4-one;
7-({5-[3-fluoro-5-(trifluoromethyl)phenyl](1,2,4-oxadiazol-3-yl)}methoxy)-3-(6-methoxy(3-pyridyl))chromen-4-one;
3-(4-hydroxyphenyl)-7-({4-methyl-2-[4-(trifluoromethyl)phenyl] (1,3-thiazol-5-yl)}methoxy)chromen-4-one;
7-({5-[3-fluoro-5-(trifluoromethyl)phenyl](1,2,4-oxadiazol-3-yl)}methoxy)-3-(4-{[(4-methylphenyl)sulfonyl]amino}phenyl)chromen-4-one;
3-(4-{[(4-methylphenyl)sulfonyl]amino}phenyl)-7-({4-methyl-2-[4-(trifluoromethyl)phenyl](1,3-thiazol-5-yl)}methoxy)chromen-4-one;
methyl 3-{[3-(6-methoxy(3-pyridyl))-4-oxochromen-7-yloxy]methyl}benzoate;
methyl 3-({3-[4-(hydroxymethyl)phenyl]-4-oxochromen-7-yloxy}methyl)benzoate;
7-({5-[3-fluoro-5-(trifluoromethyl)phenyl](1,2,4-oxadiazol-3-yl)}methoxy)-3-[4-(hydroxymethyl)phenyl]chromen-4-one;
4-[7-({5-[3-fluoro-5-(trifluoromethyl)phenyl](1,2,4-oxadiazol-3-yl)}methoxy)-4-oxochromen-3-yl]benzoic acid;
7-({5-[3-fluoro-5-(trifluoromethyl)phenyl](1,2,4-oxadiazol-3-yl)}methoxy)-3-(4-morpholin-4-ylphenyl)chromen-4-one;
7-({5-methyl-2-[4-(trifluoromethyl)phenyl] (1,3-thiazol-4-yl)}methoxy)-3-(4-morpholin-4-ylphenyl)chromen-4-one;
7-({3-[5-fluoro-3-(trifluoromethyl)phenyl](1,2,4-oxadiazol-5-yl)}methoxy)-3-{4-[(methylsulfonyl)amino]phenyl}chromen-4-one;
2-fluoro-5-[7-({5-[5-fluoro-3-(trifluoromethyl)phenyl](1,2,4-oxadiazol-3-yl)}methoxy)-4-oxochromen-3-yl]benzenecarbonitrile;
ethyl 2-(3-{4-[(ethoxycarbonyl)methoxy]phenyl}-4-oxochromen-7-yloxy)acetate;
7-{[5-(4-fluorophenyl)(1,2,4-oxadiazol-3-yl)]methoxy}-3-(4-hydroxyphenyl)chromen-4-one;
3-[7-({5-[5-fluoro-3-(trifluoromethyl)phenyl](1,2,4-oxadiazol-3-yl)}methoxy)-4-oxochromen-3-yl]benzenecarbonitrile;
3-(3-acetylphenyl)-7-({5-[5-fluoro-3-(trifluoromethyl)phenyl](1,2,4-oxadiazol-3-yl)}methoxy)chromen-4-one;
7-({5-[5-fluoro-3-(trifluoromethyl)phenyl](1,2,4-oxadiazol-3-yl)}methoxy)-3-{4-[(methylsulfonyl)amino]phenyl}chromen-4-one;
4-[7-({5-[5-fluoro-3-(trifluoromethyl)phenyl](1,2,4-oxadiazol-3-yl)}methoxy)-4-oxochromen-3-yl]benzamide;
3-[2,4-bis(tert-butoxy)pyrimidin-5-yl]-7-({5-[5-fluoro-3-(trifluoromethyl)phenyl](1,2,4-oxadiazol-3-yl)}methoxy)chromen-4-one; and
5-[7-({5-[5-fluoro-3-(trifluoromethyl)phenyl](1,2,4-oxadiazol-3-yl)}methoxy)-4-oxochromen-3-yl]-1,3-dihydropyrimidine-2,4-dione.

### EXAMPLE 4

### Preparation of a Compound of Formula I

A. Preparation of a Compound of Formula I in which R¹ is 2-[5-fluoro-3-(trifluoromethyl)phenyl]-1,3-oxazole, R² is 4-Hydroxy, R³ is Hydrogen, X Y and Z are —CH—, V is Oxygen, and W is Methylene

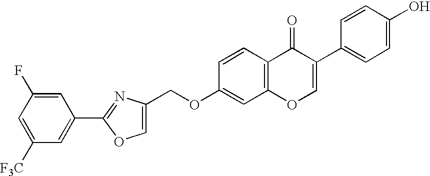

4′,7-Dihydroxyisoflavone (101.7 mg, 0.40 mmol), 4-(chloromethyl)-2-[5-fluoro-3-(trifluoromethyl)phenyl]-1,3-oxazole, prepared as described in Example 1 (111.8 mg, 040 mmol, 1.0 equiv.), sodium iodide (59.6 mg, 0.40 mmol, 1.0 equiv), and potassium hydroxide powder (22.4 mg, 0.4 mmol, 1.0 equiv) were placed in a 25 mL flask equipped with a condenser. To the flask was added dimethylsulfoxide (3 mL) at room temperature under nitrogen. The solution was heated at 60° C. for 1 hour. To the mixture were added water (30 mL) and the whole was extracted with ethyl acetate (30 mL×3). The combined organic layers were washed with brine (30 mL) and dried with Na₂SO₄, to give a crude mixture as colorless oil (204.7 mg). The crude mixture was purified by column-chromatography (silica gel=25 g, eluting with hexane/ethyl acetate=7:1) to give crude product (149.3 mg) as colorless crystals. Recrystallization of this crude product gave 7-({2-[5-fluoro-3-(trifluoromethyl)phenyl]-(1,3-oxazol-4-yl)}methoxy)-3-(4-hydroxyphenyl)chromen-4-one as a colorless powder.

### B.

Similarly, following the procedure of Example 4A above, substituting other compounds of formula (4) for 4-(chloromethyl)-2-[5-fluoro-3-(trifluoromethyl)phenyl]-1,3-oxazole, the following compounds of Formula I were prepared:
3-(4-hydroxyphenyl)-7-({2-[3-(trifluoromethyl)phenyl](1,3-oxazol-4-yl)}methoxy)chromen-4-one;
7-({2-[5-fluoro-3-(trifluoromethyl)phenyl](1,3-oxazol-4-yl)}methoxy)-3-(4-hydroxyphenyl)chromen-4-one;
3-(4-hydroxyphenyl)-7-{[2-(3,4,5-trifluorophenyl)(1,3-oxazol-4-yl)]methoxy}chromen-4-one;
7-{[2-(3,5-difluorophenyl)(1,3-oxazol-4-yl)]methoxy}-3-(4-hydroxyphenyl)chromen-4-one;
7-{[2-(3,4-difluorophenyl)(1,3-oxazol-4-yl)]methoxy}-3-(4-hydroxyphenyl)chromen-4-one;
7-{[2-(4-fluorophenyl)(1,3-oxazol-4-yl)]methoxy}-3-(4-hydroxyphenyl)chromen-4-one; and
7-{[2-(4-chlorophenyl)(1,3-oxazol-4-yl)]methoxy}-3-(4-hydroxyphenyl)chromen-4-one.

### EXAMPLE 5

### Preparation of a Compound of Formula I

A. Preparation of a Compound of Formula I in which R¹ is 3-(Trifluoromethyl)phenyl[1,2,4]oxadiazolyl R² is 4-Hydroxy, R³ is Hydrogen, X Y and Z are —CH—, V is Oxygen, and W is Methylene

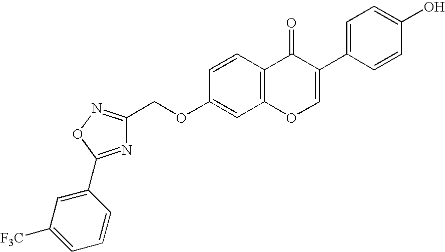

A mixture of daidzein (100 mg, 0.4 mmol), 3-chloromethyl-5-(3-trifluoromethyl(phenyl[1,2,4]oxadiazole (108 mg, 0.41 mmol) and potassium carbonate (0.63 mg, 0.45 mmol) in anhydrous N,N-dimethylformamide (2 ml) was heated with stirring under argon at 80° C. for 4.5 hours. After cooling to room temperature, the mixture was quenched with about 12 ml of water, and stirred for 30 minutes. The precipitate formed was filtered off, washed three times with water, and dried under vacuum to provide crude product (152 mg). Chromatography of the crude product on silica gel, eluting with 5% to 50% ethyl acetate/hexanes, provided pure 3-(4-hydroxyphenyl)-7-({5-[3-(trifluoromethyl)phenyl](1,2,4-oxadiazol-3-yl)}methoxy)chromen-4-one.

¹H NMR (400 MHz, DMSO-d₆) δ: 9.58 (s, 1H), 8.48-8.39 (m, 3H), 8.12 (d, 1H, J=8.0 Hz), 8.08 (d, 1H, J=8.8 Hz), 7.92 (t, 1H, J=8.8 Hz), 7.42-7.38 (m, 3H), 7.23 (d, 1H, J=9.2 Hz), 6.82 (d, 2H, J=8.8 Hz), 5.61 (s, 2H). LC/MS analysis: t_{R}=21.98 min (linear gradient B 5%→90%), (ESI) m/z 481.5 (M+H)⁺.

B. Alternative Preparation of a Compound of Formula I in which R¹ is 3-(Trifluoromethyl)phenyl[1,2,4]oxadiazolyl R² is 4-Hydroxy R³ is Hydrogen, X, Y and X are —CH—, V is Oxygen, and W is Methylene

To a suspension of daidzein (2.0 g, 7.87 mmol) in acetone (80 ml) 2 N aqueous potassium hydroxide (3.94 ml, 7.87 mmol) was added, and the mixture was sonicated for a few minutes. To this mixture was added 3-chloromethyl-5-(3-trifluoromethylphenyl)-[1,2,4]oxadiazole (2.17 g, 8.26 mmol), and the reaction mixture was refluxed for 3 days. The mixture was concentrated under reduced pressure, and the residue dissolved in methanol, mixed with silica gel, and the solvent removed under reduced pressure. Purification by flash column chromatography, eluting with methylene chloride/methanol (95/5 to 90/10) provided pure 3-(4-hydroxyphenyl)-7-({5-[3-(trifluoromethyl)phenyl]-(1,2,4-oxadiazol-3-yl)}methoxy)chromen-4-one as a white solid.

C. Preparation of Compounds of Formula I in which R³ is Hydrogen, X Y and Z are —CH— and V is Oxygen, Varying R¹ and R²

Similarly, following the procedures of Example 5A or 5B above, replacing 3-chloromethyl-5-(3-trifluoromethylphenyl)-[1,2,4]oxadiazole by other compounds of formula R¹CH₂X, where R¹ and X are as defined above, the following compounds of Formula I were prepared.
3-{[3-(4-hydroxyphenyl)-4-oxochromen-7-yloxy]methyl}benzoic acid; ¹H NMR (400 MHz, DMSO-d₆) δ: 13.1 (br s, 1H), 9.59 (br s, 1H), 8.38 (s, 1H), 8.08 (s, 1H), 8.05 (d, 1H, J=9.0 Hz), 7.94 (d, 1H, J=7.8 Hz), 7.75 (d, 1H, J=7.7 Hz), 7.56 (dd, 1H, J=7.5 Hz, J=7.8 Hz), 7.40 (d, 2H, J=8.7 Hz), 7.29 (d, 1H, J=1.9 Hz), 7.18 (dd, 1H, J=1.9 Hz, J=9.0 Hz), 6.82 (d, 2H, J=8.7 Hz), 5.37 (s, 2H). (ESI) m/z 389 (M+H).
3-(4-hydroxyphenyl)-7-[(5-phenyl(1,2,4-oxadiazol-3-yl))methoxy]chromen-4-one; ¹H NMR (300 MHz, DMSO-d₆) δ: 9.58 (s, 1H), 8.41 (s, 1H), 8.15 (d, 2H, J=7.2 Hz), 8.08 (d, 1H, J=9.0 Hz), 7.72-7.63 (m, 3H), 7.42-7.38 (m, 3H), 7.23 (d, 1H, J=9.0 Hz), 6.82 (d, 2H, J=8.7 Hz), 5.58 (s, 2H). (ESI) m/z 413.4 (M+H)⁺.
3-{[3-(4-hydroxyphenyl)-4-oxochromen-7-yloxy]methyl}benzenecarbonitrile; (ESI) m/z 370 (M+H)+.
3-{[3-(4-hydroxyphenyl)-4-oxochromen-7-yloxy]methyl}benzamide; ¹H NMR (300 MHz, DMSO-d₆) δ: 9.56 (s, 1H), 8.41 (s, 1H), 8.35 (d, 2H, J=8.1 Hz), 8.09-8.01 (m, 3H), 7.40 (m, 3H), 7.22 (dd, 1H, J=8.8, 2.1 Hz), 6.82 (d, 2H, J=8.7 Hz), 5.61 (s, 2H). (ESI) m/z 481.6 (M+H)⁺
3-(4-hydroxyphenyl)-7-{[5-(2-methoxyphenyl)(1,2,4-oxadiazol-3-yl)]methoxy}chromen-4-one; ¹H NMR (400 MHz, DMSO-d₆) δ: 9.57 (s, 1H), 8.40 (s, 1H), 8.07 (d, 1H, J=8.8 Hz), 8.03 (dd, 1H, J=8.0, 1.6 Hz), 7.69 (m, 1H), 7.42-7.15 (m, 6H), 6.82 (d, 2H, J=8.4 Hz), 5.56 (s, 2H), 3.95 (s, 3H). (ESI) m/z 443.3 (M+H)⁺
3-(4-hydroxyphenyl)-7-{[3-(trifluoromethyl)phenyl]methoxy}chromen-4-one; (K-28-AR-1) ¹H NMR (400 MHz, DMSO-d₆) d: 9.55 (s, 1H), 8.39 (s, 1H), 8.06 (d, 1H, J=8.8 Hz), 7.89 (s, 1H), 7.84-7.66 (m, 3H), 7.41 (d, 2H, 8.4 Hz), 7.29 (s, 1H), 7.20 (d, 1H, J=8.4 Hz), 6.82 (d, 2H, J=8.4 Hz), 5.40 (s, 2H). (ESI) m/z 413 (M+H)⁺.
3-(4-hydroxyphenyl)-7-{[4-methoxy-3-(trifluoromethyl)phenyl]methoxy}chromen-4-one; (DM-K-4-P3); ¹H NMR (300 MHz, DMSO-d₆) δ: 9.54 (s, 1H), 8.43-8.40 (m, 2H), 8.26 (d, 1H, J=1.8 Hz), 8.07 (d, 1H, J=8.9 Hz), 7.54 (d, 1H, J=8.9 Hz), 7.41 (d, 2H, J=8.7 Hz), 7.37 (d, 1H, J=2.4 Hz), 7.21 (dd, 1H, J=2.4 Hz, J=8.9 Hz), 6.82 (d, 2H, J=8.7 Hz), 5.56 (s, 2H), 4.03 (s, 3H). (ESI) m/z 511 (M+H)⁺
7-{[3-fluoro-5-(trifluoromethyl)phenyl]methoxy}-3-(4-hydroxyphenyl)chromen-4-one; (DM-K-28-AR-2), (ESI) m/z 431 (M+H)⁺.
7-({5-[3-fluoro-5-(trifluoromethyl)phenyl](1,2,4-oxadiazol-3-yl)}methoxy)-3-(4-hydroxyphenyl)chromen-4-one; ¹H NMR (400 MHz, DMSO-d₆) δ: 9.57 (s, 1H), 8.42 (s, 1H), 8.33 (d, 1H, J=8.4 Hz), 8.26 (s, 1H), 8.17 (d, 1H, J=8.4 Hz), 8.08 (d, 1H, J=8.8 Hz), 7.41 (m, 3H), 7.22 (dd, 1H, J=9.2, 2.0 Hz), 6.82 (d, 2H, J=8.8 Hz), 5.62 (s, 2H), (ESI) m/z 499 (M+H)⁺
7-({5-[4-fluoro-3-(trifluoromethyl)phenyl](1,2,4-oxadiazol-3-yl)}methoxy)-3-(4-hydroxyphenyl)chromen-4-one; ¹H NMR (300 MHz, DMSO-d₆) δ: 9.54 (s, 1H), 8.55-8.48 (m, 1H), 8.44-8.40 (m, 2H), 8.07 (d, 1H, J=8.9 Hz), 7.83 (dd, 1H, J=9.8 Hz, J=9.5 Hz), 7.41 (d, 2H, J=8.6 Hz), 7.38 (d, 1H, J=2.4 Hz), 7.21 (dd, 1H, J=2.4 Hz, J=8.9 Hz), 6.82 (d, 2H, J=8.6 Hz), 5.59 (s, 2H), (ESI) m/z 499 (M+H)⁺.
7-({5-[2,5-bis(trifluoromethyl)phenyl](1,2,4-oxadiazol-3-yl)}methoxy)-3-(4-hydroxyphenyl)chromen-4-one; ¹H NMR (400 MHz, DMSO-d₆) δ: 9.57 (s, 1H), 8.52 (s, 1H), 8.42 (s, 1H), 8.38-8.31 (m, 2H), 8.08 (d, 1H, J=9.0 Hz), 7.41 (d, 2H, 8.7 Hz), 7.40 (s, 1H), 7.22 (dd, 1H, J=1.9 Hz, J=9.0 Hz), 6.82 (d, 2H, J=8.7 Hz), 5.66 (s, 2H), (ESI) m/z 549 (M+H)⁺.
prop-2-enyl 3-(3-{[3-(4-hydroxyphenyl)-4-oxochromen-7-yloxy]methyl}-1,2,4-oxadiazol-5-yl)benzoate; (ESI) m/z 497 (M+H)⁺.
prop-2-enyl 3-{[3-(4-hydroxyphenyl)-4-oxochromen-7-yloxy]methyl}benzoate; LC/MS analysis: t_{R}=23.62 min (isocratic, 65% B), (ESI) m/z 429 (M+H)⁺.
methyl 3-{[3-(4-hydroxyphenyl)-4-oxochromen-7-yloxy]methyl}benzoate; ¹H NMR (400 MHz, DMSO-d₆) δ: 9.54 (s, 1H), 8.38 (s, 1H), 8.10 (s, 1H), 8.05 (d, 1H, J=8.8 Hz), 7.96 (d, 1H, J=7.7 Hz), 7.79 (d, 1H, J=7.5 Hz), 7.60 (dd, 1H, J=7.5 Hz, J=7.7 Hz), 7.41 (d, 2H, J=8.5 Hz), 7.27 (s, 1H), 7.18 (dd, 1H, J=1.5 Hz, J=9.0 Hz), 6.82 (d, 2H, J=8.5 Hz), 5.38 (s, 2H), 3.88 (s, 3H), (ESI) m/z 403 (M+H)⁺.
ethyl 4-{[3-(4-hydroxyphenyl)-4-oxochromen-7-yloxy]methyl}benzoate;, (ESI) m/z 417 (M+H)⁺.
methylethyl 3-{[3-(4-hydroxyphenyl)-4-oxochromen-7-yloxy]methyl}benzoate; ¹H NMR (400 MHz, DMSO-d₆) d: 9.56 (s, 1H), 8.39 (s, 1H), 8.08 (s, 1H), 8.05 (d, 1H, J=9.0 Hz), 7.95 (d, 1H, J=7.8 Hz), 7.78 (d, 1H, J=7.7 Hz), 7.58 (dd, 1H, J=7.6 Hz, J=7.9 Hz), 7.41 (d, 2H, J=8.3 Hz), 7.28 (d, 1H, J=1.9 Hz), 7.18 (dd, 1H, J=1.9 Hz, J=9.0 Hz), 6.82 (d, 2H, J=8.3 Hz), 5.37 (s, 2H), 5.18-5.14 (m, 1H), 1.33 (d, 6H, J=6.3 Hz), (ESI) m/z 431 (M+H)⁺.
methyl 4-{[3-(4-hydroxyphenyl)-4-oxochromen-7-yloxy]methyl}benzoate.
4-{[3-(4-hydroxyphenyl)-4-oxochromen-7-yloxy]methyl}benzoic acid; (ESI) m/z 389 (M+H)⁺.
4-{[3-(4-hydroxyphenyl)-4-oxochromen-7-yloxy]methyl}benzamide; ¹H NMR (400 MHz, DMSO-d₆) δ: 9.54 (s, 1H), 8.38 (s, 1H), 8.07-8.04 (m, 3H), 7.87 (d, 1H, J=8.0 Hz), 7.66 (d, 1H, J=7.6 Hz), 7.51 (m, 1H), 7.41 (m, 3H), 7.28 (d, 1H, J=2.0 Hz), 7.18 (dd, 1H, J=9.2, 2.0 Hz), 6.82 (d, 2H, J=8.4 Hz), 5.33 (s, 2H), (ESI) m/z 388/389.
3-(4-hydroxyphenyl)-7-({5-[4-(trifluoromethyl)phenyl](1,2,4-oxadiazol-3-yl)}methoxy)chromen-4-one; ¹H NMR (300 MHz, DMSO-d₆) d: 9.56 (s, 1H), 8.41 (s, 1H), 8.35 (d, 2H, J=8.1 Hz), 8.09-8.01 (m, 3H), 7.40 (m, 3H), 7.22 (dd, 1H, J=8.8, 2.1 Hz), 6.82 (d, 2H, J=8.7 Hz), 5.61 (s, 2H), (ESI) m/z 481.6 (M+H)⁺.
3-(4-hydroxyphenyl)-7-{[5-(3-methoxyphenyl)(1,2,4-oxadiazol-3-yl)]methoxy}chromen-4-one;
7-({5-[3,5-bis(trifluoromethyl)phenyl] (1,2,4-oxadiazol-3-yl)}methoxy)-3-(4-hydroxyphenyl)chromen-4-one; ¹H NMR (400 MHz, DMSO-d₆) δ: 9.57 (d, 1H, J=1.6 Hz), 8.69 (s, 2H), 8.56 (s, 1H), 8.41 (d, 1H, J=2.0 Hz), 8.07 (dd, 1H, J=8.8, 2.0 Hz), 7.40 (m, 3H), 7.22 (d, 1H, J=8.8 Hz), 6.82 (d, 2H, J=6.4 Hz), 5.63 (s, 2H), (ESI) m/z 549.1 (M+H)⁺
3-(3-{[3-(4-hydroxyphenyl)-4-oxochromen-7-yloxy]methyl}-1,2,4-oxadiazol-5-yl)benzenecarbonitrile;, (ESI) m/z 438 (M+H)⁺
3-(3-{[3-(4-hydroxyphenyl)-4-oxochromen-7-yloxy]methyl}-1,2,4-oxadiazol-5-yl)benzoic acid;
7-{[5-(3-fluorophenyl)(1,2,4-oxadiazol-3-yl)]methoxy}-3-(4-hydroxyphenyl)chromen-4-one. ¹H NMR (300 MHz, DMSO-d₆) δ: 9.55 (s, 1H), 8.40 (s, 1H), 8.08 (d, 1H, J=8.7 Hz), 8.00 (d, 1H, J=7.8 Hz), 7.94 (d, 1H, J=9.0 Hz), 7.73-7.60 (m, 2H), 7.42-7.38 (m, 3H), 7.21 (dd, 1H, J=9.0, 2.4 Hz), 6.82 (d, 2H, J=8.7 Hz), 5.59 (s, 2H), (ESI) m/z 431 (M+H)⁺.
3-(4-hydroxyphenyl)-7-[(3-phenyl(1,2,4-oxadiazol-5-yl))methoxy]chromen-4-one; (ESI) m/z 413.4 (M+H)⁺.
3-(4-hydroxyphenyl)-7-({3-[3-(trifluoromethyl)phenyl](1,2,4-oxadiazol-5-yl)}methoxy)chromen-4-one; (ESI) m/z 481.6 (M+H)⁺.
3-(4-hydroxyphenyl)-7-({3-[4-chlorophenyl](1,2,4-oxadiazol-5-yl)}methoxy)chromen-4-one; (ESI) m/z 447.2 (M+H)⁺.
3-(4-hydroxyphenyl)-2-(trifluoromethyl)-7-({5-[3-(trifluoromethyl)phenyl](1,2,4-oxadiazol-3-yl)}methoxy)chromen-4-one; ¹H NMR (300 MHz, DMSO-d₆) δ: 9.64 (s, 1H), 8.45 (d, 1H, J=7.8 Hz), 8.39 (s, 1H), 8.17-7.83 (m, 3H), 7.53 (d, 1H, J=2.4 Hz), 7.27 (dd, 1H, J=8.7, 2.1 Hz), 7.08 (d, 2H, J=8.7 Hz), 6.82 (d, 2H, J=8.4 Hz), 5.65 (s, 2H), (ESI) m/z 549 (M+H)⁺.
7-({5-[5-fluoro-3-(trifluoromethyl)phenyl](1,2,4-oxadiazol-3-yl)}methoxy)-3-(4-hydroxyphenyl)-2-(trifluoromethyl)chromen-4-one; ¹H NMR (400 MHz, DMSO-d₆) δ: 9.67 (s, 1H), 8.32 (d, 1H, J=8.4 Hz), 8.25 (s, 1H), 8.17 (d, 1H, J=8.4 Hz), 8.02 (d, 1H, J=8.4 Hz), 7.54 (d, 1H, J=1.6 Hz), 7.27 (dd, 1H, J=8.8, 2.4 Hz), 7.08 (d, 2H, J=8.0 Hz), 6.82 (d, 2H, J=8.8 Hz), 5.66 (s, 2H). (ESI) m/z 567 (M+H)⁺
3-(4-hydroxyphenyl)-7-({5-[4-methoxy-3-(trifluoromethyl)phenyl](1,2,4-oxadiazol-3-yl)}methoxy)-2-(trifluoromethyl)chromen-4-one; (ESI) m/z 579 (M+H)⁺
3-{[3-(4-hydroxyphenyl)-4-oxochromen-7-yloxy]methyl}benzenecarbonitrile;
3-(3-{[3-(4-hydroxyphenyl)-4-oxochromen-7-yloxy]methyl}-1,2,4-oxadiazol-5-yl)benzoic acid.
3-(4-hydroxyphenyl)-7-({5-[3-(trifluoromethyl)phenyl]isoxazol-3-yl}methoxy)chromen-4-one;
7-{[5-(trifluoromethyl)(3-pyridyl)]methoxy}-3-(4-{[6-(trifluoromethyl)(3-pyridyl)]methoxy}phenyl)chromen-4-one;
3-(4-hydroxyphenyl)-7-[(5-(3-pyridyl)(1,2,4-oxadiazol-3-yl))methoxy]chromen-4-one;
3-(4-hydroxyphenyl)-7-[(5-(2-pyridyl)(1,2,4-oxadiazol-3-yl))methoxy]chromen-4-one;
methyl 2-{[3-(4-hydroxyphenyl)-4-oxochromen-7-yloxy]methyl}-1,3-oxazole-5-carboxylate;
7-{[5-(4-fluorophenyl)(1,2,4-oxadiazol-3-yl)]methoxy}-3-{4-[(methylsulfonyl)amino]-phenyl}chromen-4-one;
2-{[3-(4-hydroxyphenyl)-4-oxochromen-7-yloxy]methyl}-1,3-oxazole-5-carboxylic acid;
methyl 3-({3-[4-((1Z)-1-amino-2-methoxy-2-azavinyl)phenyl]-4-oxochromen-7-yloxy}methyl)benzoate;
7-{2-[4-(4-chlorophenyl)pyrazolyl]ethoxy}-3-(4-hydroxyphenyl)chromen-4-one;
3-(4-hydroxyphenyl)-7-[(6-pyrazolyl(3-pyridyl))methoxy]chromen-4-one;
7-[(2R)-2-hydroxy-3-({[3-(trifluoromethyl)phenyl]methyl}amino)propoxy]-3-(4-hydroxyphenyl)chromen-4-one;
3-(4-hydroxyphenyl)-7-[({[3-(trifluoromethyl)phenyl]methyl}amino)methoxy]chromen-4-one;
7-((2R)-3-{[(3,5-difluorophenyl)methyl]amino}-2-hydroxypropoxy)-3-(4-hydroxyphenyl)chromen-4-one;
7-(3-{[(1R)-1-(4-fluorophenyl)ethyl]amino}-2-oxopropoxy)-3-(4-hydroxyphenyl)chromen-4-one;
3-(4-hydroxyphenyl)-7-(3-phenylpropoxy)chromen-4-one;
7-{[5-(3-fluorophenyl)(1,3,4-oxadiazol-2-yl)]methoxy}-3-(4-hydroxyphenyl)chromen-4-one;
3-(4-hydroxyphenyl)-7-{[3-(trifluoromethyl)phenyl]ethoxy}chromen-4-one;
3-(4-hydroxyphenyl)-7-({5-[3-(trifluoromethyl)phenyl] (1,3,4-oxadiazol-2-yl)}methoxy)chromen-4-one;
3-(4-hydroxyphenyl)-7-[(2-phenyl(1,3-oxazol-5-yl))methoxy]chromen-4-one;
7-({5-[3,5-bis(trifluoromethyl)phenyl]isoxazol-3-yl}methoxy)-3-(4-hydroxyphenyl)chromen-4-one;
3-(4-hydroxyphenyl)-7-({5-[3-(trifluoromethyl)phenyl]isoxazol-3-yl}methoxy)chromen-4-one;
3-{4-[(methylsulfonyl)amino]phenyl}-7-[(2-phenyl(1,3-oxazol-4-yl))methoxy]chromen-4-one;
2-[3-(4-hydroxyphenyl)-4-oxochromen-7-yloxy]-N-[3-(trifluoromethyl)phenyl]-acetamide;
7-{[5-(2-chlorophenyl)(1,3,4-thiadiazol-2-yl)]methoxy}-3-(4-hydroxyphenyl)chromen-4-one;
4-[7-({4-methyl-2-[4-(trifluoromethyl)phenyl] (1,3-thiazol-5-yl)}methoxy)-4-oxochromen-3-yl]benzenecarbonitrile;
3-{4-[(methylsulfonyl)amino]phenyl}-7-({4-methyl-2-[4-(trifluoromethyl)phenyl](1,3-thiazol-5-yl)}methoxy)chromen-4-one;
3-(6-methoxy(3-pyridyl))-7-({4-methyl-2-[4-(trifluoromethyl)phenyl] (1,3-thiazol-5-yl)}methoxy)chromen-4-one;
4-[7-({5-[5-fluoro-3-(trifluoromethyl)phenyl](1,3,4-oxadiazol-2-yl)}methoxy)-4-oxochromen-3-yl]benzenecarbonitrile;
4-[4-oxo-7-({3-[3-(trifluoromethyl)phenyl]isoxazol-5-yl}methoxy)chromen-3-yl]benzenecarbonitrile;
7-({5-[3-fluoro-5-(trifluoromethyl)phenyl](1,2,4-oxadiazol-3-yl)}methoxy)-3-{4-[(methylsulfonyl)amino]phenyl}chromen-4-one;
7-({5-[3-fluoro-5-(trifluoromethyl)phenyl](1,2,4-oxadiazol-3-yl)}methoxy)-3-[4-(methylsulfonyl)phenyl]chromen-4-one;
4-[7-({5-[3-fluoro-5-(trifluoromethyl)phenyl](1,2,4-oxadiazol-3-yl)}methoxy)-4-oxochromen-3-yl]benzamide;
3-(3-acetylphenyl)-7-({5-[3-fluoro-5-(trifluoromethyl)phenyl](1,2,4-oxadiazol-3-yl)}methoxy)chromen-4-one;
7-({5-[3-fluoro-5-(trifluoromethyl)phenyl](1,3,4-oxadiazol-2-yl)}methoxy)-3-(4-hydroxyphenyl)chromen-4-one;
7-({5-[3-fluoro-5-(trifluoromethyl)phenyl](1,2,4-oxadiazol-3-yl)}methoxy)-3-(5-hydropyrazol-4-yl)chromen-4-one;
ethyl 3-[7-({3-[3-fluoro-5-(trifluoromethyl)phenyl](1,2,4-oxadiazol-5-yl)}ethoxy)-4-oxochromen-3-yl]benzoate;
3-(4-hydroxyphenyl)-7-({2-[4-(trifluoromethyl)phenyl](1,3-thiazol-5-yl)}methoxy)chromen-4-one;
7-[2-(3-fluorophenyl)-2-oxoethoxy]-3-(4-hydroxyphenyl)chromen-4-one;
7-({5-[3-fluoro-5-(trifluoromethyl)phenyl](1,2,4-oxadiazol-3-yl)}ethoxy)-3-(4-hydroxyphenyl)chromen-4-one;
7-({5-[3-fluoro-5-(trifluoromethyl)phenyl](1,2,4-oxadiazol-3-yl)}methoxy)-3-(4-{[(4-methylphenyl)sulfonyl]amino}phenyl)chromen-4-one;
7-{[5-(2-chlorophenyl)(1,3,4-oxadiazol-2-yl)]methoxy}-3-(4-hydroxyphenyl)chromen-4-one;
7-{[5-(4-fluorophenyl)(1,3,4-oxadiazol-2-yl)]methoxy}-3-(4-hydroxyphenyl)chromen-4-one;
3-(4-hydroxyphenyl)-7-(4-pyridylmethoxy)chromen-4-one;
3-{4-[(methylsulfonyl)amino]phenyl}-7-({2-[4-(trifluoromethyl)phenyl](1,3-thiazol-5-yl)}methoxy)chromen-4-one;
2-[3-(4-hydroxyphenyl)-4-oxochromen-7-yloxy]-N-[2-(trifluoromethyl)phenyl]-acetamide;
3-(4-hydroxyphenyl)-7-{2-oxo-2-[2-(trifluoromethyl)phenyl]ethoxy}chromen-4-one;
3-(1H-indazol-5-yl)-7-({5-[5-fluoro-3-(trifluoromethyl)phenyl] (1,2,4-oxadiazol-3-yl)}methoxy)chromen-4-one;
3-(4-hydroxyphenyl)-7-(2-phenylethoxy)chromen-4-one;
2-[3-(4-hydroxyphenyl)-4-oxochromen-7-yloxy]ethanenitrile;
7-[2-(4-chlorophenoxy)ethoxy]-3-(4-hydroxyphenyl)chromen-4-one;
5-{4-[7-({5-[5-fluoro-3-(trifluoromethyl)phenyl](1,2,4-oxadiazol-3-yl)}methoxy)-4-oxochromen-3-yl]phenyl}-1,3,5,6-tetrahydropyrimidine-2,4-dione;
N-[(1R)-1-(4-fluorophenyl)ethyl]-2-[3-(4-hydroxyphenyl)-4-oxochromen-7-yloxy]acetamide;
3-(4-hydroxyphenyl)-7-(2-pyridylmethoxy)chromen-4-one;
2-fluoro-5-[7-({5-[5-fluoro-3-(trifluoromethyl)phenyl](1,2,4-oxadiazol-3-yl)}methoxy)-4-oxochromen-3-yl]benzenecarbonitrile;
7-(2-pyridylmethoxy)-3-[4-(2-pyridylmethoxy)phenyl]chromen-4-one;
3-(4-hydroxyphenyl)-7-[(5-(4-pyridyl)(1,2,4-oxadiazol-3-yl))ethoxy]chromen-4-one;
3-(4-hydroxyphenyl)-7-[(5-(3-pyridyl)(1,2,4-oxadiazol-3-yl))ethoxy]chromen-4-one;
3-(4-hydroxyphenyl)-7-[(5-(2-pyridyl)(1,2,4-oxadiazol-3-yl))ethoxy]chromen-4-one;
3-(4-hydroxyphenyl)-7-{[5-(trifluoromethyl)(3-pyridyl)]methoxy}chromen-4-one;
7-{[5-(4-chlorophenyl)isoxazol-3-yl]methoxy}-3-(4-hydroxyphenyl)chromen-4-one;
7-{[5-(3,4-dichlorophenyl)isoxazol-3-yl]methoxy}-3-(4-hydroxyphenyl)chromen-4-one;
7-{[5-(4-chlorophenyl)isoxazol-3-yl]methoxy}-3-(4-hydroxyphenyl)chromen-4-one;
7-[(2R)-2-hydroxy-3-({[3-(trifluoromethyl)phenyl]methyl}amino)propoxy]-3-(4-hydroxyphenyl)chromen-4-one;
3-(4-hydroxyphenyl)-7-[2-({[3-(trifluoromethyl)phenyl]methyl}amino)ethoxy]chromen-4-one;
7-((2R)-3-{[(3,5-difluorophenyl)methyl]amino}-2-hydroxypropoxy)-3-(4-hydroxyphenyl)chromen-4-one;
methyl 2-{[3-(4-hydroxyphenyl)-4-oxochromen-7-yloxy]methyl}-1,3-oxazole-4-carboxylate;
   which was hydrolyzed under standard hydrolysis conditions to give:
2-{[3-(4-hydroxyphenyl)-4-oxochromen-7-yloxy]methyl}-1,3-oxazole-4-carboxylic acid;
N-[(1S)-1-(4-fluorophenyl)ethyl]-2-[3-(4-hydroxyphenyl)-4-oxochromen-7-yloxy]acetamide;
7-{[5-(4-fluorophenyl)(1,2,4-oxadiazol-3-yl)]methoxy}-3-(4-hydroxyphenyl)chromen-4-one;
7-{[5-(4-fluorophenyl)(1,2,4-oxadiazol-3-yl)]methoxy}-3-{4-[(methylsulfonyl)amino]phenyl}chromen-4-one;
7-{3-[4-(4-chlorophenyl)pyrazolyl]propoxy}-3-(4-hydroxyphenyl)chromen-4-one;
3-(4-hydroxyphenyl)-7-(3-phenylpropoxy)chromen-4-one;
3-(4-hydroxyphenyl)-7-[(6-pyrazolyl(3-pyridyl))methoxy]chromen-4-one;
7-((2R)-2-hydroxy-3-phenylpropoxy)-3-(4-hydroxyphenyl)chromen-4-one;
3-(4-hydroxyphenyl)-7-[(5-(3-pyridyl)(1,3,4-oxadiazol-2-yl))methoxy]chromen-4-one;
3-[(2-hydroxy-3-{4-[(methylsulfonyl)amino]phenyl}-4-oxochromen-7-yloxy)methyl]-benzoic acid;
7-{[5-(4-fluorophenyl)(1,3,4-oxadiazol-2-yl)]ethoxy}-3-(4-hydroxyphenyl)chromen-4-one;
3-(4-hydroxyphenyl)-7-[(5-(3-pyridyl)(1,3,4-oxadiazol-2-yl))ethoxy]chromen-4-one;
3-(4-hydroxyphenyl)-7-[(3-(3-pyridyl)(1,2,4-oxadiazol-5-yl))methoxy]chromen-4-one;
3-(4-hydroxyphenyl)-7-({3-[3-(trifluoromethyl)phenyl](1,2,4-oxadiazol-5-yl)}methoxy)chromen-4-one;
3-(4-hydroxyphenyl)-7-[(5-(3-pyridyl)(1,3,4-oxadiazol-2-yl))ethoxy]chromen-4-one;
3-(4-hydroxyphenyl)-7-[(5-(4-pyridyl)(1,2,4-oxadiazol-3-yl))ethoxy]chromen-4-one;
(2-{[3-(4-hydroxyphenyl)-4-oxochromen-7-yloxy]methyl}(1,3-oxazol-4-yl))-N-methylcarboxamide;
4-{[3-(4-hydroxyphenyl)-4-oxochromen-7-yloxy]methyl}-7-methoxychromen-2-one;
7-{[5-(4-fluorophenyl)(1,3,4-oxadiazol-2-yl)]methoxy}-3-{4-[(methylsulfonyl)amino]-phenyl}chromen-4-one;
7-{[5-(3-aminophenyl)(1,3,4-oxadiazol-2-yl)]methoxy}-3-(4-hydroxyphenyl)chromen-4-one;
ethyl 1-{2-[3-(4-hydroxyphenyl)-4-oxochromen-7-yloxy]ethyl}pyrazole-4-carboxylate;
7-{2-[4-(3-chlorophenyl)piperazinyl]ethoxy}-3-(4-hydroxyphenyl)chromen-4-one;
3-(4-hydroxyphenyl)-7-(2-{4-[3-(trifluoromethyl)phenyl]piperazinyl}ethoxy)chromen-4-one;
3-(4-hydroxyphenyl)-7-[(5-(2-pyridyl)isoxazol-3-yl)methoxy]chromen-4-one;
7-({3-[3-fluoro-5-(trifluoromethyl)phenyl](1,2,4-oxadiazol-5-yl)}ethoxy)-3-(4-hydroxyphenyl)chromen-4-one;
7-[2-(4-fluorophenyl)ethoxy]-3-(4-hydroxyphenyl)chromen-4-one;
7-((1R)-1-{3-[3-fluoro-5-(trifluoromethyl)phenyl](1,2,4-oxadiazol-5-yl)}ethoxy)-3-(4-hydroxyphenyl)chromen-4-one;
7-((1S)-1-{3-[3-fluoro-5-(trifluoromethyl)phenyl] (1,2,4-oxadiazol-5-yl)}ethoxy)-3-(4-hydroxyphenyl)chromen-4-one;
3-(4-hydroxyphenyl)-7-{2-[3-(trifluoromethyl)pyrazolyl]ethoxy}chromen-4-one; and
7-(1-{3-[3-fluoro-5-(trifluoromethyl)phenyl] (1,2,4-oxadiazol-5-yl)}-isopropoxy)-3-(4-hydroxyphenyl)chromen-4-one.

### D. Preparation of a Compound of Formula (3)

Similarly, following the procedures of Example 5A or 5B above, replacing 3-hydroxy isoflavone by commercially available isoflavones in which the 3-phenyl group is substituted with a nitro group and/or replacing 3-chloromethyl-5-(3-trifluoromethylphenyl)-[1,2,4]oxadiazole by other compounds of formula R¹CH₂X, where R¹ and X are as defined above, the following compounds of formula (3) were prepared.
methyl 3-{[3-(4-nitrophenyl)-4-oxochromen-7-yloxy]methyl}benzoate; (ESI) m/z 432 (M+H)⁺
3-(4-nitrophenyl)-7-({5-[3-(trifluoromethyl)phenyl](1,2,4-oxadiazol-3-yl)}methoxy)chromen-4-one; (ESI) m/z 510.5 (M+H).
7-({5-[5-fluoro-3-(trifluoromethyl)phenyl](1,2,4-oxadiazol-3-yl)}methoxy)-3-(4-nitrophenyl)chromen-4-one; (ESI) m/z 528.1 (M+H)⁺.
prop-2-enyl 3-(3-{[3-(4-nitrophenyl)-4-oxochromen-7-yloxy]methyl}-1,2,4-oxadiazol-5-yl)benzoate; (ESI) m/z 458 (M+H)⁺.
3-{[3-(4-nitrophenyl)-4-oxochromen-7-yloxy]methyl}benzenecarbonitrile; (ESI) m/z 399 (M+H)⁺.
methyl 3-{[3-(4-nitrophenyl)-4-oxochromen-7-yloxy]methyl}benzoate; (ESI) m/z 432 (M+H)⁺.
7-(benzothiazol-2-ylmethoxy)-3-(4-hydroxyphenyl)chromen-4-one.
   E Preparation of Compounds of Formula I in which R² is 4-Hydroxy, R³ is Hydrogen, X Y and Z are —CH—, V is Oxygen, and W is Methylene, Varying R¹

Similarly, following the procedures of Example 5A or 5B above, replacing 3-hydroxy isoflavone by commercially available isoflavones in which the 3-phenyl group is substituted with a nitro group and/or replacing 3-chloromethyl-5-(3-trifluoromethylphenyl)-[1,2,4]oxadiazole by other compounds of formula R¹CH₂X, where R¹ and X are as defined above, other compounds of Formula I are prepared.

### EXAMPLE 6

### Preparation of a Compound of Formula I

A. Preparation of a Compound of Formula I in which R¹ is (3-(1H-1,2,3,4-Tetrazol-5-yl)phenyl) 1,2,4-oxadiazol-5-yl) R² is 4-Hydroxy, R³ is Hydrogen, X Y and Z are —CH—, V is Oxygen, and W is Methylene

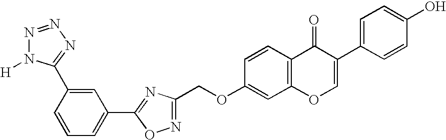

A mixture of 3-(3-{[3-(4-hydroxyphenyl)-4-oxochromen-7-yloxy]methyl}-1,2,4-oxadiazol-5-yl)benzenecarbonitrile (51 mg, 0.117 mmol), dibutyltin(IV) oxide (15 mg, 0.059 mmol, 0.5 equiv), and azidotrimethylsilane (81 mg, 0.702 mmol, 6 equiv) was microwaved at 150° C. for 20 minutes in 1,2-dimethoxyethane (0.6 ml). The reaction mixture was then dry-loaded onto a pre-packed column using silica gel and purified (silica gel, gradient, 100% CH₂Cl₂ to CH₂Cl₂/MeOH, 3:1) by flash chromatography to obtain the desired product protected by trimethylsilyl. This intermediate was suspended in acetonitrile (2 ml) and water (1 ml) and one drop of trifluoroacetic acid added. The volatile solvents were removed under vacuum to afford 3-(4-hydroxyphenyl)-7-{[5-(3-(1,2,3,4-tetraazol-5-yl)phenyl)(1,2,4-oxadiazol-3-yl)]methoxy}chromen-4-one (4 mg).

¹H NMR (400 MHz, DMSO-d₆) d: 9.57 (s, 1H), 8.82 (s, 1H), 8.42-8.33 (m, 3H), 8.09 (d, 1H, J=8.8 Hz), 7.92 (m, 1H), 7.41 (m, 3H), 7.24 (dd, 1H, J=8.8, 1.6 Hz), 6.82 (d, 2H, J=8.4 Hz), 5.62 (s, 2H). (ES−) m/z 479.2 (M−1)

B. Preparation of a Compound of Formula I in which R¹ is (3-(1H-1,2,3,4-Tetraazol-5-yl)phenyl) R² is 4-Hydroxy R³ is Hydrogen, X Y and X are —CH—, V is Oxygen, and W is Methylene

Similarly, starting with 3-{[3-(4-hydroxyphenyl)-4-oxochromen-7-yloxy]methyl}benzenecarbonitrile and following the procedure of 6A above, 3-(4-hydroxyphenyl)-7-[(3-(1H-1,2,3,4-tetrazol-5-yl)phenyl)methoxy]chromen-4-one was prepared.

¹H NMR (400 MHz, DMSO-d₆) d: 9.56 (s, 1H), 8.39 (s, 1H), 8.21 (s, 1H), 8.06 (m, 2H), 7.73-7.67 (m, 2H), 7.40 (d, 2H, J=8.4 Hz), 7.31-6.81 (m, 5H), 5.42 (s, 2H). (ESI) m/z 435 (M+Na)⁺, (ES−) m/z 411.1 (M−1)

C. Preparation of a Compound of Formula I in which R¹ is (3-(1H-1,2,3,4-Tetrazol-5-yl)phenyl)

Similarly, starting with other compounds of Formula I in which R¹ is phenyl substituted by cyano, and following the procedure of 6A above, other compounds of Formula I in which R¹ is 3-(1H-1,2,3,4-tetrazol-5-yl)phenyl are prepared.

### EXAMPLE 7

### Preparation of a Compound of Formula I

A. Preparation of a Compound of Formula I in which R¹ is Prop-2-enyl 3-benzoate and R² is Amino

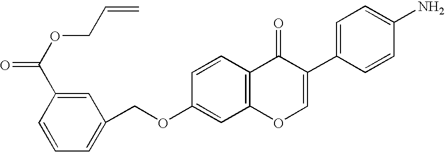

A suspension of 3-[3-(4-nitrophenyl)-4-oxo-4H-chromen-7-yloxymethyl]benzoic acid allyl ester (164.6 mg, 0.36 mmol) and sodium dithionite (188 mg, 1.08 mmol) in tetrahydrofuran (8 ml) and water (4 ml) was heated at 60-65° C. for 1 hour. Additional sodium dithionite (1.13 g, 6.48 mmol) was added in 5 portions over 2 hours. The reaction mixture was stirred at 60-65° C. overnight. ¹H NMR of the reaction mixture showed that the product was obtained without starting material. The reaction mixture was mixed with silica gel (2 g), solvent removed under reduced pressure, and the mixture applied to a column. The silica gel mixture was purified by flash chromatography, eluting with methylene chloride/methanol (98/2) to give prop-2-enyl 3-{[3-(4-aminophenyl)-4-oxochromen-7-yloxy]methyl}benzoate as a yellow solid (99.6 mg, 65%);. (ESI) m/z 428 (M+H)⁺.

### B. Preparation of a Compound of Formula I, Varying R¹

Similarly, replacing 3-[3-(4-nitrophenyl)-4-oxo-4H-chromen-7-yloxymethyl]benzoic acid allyl ester with other compounds of formula (3), and following the procedure of 7A above, the following compounds of formula (4) were prepared:

3-(4-aminophenyl)-7-({5-[3-(trifluoromethyl)phenyl](1,2,4-oxadiazol-3-yl)}methoxy)chromen-4-one; ¹H NMR (400 MHz, DMSO-d₆) d: 8.46 (d, 1H, J=7.9 Hz) 8.39 (s, 1H), 8.35 (s, 1H), 8.13 (d, 1H, J=7.6 Hz), 8.07 (d, 1H, J=8.9 Hz), 7.92 (dd, 1H, J=7.9 Hz, J=7.9 Hz), 7.37 (d, 1H, J=1.8 Hz), 7.27 (d, 2H, J=8.3 Hz), 7.21 (dd, 1H, J=1.8 Hz, J=8.9 Hz), 6.61 (d, 2H, J=8.3 Hz), 5.60 (s, 2H), 5.23 (s, 2H); (ESI) m/z 480 (M+H)⁺.
methyl 3-{[3-(4-aminophenyl)-4-oxochromen-7-yloxy]methyl}benzoate; (ESI) m/z 402 (M+H)⁺
7-({5-[5-fluoro-3-(trifluoromethyl)phenyl](1,2,4-oxadiazol-3-yl)}methoxy)-3-(4-aminophenyl)chromen-4-one;) (ESI) m/z 498.2 (M+H)⁺.
3-{[3-(4-aminophenyl)-4-oxochromen-7-yloxy]methyl}benzenecarbonitrile; (ESI) m/z 369 (M+H)⁺.
3-{[3-(4-aminophenyl)-4-oxochromen-7-yloxy]methyl}benzamide; (ESI) m/z 387 (M+H)⁺.

### C. Preparation of a Compound of Formula I, Varying R¹

Similarly, replacing 3-[3-(4-nitrophenyl)-4-oxo-4H-chromen-7-yloxymethyl]benzoic acid allyl ester with other compounds of formula (3), and following the procedure of 3A above, other compounds of Formula I are prepared.

### EXAMPLE 8

### Preparation of a Compound of Formula I

A. Preparation of a Compound of Formula I in which R¹ is 3-(Prop-2-enyl)benzoate, R² is 4-[(Methylsulfonyl)amino, R³ is Hydrogen, X, Y and Z are —CH—, V is Oxygen, and W is Methylene

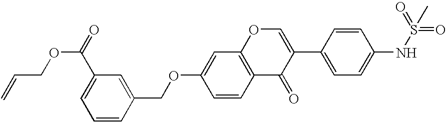

To a mixture of prop-2-enyl 3-{[3-(4-aminophenyl)-4-oxochromen-7-yloxy]methyl}benzoate (169.5 mg, 0.397 mmol) and anhydrous pyridine (34.5 mg, 0.44 mmol) in dry methylene chloride (3 ml) at 0° C. was added methanesulfonyl chloride (68.1 mg, 0.60 mmol). The mixture was then stirred at room temperature for 21 hours, then mixed with silica gel and the solvent removed under reduced pressure. Flash chromatography of the silica gel mixture, eluting with methylene chloride/methanol (99.5/0.5) gave prop-2-enyl 3-[(3-{4-[(methylsulfonyl)amino]phenyl}-4-oxochromen-7-yloxy)methyl]benzoate as a white solid (160.9 mg). (ESI) m/z 506 (M+H).⁺.

B. Preparation of Compounds of Formula I in which R² is 4-[(Methylsulfonyl)amino, R³ is Hydrogen, X Y and Z are —CH—, V is Oxygen, and W is Methylene, Varying R¹

Similarly, replacing prop-2-enyl 3-{[3-(4-aminophenyl)-4-oxochromen-7-yloxy]methyl}benzoate with other compounds of formula (4), and following the procedure of 8A above, the following compounds of Formula I in which R² is 4-[(methylsulfonyl)amino were prepared:

methyl 3-[(3-{4-[(methylsulfonyl)amino]phenyl}-4-oxochromen-7-yloxy)methyl]benzoate; ¹H NMR (400 MHz, DMSO-d₆) d: 9.84 (br s, 1H), 8.46 (s, 1H), 8.10 (s, 1H), 8.07 (d, 1H, J=8.9 Hz), 7.96 (d, 1H, J=7.8 Hz), 7.80 (d, 1H, J=7.7 Hz), 7.62-7.56 (m, 3H), 7.30 (s, 1H), 7.27 (d, 2H, J=8.1 Hz), 7.20 (dd, 1H, J=1.5 Hz, J=9.0 Hz), 5.39 (s, 2H), 3.03 (s, 3H). (ESI) m/z 480 (M+H)⁺.

3-{4-[(methylsulfonyl)amino]phenyl}-7-({5-[3-(trifluoromethyl)phenyl](1,2,4-oxadiazol-3-yl)}methoxy)chromen-4-one; ¹H NMR (300 MHz, DMSO-d₆) δ: 9.86 (s, 1H), 8.49 (s, 1H), 8.45 (d, 1H, J=7.8 Hz), 8.38 (s, 1H), 8.12 (d, 1H, J=8.1 Hz), 8.08 (d, 1H, J=9.0 Hz), 7.91 (dd, 1H, J=7.9 Hz, J=7.9 Hz), 7.57 (d, 2H, J=8.6 Hz), 7.41 (d, 1H, J=2.3 Hz), 7.28-7.21 (m, 3H), 5.61 (s, 2H), 3.03 (s, 3H). (ESI) m/z 558 (M+H)⁺.

3-({5-[5-fluoro-3-(trifluoromethyl)phenyl](1,2,4-oxadiazol-3-yl)}methoxy)-3-{4-[(methylsulfonyl)amino]phenyl}chromen-4-one; ¹H NMR (300 MHz, DMSO-d₆) δ: 9.85 (s, 1H), 8.49 (s, 1H), 8.33-8.08 (m, 4H), 7.56 (d, 2H, J=8.7 Hz), 7.42-7.22 (m, 4H), 5.62 (s, 2H), 3.02 (s, 3H). (ESI) m/z 576.1 (M+H)⁺.

3-[(3-{4-[(methylsulfonyl)amino]phenyl}-4-oxochromen-7-yloxy)methyl]-benzenecarbonitrile; ¹H NMR (400 MHz, DMSO-d₆) d: 9.84 (s, 1H), 8.47 (s, 1H), 8.07 (d, 1H, J=9.2 Hz), 8.00 (s, 1H), 7.86 (d, 2H, J=7.6 Hz), 7.66 (dd, 1H, J=7.6, 7.6 Hz), 7.57 (d, 2H, J=8.8 Hz), 7.31-7.20 (m, 4H), 5.36 (s, 2H), 3.03 (s, 3H). (ESI) m/z 447 (M+H)⁺.

3-{[3-(4-methylsulfonylaminophenyl)-4-oxochromen-7-yloxy]methyl}benzamide; ¹H NMR (400 MHz, DMSO-d₆) δ: 9.83 (s, 1H), 8.46 (s, 1H), 8.06 (d, 1H, J=8.9 Hz), 8.01 (s, 2H), 7.87 (d, 1H, J=7.5 Hz), 7.65 (d, 1H, J=7.9 Hz), 7.57 (d, 2H, J=8.6 Hz), 7.50 (dd, 1H, J=7.7, 7.7 Hz), 7.40 (br s, 1H), 7.30 (d, 1H, J=2.2 Hz), 7.26 (d, 2H, J=8.6 Hz), 7.19 (dd, 1H, J=2.2, 8.9 Hz), 5.33 (s, 2H), 3.02 (s, 3H). (ESI) m/z 465 (M+H)⁺.

### EXAMPLE 9

### Preparation of a Compound of Formula I

A. Preparation of a Compound of Formula I in which R¹ is 3-Benzoic acid, R is 4-[(Methylsulfonyl)amino, R³ is Hydrogen, X Y and Z are —CH—, V is Oxygen, and W is Methylene

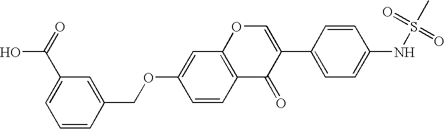

To a solution of prop-2-enyl 3-[(3-{4-[(methylsulfonyl)amino]phenyl}-4-oxochromen-7-yloxy)methyl]benzoate (88.8 mg, 0.176 mmol), tetrakis(triphenyl-phosphine)palladium(0) (10 mg, 0.009 mmol) in dry tetrahydrofuran 2 ml) was added morpholine (77 mg, 0.88 mmol), and the mixture was stirred at room temperature under argon for 2 hours. Solvent was then removed reduced pressure, and the residue dissolved in acetone, mixed with silica gel, the solvent removed under reduced pressure, and the silica gel eluted with methylene chloride/methanol (95/5) containing 1% acetic acid, to provide 3-[(3-{4-[(methylsulfonyl)amino]phenyl}-4-oxochromen-7-yloxy)methyl]benzoic acid ((67.8 mg); ¹H NMR (400 MHz, DMSO-d₆) d: 13.1 (br s, 1H), 9.84 (s, 1H), 8.47 (s, 1H), 8.08-8.06 (m, 2H), 7.94 (d, 1H, J=7.8 Hz), 7.76 (d, 1H, J=7.7 Hz), 7.58-7.45 (m, 3H), 7.30 (d, 1H, J=1.8 Hz), 7.27 (d, 2H, J=8.5 Hz), 7.20 (dd, 1H, J=1.8 Hz, J=8.9 Hz), 5.38 (s, 2H), 3.03 (s, 3H). (ESI) m/z 466 (M+H).

B. Preparation of a Compound of Formula I in which R¹ is 3-Benzoic acid, R³ is Hydrogen, X Y and Z are —CH—, V is Oxygen, and W is Methylene Varying R²

Similarly, replacing prop-2-enyl 3-[(3-{4-[(methylsulfonyl)amino]phenyl}-4-oxochromen-7-yloxy)methyl]benzoate with other compounds of Formula I in which R¹ is prop-2-enylbenzoate, and following the procedure of 9A above, the following compounds of Formula I in which R¹ is benzoic acid were prepared:

3-{[3-(4-hydroxyphenyl)-4-oxochromen-7-yloxy]methyl}benzoic acid; ¹H NMR (400 MHz, DMSO-d₆) δ: 13.1 (br s, 1H), 9.59 (br s, 1H), 8.38 (s, 1H), 8.08 (s, 1H), 8.05 (d, 1H, J=9.0 Hz), 7.94 (d, 1H, J=7.8 Hz), 7.75 (d, 1H, J=7.7 Hz), 7.56 (dd, 1H, J=7.5 Hz, J=7.8 Hz), 7.40 (d, 2H, J=8.7 Hz), 7.29 (d, 1H, J=1.9 Hz), 7.18 (dd, 1H, J=1.9 Hz, J=9.0 Hz), 6.82 (d, 2H, J=8.7 Hz), 5.37 (s, 2H). (ESI) m/z 389 (M+H).

3-(3-{[3-(4-hydroxyphenyl)-4-oxochromen-7-yloxy]methyl}-1,2,4-oxadiazol-5-yl)benzoic acid; ¹H NMR (400 MHz, DMSO-d₆) δ: 13.5 (s, 1H), 9.54 (br s, 1H), 8.62 (s, 1H), 8.40 (s, 1H), 8.36 (d, 1H, J=7.7 Hz), 8.25 (d, 1H, J=7.8 Hz), 8.08 (d, 1H, J=8.9 Hz), 7.79 (dd, 1H, J=7.8 Hz, J=7.8 Hz), 7.42-7.40 (m, 3H), 7.23 (dd, 1H, J=1.6 Hz, J=9.0 Hz), 6.82 (d, 2H, J=8.4 Hz), 5.59 (s, 2H). (ESI) m/z 457 (M+H).

3-{[3-(4-aminophenyl)-4-oxochromen-7-yloxy]methyl}benzoic acid; (ESI) m/z 388 (M+H)⁺.

### EXAMPLE 10

### Preparation of a Compound of Formula I

A. Preparation of a Compound of Formula I in which R¹ is 3-Methylbenzoate, R² is 4-[(Methylamino)carbonylamino R³ is Hydrogen, X Y and Z are —CH—, V is Oxygen, and W is Methylene

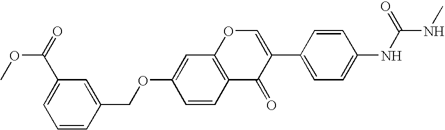

A suspension of methyl 3-{[3-(4-aminophenyl)-4-oxochromen-7-yloxy]methyl}benzoate (100 mg, 0.25 mmol) and methyl isocyanate (57 mg) in tetrahydrofuran (1 ml) was placed in a sealed tube, and the mixture stirred at room temperature for 3 days. The reaction mixture was slurried with methylene chloride, and solvent removed under reduced pressure, to provide crude methyl 3-[(3-{4-[(methylamino)carbonylamino]phenyl}-4-oxochromen-7-yloxy)methyl]benzoate. The solid was dissolved in a mixture of methanol/methylene chloride, mixed with silica gel, solvent removed, and the silica gel eluted with methanol/methylene chloride (3/97) to provide 90 mg of pure product. (ESI) m/z 459 (M+H)⁺.

B. Preparation of a Compound of Formula I in which R¹ is 3-Methylbenzoaate, R² is 4-acetylamino R³ is Hydrogen, X Y and Z are —CH—, V is Oxygen, and W is Methylene

Similarly, replacing methyl isocyanate by acetyl chloride, and following the procedure of 10A above, methyl 3-({3-[4-(acetylamino)phenyl]-4-oxochromen-7-yloxy}methyl)benzoate was prepared.

### EXAMPLE 11

### Preparation of a Compound of Formula I

A. Preparation of a Compound of Formula I in which R¹ is 2-[4-(4-methoxyphenyl)piperazinyl], R² is 4-Hydroxy R³ is Hydrogen, X Y and Z are —CH—, V is Oxygen, and W is Ethylene

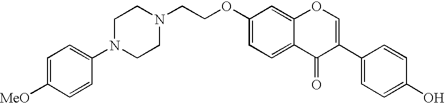

### Step 1

1-(4-methoxyphenyl)piperazine was dissolved in N,N-dimethylformamide, and potassium carbonate and 1-bromo-2-chloroethane were added. The resulting mixture was stirred at room temperature overnight, the solid material filtered off, and the solvent removed from the filtrate under reduced pressure. The residue was purified by biotage chromatography eluting with 3:7 ethyl acetate:hexanes, to provide 1-[4-(2-chloroethyl)piperazinyl]-4-methoxybenzene.

### Step 2

To a solution of 1-[4-(2-chloroethyl)piperazinyl]-4-methoxybenzene (0.929 mmol) and 4,7-dihydroxyisoflavone (0.929 mmol) in acetone (10 ml)was added 11% potassium hydroxide (0.5 ml), and the mixture stirred at reflux temperature for 48 hours. Sufficient methanol was added to precipitate unreacted starting material, which was filtered off, and solvent was removed from the filtrate under reduced pressure. The residue was purified by biotage column chromatography, eluting with 5% methanol/methylene chloride, to provide pure 3-(4-hydroxyphenyl)-7-{2-[4-(4-methoxyphenyl)piperazinyl]ethoxy}chromen-4-one.

### B.

Similarly, the following piperazinyl derivatives were prepared:
7-{2-[4-(4-fluorophenyl)piperazinyl]ethoxy}-3-(4-hydroxyphenyl)chromen-4-one;
3-(4-hydroxyphenyl)-7-(2-piperazinylethoxy)chromen-4-one;
N-(3-fluorophenyl)(4-{2-[3-(4-hydroxyphenyl)-4-oxochromen-7-yloxy]ethyl}-piperazinyl)carboxamide;
7-[2-(4-{[(3-fluorophenyl)amino]thioxomethyl}piperazinyl)ethoxy]-3-(4-hydroxyphenyl)chromen-4-one;
N-(2,4-difluorophenyl)(4-{2-[3-(4-hydroxyphenyl)-4-oxochromen-7-yloxy]ethyl}piperazinyl)carboxamide;

### EXAMPLE 12

### Preparation of a Compound of Formula I

A. Preparation of a Compound of Formula I in which R¹ is 2-[3-fluoro-5-(trifluoromethyl)phenyl]-1,3-oxazole], R² is 4-Hydroxy, R³ is Hydrogen, X Y and Z are —CH—, V is Oxygen, and W is Ethylene

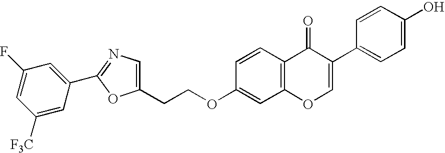

### Step 1

In a 50 mL round bottomed flask was placed diethyl malonate (3.72 g, 23.25 mmol, 5 equiv.) and N,N-dimethylformamide (10 mL). To the solution was added sodium hydride (60% suspension in mineral oil, 744.0 mg, 18.6 mmol, 4.0 equiv.) at room temperature portionwise over 10 minutes. After stirring for 30 minutes a solution of 4-(chloromethyl)-2-[5-fluoro-3-(trifluoromethyl)phenyl]-1,3-oxazole (1.30 g, 4.65 mmol) in N,N-dimethylformamide (10 mL) was added at 0° C. over 15 minutes, and the reaction mixture allowed to warm up to ambient temperature. To the mixture was added sodium iodide (697.0 mg, 4.65 mmol, 1 equiv) at room temperature. The reaction mixture was stirred at the same temperature for 2 hours. Water was then added to the reaction mixture (30 mL) and the whole was extracted with ethyl acetate (30 mL×3). The organic layers were combined, washed with brine (30 mL) and dried with sodium sulfate. After removal of the solvent under reduced pressure the crude mixture was purified by a silica-gel column chromatography (SiO₂=80 g, hexane:EtOAc=7:1) repeatedly. The desired product, diethyl 2-({2-[5-fluoro-3-(trifluoromethyl)phenyl]-1,3-oxazol-4-yl}methyl)propane-1,3-dioate, was obtained as colorless powder (1.75 g).

### Step 2

The product of Step 1 was used without further purification. The product (606.7 mg, 1.50 mmol) was placed in a 50 mL round bottomed flask, and lithium chloride (127.6 mmol, 3.01 mmol, 2 equiv.), dimethylsulfoxide (5 mL) and water (0.5 mL) added, and the mixture heated at 190-195° C. for 3 hours. To the reaction mixture was added water (30 mL) and the whole was extracted with ethyl acetate (30 mL×3). The combined organic layers were washed with brine (30 mL) and dried over sodium sulfate. After removal of the solvent under reduced pressure the crude mixture was purified by a silica-gel column chromatography (SiO₂=80 g, hexane:EtOAc=3:1). The desired product, ethyl 3-{2-[5-fluoro-3-(trifluoromethyl)phenyl]-1,3-oxazol-4-yl}propanoate, was obtained as light yellow oil (345.5 mg).

### Step 3

The product of Step 2 (330.0 mg, 0.996 mmol) was placed in a 250 mL round bottomed flask and dissolved in tetrahydrofuran (3 mL). The solution was treated with lithium aluminum hydride at 0° C. under nitrogen atmosphere. After stirring for 30 minutes, Celite (3 g) was added to the reaction mixture, followed by methanol (5 mL) and water (3 mL) successively. The resulting suspension was filtered through a glass filter, and the residue on the filter washed with ethyl acetate (50 mL). The solvent was removed under reduced pressure to give a colorless oil (298.3 mg). The crude mixture was purified by a silica-gel column chromatography (SiO₂=80 g, hexane:EtOAc=7:1) to give 3-{2-[5-fluoro-3-(trifluoromethyl)phenyl]-1,3-oxazol-4-yl}propan-1-ol as a colorless oil (255.3 mg, 0.883 mmol, 89%).

### Step 4

To 3-{2-[5-fluoro-3-(trifluoromethyl)phenyl]-1,3-oxazol-4-yl}propan-1-ol (250.3 mg, 0.865 mmol) was added a mixture of triphenyl phosphate (295.4 mg, 0.952 mmol, 1.1 equiv.) and methyl iodide (184.2 mg, 1.298 mmol, 1.5 equiv.). The mixture was heated at 130° C., while adding a further amount of methyl iodide (184.2 mg, 1.298 mmol, 1.5 equiv.). The reaction mixture was heated for a total of 2 hours, and then purified by column-chromatography (SiO₂=25 g, hexane/EtOAc=7:1) followed by preparative TLC (SiO₂=6 plates, hexane/EtOAc=15:1) to give 2-[5-fluoro-3-(trifluoromethyl)phenyl]-4-(3-iodopropyl)-1,3-oxazole (116.1 mg, 0.291 mmol, 34%) as a colorless oil.

### Step 5

4′,7-Dihydroxyisoflavone (31.3 mg, 0.123 mmol), 2-[5-fluoro-3-(trifluoromethyl)phenyl]-4-(3-iodopropyl)-1,3-oxazole (48.9 mg, 0.123 mmol, 1.0 equiv.) and cesium carbonate (40.0 mg, 0.123 mmol, 1.0 equiv) were placed in a 25 mL flask. To the flask was added dimethylsulfoxide (3 mL) at room temperature to dissolve the starting materials, and the reaction mixture stirred room temperature for 15 hours. To the mixture were added water (30 mL) and the whole was extracted with ethyl acetate (30 mL×3). The combined organic layers were washed with brine (30 mL) and dried with sodium sulfate to give a crude mixture as colorless oil (64.2 mg). The crude mixture was purified by column-chromatography (SiO₂=80 g, hexane/EtOAc=2:1 to 1:1) to give 7-(2-{2-[3-fluoro-5-(trifluoromethyl)phenyl] (1,3-oxazol-5-yl)}ethoxy)-3-(4-hydroxyphenyl)chromen-4-one (49.1 mg, 0.0934 mmol, 76%) as colorless crystals.

Similarly prepared was 7-(3-{2-[3-fluoro-5-(trifluoromethyl)phenyl] (1,3-oxazol-4-yl)}propoxy)-3-(4-hydroxyphenyl)chromen-4-one.

### EXAMPLE 13

### Preparation of a Compound of Formula I

A. Preparation of a Compound of Formula I in which R¹ is 4-Fluorophenyl R² is 4-Hydroxy R³ is Hydrogen, X Y and Z are —CH—, V is Oxygen, and W is —C(O)CH₂—

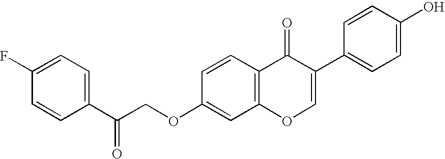

Dihydroxyisoflavone (0.2 g, 0.78 mmol) was suspended in acetone (10 ml), and to this suspension was added 2-bromo-1-(4-fluorophenyl)ethan-1-one (0.16 g, 0.75 mmol) and 11% potassium hydroxide (0.78 mmol). The mixture was refluxed for 24 hours, and the solvent removed under reduced pressure. The residue was treated with water, sonicated, filtered, and air-dried. The solid was triturated with methanol, filtered, to afford 7-[2-(4-fluorophenyl)-2-oxoethoxy]-3-(4-hydroxyphenyl)chromen-4-one. If desired, the product may be further purified by preparative thin layer chromatography, eluting with dichloromethane/methanol 15/1.

### B.

Similarly, following the procedures of Example 13A above, replacing 2-bromo-1-(4-fluorophenyl)ethan-1-one with other haloacetophone derivatives, the following compounds were prepared:
7-[2-(3-fluorophenyl)-2-oxoethoxy]-3-(4-hydroxyphenyl)chromen-4-one;
3-(4-hydroxyphenyl)-7-{2-oxo-2-[2-(trifluoromethyl)phenyl]ethoxy}chromen-4-one;
3-(4-hydroxyphenyl)-7-{2-oxo-2-[2-(trifluoromethyl)phenyl]ethoxy}chromen-4-one.

### EXAMPLE 14

### Preparation of a Compound of Formula I

A. Preparation of a Compound of Formula I in which R¹ is 3-Trifluoromethylphenyl, R² is 4-Hydroxy R³ is Hydrogen, X, Y and Z are —CH—, V is Oxygen, and W is —NHC(O)CH₂—

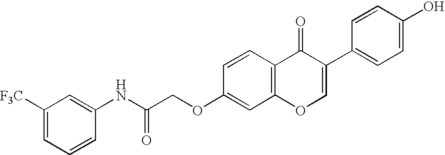

Dihydroxyisoflavone (0.2 g, 0.78 mmol) was suspended in acetone (10 ml), and to this suspension was added 2-chloro-N-[3-(trifluoromethyl)phenyl]acetamide (0.18 g, 0.78 mmol) and 11% potassium hydroxide (0.78 mmol). The mixture was refluxed for 24 hours, and the solvent removed under reduced pressure. The residue was treated with water, sonicated, filtered, and air-dried. The solid was triturated with methanol, filtered, to afford 2-[3-(4-hydroxyphenyl)-4-oxochromen-7-yloxy]-N-[3-(trifluoromethyl)phenyl]acetamide. If desired, the product may be further purified by preparative thin layer chromatography, eluting with dichloromethane/methanol 15/1.

### B.

Similarly, following the procedures of Example 14A above, replacing 2-chloro-N-[3-(trifluoromethyl)phenyl]acetamide with other haloacetaamide derivatives, the following compounds were prepared:
N-[(1S)-1-(4-fluorophenyl)ethyl]-2-[3-(4-hydroxyphenyl)-4-oxochromen-7-yloxy]acetamide;
2-[3-(4-hydroxyphenyl)-4-oxochromen-7-yloxy]-N-[2-(trifluoromethyl)phenyl]acetamide;
N-(3-fluorophenyl)-2-[3-(4-hydroxyphenyl)-4-oxochromen-7-yloxy]acetamide;
N-[(1R)-1-(4-fluorophenyl)ethyl]-2-[3-(4-hydroxyphenyl)-4-oxochromen-7-yloxy]acetamide.

### EXAMPLE 15

### Preparation of a Compound of Formula I

A. Preparation of a Compound of Formula I in which R¹ is 3-Trifluoromethylphenyl, R² is 4-Hydroxy R³ is Hydrogen, X Y and Z are —CH—, V is Oxygen, and W is —CH₂NHCH₂CH(OH)CH₂—

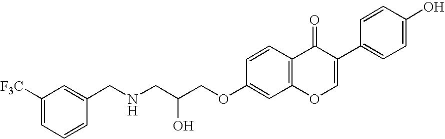

### Step 1

A mixture of 7-hydroxy-3-(4-methoxyphenyl)chromen-4-one (0.86 g, 3.21 mmol), epichlorohydrin (1.25 ml, 16 mmol) and potassium carbonate (0.89 g, 6.42 mmol) in dimethylformamide (20 ml) was stirred at 80° C. for 3 hours. After removing solvent under reduced pressure, water was added to the residue, and the precipitate filtered off and washed with water. The crude product was purified by chromatography on silica gel, eluting with ethyl acetate/hexanes (1:4 to 2:3), to afford 3-(4-methoxyphenyl)-7-(oxiran-2-ylmethoxy)chromen-4-one.

### Step 2

3-(4-Methoxyphenyl)-7-(oxiran-2-ylmethoxy)chromen-4-one (0.24 g, 0.74 mmol), 3-(trifluoromethyl)benzylamine (0.11 ml, 0.74 mmol) and diisopropylethylamine (0.26 g, 1.47 mmol) was stirred in ethanol (15 ml) at 78° C. overnight. The solvent was removed under reduced pressure, and the residue chromatographed on silica gel, eluting with 5% methanol/dichloromethane, followed by recrystallization from ethyl acetate/hexane to provide 7-[2-hydroxy-3-({[3-(trifluoromethyl)phenyl]methyl}amino)propoxy]-3-(4-methoxyphenyl)chromen-4-one.

### Step 3

To a stirred suspension of 7-[2-hydroxy-3-({[3-(trifluoromethyl)phenyl]methyl}-amino)propoxy]-3-(4-methoxyphenyl)chromen-4-one (38 mg, 0.076 mmol) in methylene chloride at ° C. was added boron tribromide (1M, 0.38 ml). The resulting mixture was stirred at room temperature for 4 hours, then the solvent removed under reduced pressure. The residue was purified by preparative thin layer chromatography, eluting with 10% methanol/dichloromethane, to provide 3-(4-hydroxyphenyl)-7-[2-hydroxy-3-({[3-(trifluoromethyl)phenyl]methyl}amino)propoxy]chromen-4-one.

### B.

Similarly, following the procedures of Example 15A above, but substituting 3-(trifluoromethyl)benzylamine by 3,5-difluorobenzylamine, the following compound was prepared:
7-(3-{[(3,5-difluorophenyl)methyl]amino}-2-hydroxypropoxy)-3-(4-hydroxyphenyl)chromen-4-one; and
7-(2-{[(4-fluorophenyl)ethyl]amino}ethoxy)-3-(4-hydroxyphenyl)chromen-4-one.

### EXAMPLE 16

### Preparation of a Compound of Formula I

A. Preparation of a Compound of Formula I in which R¹ is Phenyl R² is 4-Hydroxy, R³ is Hydrogen, X Y and Z are —CH—, V is Oxygen, and W is —CH₂CH(OH)CH)₂—

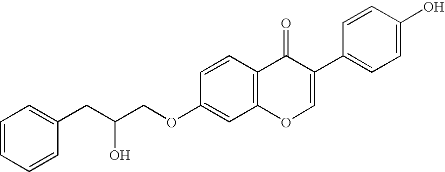

### Step 1

To a solution of cuprous iodide (0.14 g, 0.74 mmol) in tetrahydrofuran (2 ml) was added phenylmagnesium bromide in tetrahydrofuran (1M, 2.22 ml, 2.22 mmol) dropwise at −40° C. After 5 minutes 3-(4-methoxyphenyl)-7-(oxiran-2-ylmethoxy)chromen-4-one (0.24 g, 0.74 mmol) in tetrahydrofuran (4 ml) was added slowly, and stirred at −40° C. for 1 hour. The mixture was quenched with saturated ammonium chloride and water, extracted with ethyl acetate, the organic layer washed with brine, dried over sodium sulfate, and the solvent removed under reduced pressure. The residue was purified by preparative thin layer chromatography, eluting with 10% methanol/methylene chloride, followed by ethyl acetate/hexane 2/3, to provide 7-(2-hydroxy-3-phenylpropoxy)-3-(4-methoxyphenyl)chromen-4-one.

### Step 2

The product of step 1 was then reacted with boron tribromide as shown in Example 15, step 3, to provide 3-(4-hydroxyphenyl)-7-(2-hydroxy-3-phenylpropoxy)chromen-4-one.

### EXAMPLE 17

### Preparation of a Compound of Formula I

A. Preparation of the R Enantiomer of a Compound of Formula I in which R¹ is 3-[5-Fluoro-3-(trifluoromethyl)phenyl](1,2,4-oxadiazol-5-yl), R² is 4-Hydroxy R³ is Hydrogen, X Y and Z are —CH—, V is Oxygen, and W is —CH(CH₃)—

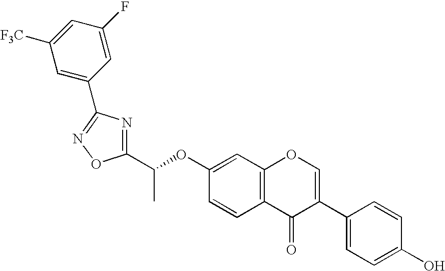

### Step 1

A solution of [5-fluoro-3-(trifluoromethyl)-phenyl](hydroxyimino)methylamine (28.04 g, 126.24 mmol), prepared as shown in Example 1, was dissolved in tetrahydrofuran (40 ml) and cooled to −78° C. A solution of(1S)-1-(chlorocarbonyl)ethyl acetate (20 g, 128.82 mmol) in tetrahydrofuran (20 ml) was added dropwise under an atmosphere of dry nitrogen, and stirred for 10 minutes after the addition was complete. A solution of diisopropylethylamine (27.0 ml, 155 mmol) was then added dropwise, and the reaction mixture allowed to warm to room temperature. The mixture was stirred for two hours, then the solvent removed under reduced pressure. The residue was poured into ethyl acetate (150 ml), washed with water (2×50 ml), brine (2×50 ml), and dried over sodium sulfate. Solvent was removed under reduced pressure, to provide 2-amino-2-[3-fluoro-5-(trifluoromethyl)phenyl]-1-azavinyl (2S)-2-acetyloxypropanoate as a pale yellow oil (39.04 g, MS m/z 337.1 (M+H), which was used in the next reaction with no further purification.

### Step 2

To a solution of 2-amino-2-[3-fluoro-5-(trifluoromethyl)phenyl]-1-azavinyl (2S)-2-acetyloxypropanoate (5.19 g, 15.43 mmol) in anhydrous tetrahydrofuran (20 ml) at 0° C. was added a solution of IM tetrabutylammonium fluoride in tetrahydrofuran (3 ml) dropwise under nitrogen. The reaction mixture was stirred for 3 hours at 0° C., then poured into ethyl acetate (50 ml), washed with water (2×20 ml), brine (30 ml) and dried over sodium sulfate. Solvent was removed under reduced pressure, and the residue purified by flash chromatography, eluting with methylene chloride, to provide (1S)-1-{3-[5-fluoro-3-(trifluoromethyl)phenyl](1,2,4-oxadiazol-5-yl)}ethyl acetate, LCMS 319.1.

### Step 3

To a solution of (1S)-1-{3-[5-fluoro-3-(trifluoromethyl)phenyl] (1,2,4-oxadiazol-5-yl)}ethyl acetate (900 mg, 2.83 mmol) in methanol (4 ml) at −15° C. was added an aqueous solution of potassium carbonate (10M, 10 ml). The mixture was stirred for 20 minutes, and the mixture allowed to warm to room temperature, stirring for 1 hour. The mixture was extracted with ethyl acetate (3×20 ml), and the combined organic phase washed with water (10 ml), brine (2×20 ml). Removal of the solvent under reduced pressure provided (1S)-1-{3-[5-fluoro-3-(trifluoromethyl)phenyl](1,2,4-oxadiazol-5-yl)}ethan-1-ol, which was crystallized from hexane to yield a white solid, LCMS 277.2.

### Step 4

To a solution of triphenylphosphine (262 mg, 1 mmol) in anhydrous tetrahydrofuran (15 ml) at −78° C. was added dropwise 40% diethylazodicarboxylate (0.45 ml, 1 mmol) in toluene, and the mixture stirred for 30 minutes at −78° C. A solution of dihydroxyisoflavone (300 mg, 1.14 mmol) in a mixture of tetrahydrofuran (8 ml) and N,N-dimethylformamide (3 ml) was added slowly, and the mixture stirred for 10 minutes. A solution of (1S)-1-{3-[5-fluoro-3-(trifluoromethyl)phenyl] (1,2,4-oxadiazol-5-yl)}ethan-1-ol (277 mg, 1 mmol) in tetrahydrofuran (8 ml) was added dropwise, the mixture stirred at −78° C. for 3 hours, and then allowed to warm to room temperature, stirring for 36 hours.

The reaction mixture was poured into ethyl acetate (40 ml), washed with water (10 ml), brine (2×10 ml), dried over sodium sulfate, and the solvent removed under reduced pressure. A mixture of dichloromethane/tetrahydrofuran (4 ml/1 ml) was added to the yellow residue, and the soluble portion was flash chromatographed over silica gel, eluting with ethyl acetate (0-30%)/hexane, to give a white solid, which was further purified by preparative thin layer chromatography, eluting with acetonitrile (2.5 97.5%/water, to provide 7-((1R)-1-{3-[5-fluoro-3-(trifluoromethyl)phenyl](1,2,4-oxadiazol-5-yl)}ethoxy)-3-(4-hydroxyphenyl)chromen-4-one; 245 mg, 0.48 mmol, 48%). MS m/z 513.1 (M+H), anal HPLC >99%, Chiralcel OJ-RH hplc 99.2% e.e. (mass detector), and 99.0% e.e. (UV detector) in acetonitrile/water.

1H NMR (400 MHz; CDCl₃) δ8.25 (d, 1H, J=9.0 Hz); 8.18 (s, 1H); 7.99 (m, 1H); 7.91 (s, 1H); 7.49 (m, 1H); 7.42 (d, 2H, J=8.6 Hz); 7.09 (dd, 1H, J=9.0, 2.3 Hz); 6.97 (d, 1H, J=2.3 Hz); 6.88 (d, 2H, J=9.0 Hz); 5.59 (t, 1H, J=6.6 Hz); 1.96 (d, 1H, J=6.6 Hz).

### EXAMPLE 18

### Preparation of a Prodrug of a Compound of Formula I

A. Preparation of the Phosphate of a Compound of Formula I in which R¹ is 5-Fluoro-3-(trifluoromethyl)phenyl](1,2-oxazol-5-yl), R² is 4-Hydroxy R³ is Hydrogen, X, Y and Z are —CH—, V is Oxygen, and W is CH₂—

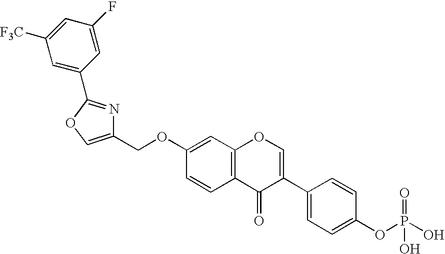

### Step 1

To a solution of 7-({2-[5-fluoro-3-(trifluoromethyl)phenyl](1,3-oxazol-4-yl)}methoxy)-3-(4-hydroxyphenyl)chromen-4-one (1 g, 2.01 mmol) in tetrahydrofuran (50 mL) was added 1-H-tetrazole (3% wt in acetonitrile, 65 ml, 22.1 mmol), followed by di-tert-butyl N,N-diethylphosphoramidite (2.57 ml, 4.6 mmol). After stirring at room temperature for 2 hours, the reaction mixture was diluted with methylene chloride and washed with saturated sodium bicarbonate. The organic layer was separated, and the aqueous layer extracted twice more with methylene chloride. The combined extracts were dried over sodium sulfate, and solvent removed under reduced pressure. The residue was purified by biotage column chromatography, eluting with ethyl acetate/hexane mixture (1:4) to afford 3-{4-[bis(tert-butoxy)phosphinooxy]phenyl}-7-({2-[5-fluoro-3-(trifluoromethyl)phenyl](1,3-oxazol-4-yl)}methoxy)chromen-4-one.

### Step 2

To a solution of the product of step 1, 3-{4-[bis(tert-butoxy)phosphinooxy]phenyl}-7-({2-[5-fluoro-3-(trifluoromethyl)phenyl](1,3-oxazol-4-yl)}methoxy)chromen-4-one, in a mixture of tetrahydrofuran (20 mL) and acetonitrile (10 mL) was added 6 mL of tert-butyl hydroperoxide in decane (5M-6M). The reaction mixture was stirred at room temperature for 1 hour, chilled in an ice bath, and 50 mL of 5% sodium bisulfite was added. The resulting mixture was stirred for 15 minutes, after which the ice bath was removed. The mixture was extracted with methylene chloride, the organic extract dried over sodium sulfate, and solvent removed under reduced pressure. The residue was purified by biotage column chromatography, eluting with 1:1 ethyl Acetate/hexanes mixture, to afford ditert-butyl 4-[7-({2-[5-fluoro-3-(trifluoromethyl)phenyl](1,3-oxazol-4-yl)}methoxy)-4-oxochromen-3-yl]phenyl phosphate.

### Step 3

To a solution of 3-{4-[bis(tert-butoxy)phosphinooxy]phenyl}-7-({2-[5-fluoro-3-(trifluoromethyl)phenyl](1,3-oxazol-4-yl)}methoxy)chromen-4-one prepared in Step 2 in methylene chloride (60 ml) was added trifluoroacetic acid (0.15 ml, 1.99 mmol). The reaction mixture was stirred at room temperature overnight, the solid filtered off, and washed with methylene chloride, to afford 100% pure (by HPLC) 4-[7-({2-[5-fluoro-3-(trifluoromethyl)phenyl](1,3-oxazol-4-yl)}methoxy)-4-oxochromen-3-yl]phenyl dihydrogen phosphate.

### EXAMPLE 19

### Preparation of a Prodrug of a Compound of Formula I

A. Preparation of the Methyldihydrogenphosphate of a Compound of Formula I in which R¹ is 5-Fluoro-3-(trifluoromethyl)phenyl](1,2-oxazol-5-yl), R² is 4-Hydroxy, R³ is Hydrogen, X Y and Z are —CH—, V is Oxygen, and W is CH₂—

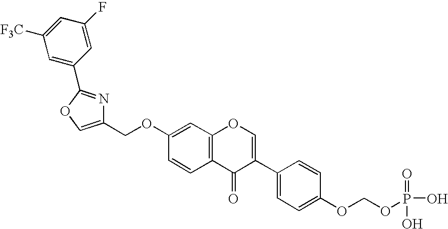

Step 1—Preparation of di-t-butyl Chloromethyl Phosphate

A 100 mL round bottomed flask was charged with potassium ditert-butyl phosphate (1.0 g, 4.03 mmol), sodium bicarbonate (677.4 mg, 8.06 mmol), n-butylammonium sulfate (68.2 mg, 0.403 mmol), water (10 ml) and methylene chloride (5 ml). To the mixture was added a solution of chloromethylchlorosulfonate (797.9 mg, 4.84 mmol) in methylene chloride (5 ml), and the mixture stirred at room temperature for 2 hours. To the reaction product was added water (30 ml), and the whole was extracted with methylene chloride (30 ml×3). The combined organic layers were washed with brine (30 ml), dried with Na₂SO₄, and solvent removed under reduced pressure. The residue was purified by column-chromatography (silica gel=80 g, hexane/ethyl acetate=1:1) to give di-t-butyl chloromethyl phosphate, as a colorless oil.

Step 2—Preparation of di-tert-butyl (4-(7-((2-(3-fluoro-5-(trifluoromethyl)phenyl)oxazol-4-yl)methoxy)-4-oxo-4H-chromen-3-yl)phenoxy)methyl Phosphate

In a 50 mL round bottomed flask 7-({2-[5-fluoro-3-(trifluoromethyl)phenyl](1,3-oxazol-4-yl)}methoxy)-3-(4-hydroxyphenyl)chromen-4-one (150.0 mg, 0.302 mmol) was treated with di-tert-butyl chloromethyl phosphate (156.2 mg, 0.604 mmol, 1.0 equiv) in the presence of potassium t-butoxide (67.8 mg, 0.604 mmol, 1.0 equiv) and sodium iodide (89.9 mg, 0.604 mmol, 1.0 equiv) in N,N-dimethylformamide (2 ml), and the mixture stirred at room temperature for 15 hours. To the mixture was added water (30 ml), and the whole was extracted with ethyl acetate (30 ml×3). The combined organic layers were washed with brine (30 ml), dried with Na₂SO₄, and solvent removed under reduced pressure, to give a crude mixture (345.1 mg). This mixture was purified by column-chromatography (SiO₂=80 g, hexane/EtOAc=1:1) to give di-tert-butyl {4-[7-({2-[5-fluoro-3-(trifluoromethyl)phenyl](1,3-oxazol-4-yl)}methoxy)-4-oxochromen-3-yl]phenoxy}methyl phosphate as a colorless oil.

Step 3—Preparation of (4-(7-((2-(3-fluoro-5-(trifluoromethyl)phenyl)oxazol-4-yl)methoxy)-4-oxo-4H-chromen-3-yl)phenoxy)methyl Dihydrogen Phosphate

In a 50 mL round bottomed flask ditert-butyl {4-[7-({2-[5-fluoro-3-(trifluoromethyl)phenyl](1,3-oxazol-4-yl)}methoxy)-4-oxochromen-3-yl]phenoxy}methyl phosphate (119.1 mg, 0.166 mmol) was treated with trifluoroacetic acid (37.9 mg, 0.332 mmol, 2.0 equiv) in methylene chloride (2 ml). The mixture was stirred at room temperature for 18 hours, methylene chloride (10 ml) added, and the suspension thus obtained was filtered through a glass filter. The residue on the filter was collected to give {4-[7-({2-[5-fluoro-3-(trifluoromethyl)phenyl](1,3-oxazol-4-yl)}methoxy)-4-oxochromen-3-yl]phenoxy}methyl dihydrogen phosphate.

### EXAMPLE 20

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules.

### EXAMPLE 21

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets.

### EXAMPLE 22

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### EXAMPLE 23

Tablets, each containing 30 mg of active ingredient, are prepared as follows:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in sterile water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### EXAMPLE 24

Suppositories, each containing 25 mg of active ingredient are made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### EXAMPLE 25

Suspensions, each containing 50 mg of active ingredient per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### EXAMPLE 26

A subcutaneous formulation may be prepared as follows:

| Ingredient | Quantity | |
|---|---|---|
| Active Ingredient | 5.0 | mg |
| Corn Oil | 1.0 | mL |

### EXAMPLE 27

An injectable preparation is prepared having the following composition:

| Ingredients | Amount | |
|---|---|---|
| Active ingredient | 2.0 | mg/ml |
| Mannitol, USP | 50 | mg/ml |
| Gluconic acid, USP | q.s. (pH 5-6) | |
| water (distilled, sterile) | q.s. to 1.0 | ml |

| | |
|---|---|
| Nitrogen Gas, NF | q.s. |

### EXAMPLE 28

A topical preparation is prepared having the following composition:

| Ingredients | grams |
|---|---|
| Active ingredient | 0.2-10 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60° C. with stirring. A sufficient quantity of water at 60° C. is then added with vigorous stirring to emulsify the ingredients, and water then added q.s. 100 g.

### EXAMPLE 29

Sustained Release Composition

| | Weight | Preferred | |
|---|---|---|---|
| Ingredient | Range (%) | Range (%) | Most Preferred |
| Active ingredient | 50-95 | 70-90 | 75 |
| Microcrystalline cellulose (filler) | 1-35 | 5-15 | 10.6 |
| Methacrylic acid copolymer | 1-35 | 5-12.5 | 10.0 |
| Sodium hydroxide | 0.1-1.0 | 0.2-0.6 | 0.4 |
| Hydroxypropyl methylcellulose | 0.5-5.0 | 1-3 | 2.0 |
| Magnesium stearate | 0.5-5.0 | 1-3 | 2.0 |

The sustained release formulations of this invention are prepared as follows: compound and pH-dependent binder and any optional excipients are intimately mixed(dry-blended). The dry-blended mixture is then granulated in the presence of an aqueous solution of a strong base which is sprayed into the blended powder. The granulate is dried, screened, mixed with optional lubricants (such as talc or magnesium stearate), and compressed into tablets. Preferred aqueous solutions of strong bases are solutions of alkali metal hydroxides, such as sodium or potassium hydroxide, preferably sodium hydroxide, in water (optionally containing up to 25% of water-miscible solvents such as lower alcohols).

The resulting tablets may be coated with an optional film-forming agent, for identification, taste-masking purposes and to improve ease of swallowing. The film forming agent will typically be present in an amount ranging from between 2% and 4% of the tablet weight. Suitable film-forming agents are well known to the art and include hydroxypropyl. methylcellulose, cationic methacrylate copolymers (dimethylaminoethyl methacrylate/methyl-butyl methacrylate copolymers—Eudragit® E—Röhm. Pharma), and the like. These film-forming agents may optionally contain colorants, plasticizers, and other supplemental ingredients.

The compressed tablets preferably have a hardness sufficient to withstand 8 Kp compression. The tablet size will depend primarily upon the amount of compound in the tablet. The tablets will include from 300 to 1100 mg of compound free base. Preferably, the tablets will include amounts of compound free base ranging from 400-600 mg, 650-850 mg, and 900-1100 mg.

In order to influence the dissolution rate, the time during which the compound containing powder is wet mixed is controlled. Preferably the total powder mix time, i.e. the time during which the powder is exposed to sodium hydroxide solution, will range from 1 to 10 minutes and preferably from 2 to 5 minutes. Following granulation, the particles are removed from the granulator and placed in a fluid bed dryer for drying at about 60° C.

### EXAMPLE 30

### MAO-A, MAO-B, and ALDH-2 Inhibition Assays

ALDH2 inhibition assays were performed in sodium phosphate buffer (50 mM, pH 7.4) containing 1.2 mM NAD⁺ (Sigma N7004), 1 nM hALDH2 (recombinant human mitochondria ALDH), various concentrations of test compounds (from 0.03 nM to 1000 nM), and 0.15 mM freshly prepared formaldehyde solution (Ladd Research 20295). Reactions were initiated by the addition of formaldehyde and rates were recorded by monitoring NADH formation in a temperature controlled (25° C.) FluoroMax-2 Fluorimeter with excitation and emission wavelengths set at 340 and 460 nm, respectively. Slow binding or irreversible inhibitors (inhibitors that exhibit time-dependent inhibition kinetics) were pre-incubated with enzyme in the presence of NAD for 5-15 min before the addition of aldehyde substrate. Normally, IC₅₀ values were determined by fitting concentration-inhibition data to sigmoidal dose-response curves with two-floating parameters: IC₅₀ and Hill coefficient. When tight binding situation was suspected, apparent Ki values were estimated by fitting dose-response data to Morrison's equation [Methods in Enzymology 63, 437-467, 1979].

MAO-A (Sigma M7316) and MAO-B (Sigma M7441) inhibition assays were conducted in 96-well micro-plates according to the method described by Zhou et al. [Analytical Biochemistry 253, 169-174, 1997]. Both assays were performed in potassium phosphate buffer (0.1 M, pH 7.5) containing 0.5 mM tyramine (Sigma T2879), 0.12 mM N-acetyl-3,7-dihydrophenoxazine (Amplex Red, Invitrogen A12222), 1.2 unit/ml horseradish peroxidase (Sigma P2088), 0.6 unit/ml MAO-A or 2.5 unit/ml MAO-B, and various concentrations of test compounds (0.1 nM to 10 uM). Reactions were initiated by the addition of a pre-mixed solution of tyramine, horseradish peroxidase and Amplex Red, and were allowed to proceed at 37° C. for 30 min. Enzyme activities were determined by measuring absorbance at 570 nm (SpectralMax Plate reader), absorbance of oxidized Amplex Red generated by this horseradish peroxidase-coupled reaction. Nonspecifically formed products were determined in the presence of 2 and 10 □M clorgyline and deprenyl for MAO-A and MAO-B, respectively. IC₅₀ values were determined by fitting concentration-inhibition data to sigmoidal dose-response curves with two-floating parameters: IC₅₀ and Hill coefficient.

Representative data for several compounds of the invention are presented in Table 1 below.

TABLE 1
ALDH-2 AND MAO INHIBITION

| | | IC₅₀ | | IC₅₀ | IC₅₀ |
|---|---|---|---|---|---|
| | COMPOUND | hALDH2 | | hMAO-A | hMAO-B |
| PT-1. | 4-[7-({5-[3-fluoro-5- | 17% | | | |
| | (trifluoromethyl)phenyl](1,2,4-oxadiazol-3- | inhibition | | | |
| | yl)}methoxy)-4-oxochromen-3- | at 1 μM | | | |
| | yl]benzenecarbonitrile; | | | | |
| PT-2. | 7-({3-[5-fluoro-3- | 43% | | No | 8% |
| | (trifluoromethyl)phenyl](1,2,4-oxadiazol-5- | inhibition | | inhibition | inhibition |
| | yl)}ethoxy)-3-(4-hydroxyphenyl)chromen-4- | at 1 μM | | up to 10 μM | at 10 μM |
| | one; | | | | |
| PT-3. | ethyl 3-[7-({5-[3-fluoro-5- | 22% | | | |
| | (trifluoromethyl)phenyl](1,2,4-oxadiazol-3- | inhibition | | | |
| | yl)}methoxy)-4-oxochromen-3-yl]benzoate; | at 1 μM | | | |
| PT-4. | 3-(4-hydroxyphenyl)-7-({4-methyl-2-[4- | 0.20 | μM | | |
| | (trifluoromethyl)phenyl](1,3-thiazol-5- | | | | |
| | yl)}methoxy)chromen-4-one; | | | | |
| PT-5. | 3-(4-{[(4- | 0.19% | | | |
| | methylphenyl)sulfonyl]amino}phenyl)-7-({4- | inhibition | | | |
| | methyl-2-[4-(trifluoromethyl)phenyl](1,3- | at 1 μM | | | |
| | thiazol-5-yl)}methoxy)chromen-4-one; | | | | |
| PT-6. | methyl 3-{[3-(6-methoxy(3-pyridyl))-4- | 16% | | | |
| | oxochromen-7-yloxy]methyl}benzoate; | inhibition | | | |
| | | at 1 μM | | | |
| PT-7. | methyl 3-({3-[4-(hydroxymethyl)phenyl]-4- | 75% | | | |
| | oxochromen-7-yloxy}methyl)benzoate; | inhibition | | | |
| | | at 1 μM | | | |
| PT-8. | 7-({3-[5-fluoro-3- | 57% | | | |
| | (trifluoromethyl)phenyl](1,2,4-oxadiazol-5- | inhibition | | | |
| | yl)}methoxy)-3-{4- | at 1 μM | | | |
| | [(methylsulfonyl)amino]phenyl}chromen-4-one; | | | | |
| PT-9. | 2-fluoro-5-[7-({5-[5-fluoro-3- | 25% | | | |
| | (trifluoromethyl)phenyl](1,2,4-oxadiazol-3- | inhibition | | | |
| | yl)}methoxy)-4-oxochromen-3- | at 1 μM | | | |
| | yl]benzenecarbonitrile; | | | | |
| PT-10. | ethyl 2-(3-{4- | 60% | | | |
| | [(ethoxycarbonyl)methoxy]phenyl}-4- | inhibition | | | |
| | oxochromen-7-yloxy)acetate; | at 1 μM | | | |
| PT-11. | 7-{[5-(4-fluorophenyl)(1,2,4-oxadiazol-3- | 0.02 | μM | No | 35% |
| | yl)]methoxy}-3-(4-hydroxyphenyl)chromen-4- | | | inhibition | inhibition |
| | one; | | | up to 10 μM | at 10 μM |
| PT-12. | 3-(4-hydroxyphenyl)-7-({2-[3- | 0.003 | μM | No | No |
| | (trifluoromethyl)phenyl](1,3-oxazol-4- | | | inhibition | inhibition |
| | yl)}methoxy)chromen-4-one; | | | up to 10 μM | at 10 μM |
| PT-13. | 7-({2-[5-fluoro-3-(trifluoromethyl)phenyl](1,3- | 0.02 | μM | No | No |
| | oxazol-4-yl)}methoxy)-3-(4- | | | inhibition | inhibition |
| | hydroxyphenyl)chromen-4-one; | | | up to 10 μM | at 10 μM |
| PT-14. | 7-{[2-(3,5-difluorophenyl)(1,3-oxazol-4- | 0.06 | μM | No | No |
| | yl)]methoxy}-3-(4-hydroxyphenyl)chromen-4- | | | inhibition | inhibition |
| | one; | | | up to 10 μM | at 10 μM |
| PT-15. | 7-{[2-(3,4-difluorophenyl)(1,3-oxazol-4- | 0.12 | μM | | |
| | yl)]methoxy}-3-(4-hydroxyphenyl)chromen-4- | | | | |
| | one; | | | | |
| PT-16. | 7-{[2-(4-fluorophenyl)(1,3-oxazol-4- | 0.047 | μM | No | No |
| | yl)]methoxy}-3-(4-hydroxyphenyl)chromen-4- | | | inhibition | inhibition |
| | one; and | | | up to 10 μM | at 10 μM |
| PT-17. | 7-{[2-(4-chlorophenyl)(1,3-oxazol-4- | 0.573 | μM | | |
| | yl)]methoxy}-3-(4-hydroxyphenyl)chromen-4- | | | | |
| | one. | | | | |
| PT-18. | 3-(4-hydroxyphenyl)-7-({2-[3- | 0.003 | μM | No | No |
| | (trifluoromethyl)phenyl](1,3-oxazol-4- | | | inhibition | inhibition |
| | yl)}methoxy)chromen-4-one; | | | up to 10 μM | at 10 μM |
| PT-19. | 7-({2-[5-fluoro-3-(trifluoromethyl)phenyl](1,3- | 0.02 | μM | No | No |
| | oxazol-4-yl)}methoxy)-3-(4- | | | inhibition | inhibition |
| | hydroxyphenyl)chromen-4-one; | | | up to 10 μM | at 10 μM |
| PT-20. | 7-{[2-(3,5-difluorophenyl)(1,3-oxazol-4- | 0.06 | μM | No | No |
| | yl)]methoxy}-3-(4-hydroxyphenyl)chromen-4- | | | inhibition | inhibition |
| | one; | | | up to 10 μM | at 10 μM |
| PT-21. | 7-{[2-(3,4-difluorophenyl)(1,3-oxazol-4- | 0.12 | μM | | |
| | yl)]methoxy}-3-(4-hydroxyphenyl)chromen-4- | | | | |
| | one; | | | | |
| PT-22. | 7-{[2-(4-fluorophenyl)(1,3-oxazol-4- | 0.047 | μM | No | No |
| | yl)]methoxy}-3-(4-hydroxyphenyl)chromen-4- | | | inhibition | inhibition |
| | one; and | | | up to 10 μM | at 10 μM |
| PT-23. | 7-{[2-(4-chlorophenyl)(1,3-oxazol-4- | 0.573 | μM | | |
| | yl)]methoxy}-3-(4-hydroxyphenyl)chromen-4- | | | | |
| | one. | | | | |
| PT-24. | 3-(4-hydroxyphenyl)-7-[(5-phenyl(1,2,4- | 0.16 | μM | No | No |
| | oxadiazol-3-yl))methoxy]chromen-4-one | | | inhibition | inhibition |
| | | | | up to 40 μM | up to 40 μM |
| PT-25. | 3-{[3-(4-hydroxyphenyl)-4-oxochromen-7- | 0.004 | μM | No | No |
| | yloxy]methyl}benzenecarbonitrile; | | | inhibition | inhibition |
| | | | | up to 10 μM | up to 10 μM |
| PT-26. | 3-(4-hydroxyphenyl)-7-{[3- | 0.034 | μM | | |
| | (trifluoromethyl)phenyl]methoxy}chromen-4- | | | | |
| | one; | | | | |
| PT-27. | 3-(4-hydroxyphenyl)-7-{[4-methoxy-3- | 0.02 | μM | No | No |
| | (trifluoromethyl)phenyl]methoxy}chromen-4- | | | inhibition | inhibition |
| | one; | | | up to 10 μM | up to 10 μM |
| PT-28. | 7-{[3-fluoro-5- | 0.058 | μM | | |
| | (trifluoromethyl)phenyl]methoxy}-3-(4- | | | | |
| | hydroxyphenyl)chromen-4-one; | | | | |
| PT-29. | 7-({5-[3-fluoro-5- | 0.01 | μM | No | No |
| | (trifluoromethyl)phenyl](1,2,4-oxadiazol-3- | | | inhibition | inhibition |
| | yl)}methoxy)-3-(4-hydroxyphenyl)chromen-4- | | | up to 30 μM | up to 30 μM |
| | one; | | | | |
| PT-30. | 7-({5-[4-fluoro-3- | 0.10 | μM | No | No |
| | (trifluoromethyl)phenyl](1,2,4-oxadiazol-3- | | | inhibition | inhibition |
| | yl)}methoxy)-3-(4-hydroxyphenyl)chromen-4- | | | up to 10 μM | up to 10 μM |
| | one; | | | | |
| PT-31. | 7-({5-[2,5-bis(trifluoromethyl)phenyl](1,2,4- | 0.02 | μM | No | No |
| | oxadiazol-3-yl)}methoxy)-3-(4- | | | inhibition | inhibition |
| | hydroxyphenyl)chromen-4-one; | | | up to 10 μM | up to 10 μM |
| PT-32. | prop-2-enyl 3-(3-{[3-(4-hydroxyphenyl)-4- | 1.15 | μM | No | No |
| | oxochromen-7-yloxy]methyl}-1,2,4-oxadiazol- | | | inhibition | inhibition |
| | 5-yl)benzoate; (ESI) m/z 497 (M + H)⁺. | | | up to 10 μM | up to 10 μM |
| PT-33. | methyl 3-{[3-(4-hydroxyphenyl)-4- | 0.15 | μM | No | 0.3 μM |
| | oxochromen-7-yloxy]methyl}benzoate; | | | inhibition | |
| | | | | up to 30 μM | |
| PT-34. | ethyl 4-{[3-(4-hydroxyphenyl)-4-oxochromen- | 0.13 | μM | 24 μM | 2.3 μM |
| | 7-yloxy]methyl}benzoate; | | | | |
| PT-35. | methylethyl 3-{[3-(4-hydroxyphenyl)-4- | 0.02 | μM | No | No |
| | oxochromen-7-yloxy]methyl}benzoate; | | | inhibition | inhibition |
| | | | | up to 10 μM | up to 10 μM |
| PT-36. | 4-{[3-(4-hydroxyphenyl)-4-oxochromen-7- | 0.17 | μM | No | No |
| | yloxy]methyl}benzoic acid; (ESI) m/z 389 (M + H)⁺. | | | inhibition | inhibition |
| | | | | up to 40 μM | up to 30 μM |
| PT-37. | 4-{[3-(4-hydroxyphenyl)-4-oxochromen-7- | 0.38 | μM | No | No |
| | yloxy]methyl}benzamide; | | | inhibition | inhibition |
| | | | | up to 30 μM | up to 30 μM |
| PT-38. | 3-(4-hydroxyphenyl)-7-({5-[4- | 0.6 | μM | No | No |
| | (trifluoromethyl)phenyl](1,2,4-oxadiazol-3- | | | inhibition | inhibition |
| | yl)}methoxy)chromen-4-one; | | | up to 30 μM | up to 30 μM |
| PT-39. | 7-({5-[3,5-bis(trifluoromethyl)phenyl](1,2,4- | 0.13 | μM | No | No |
| | oxadiazol-3-yl)}methoxy)-3-(4- | | | inhibition | inhibition |
| | hydroxyphenyl)chromen-4-one; | | | up to 10 μM | up to 10 μM |
| PT-40. | 3-(3-{[3-(4-hydroxyphenyl)-4-oxochromen-7- | 0.022 | μM | No | No |
| | yloxy]methyl}-1,2,4-oxadiazol-5- | | | inhibition | inhibition |
| | yl)benzenecarbonitrile; | | | up to 10 μM | up to 10 μM |
| PT-41. | 3-(3-{[3-(4-hydroxyphenyl)-4-oxochromen-7- | 0.01 | μM | No | No |
| | yloxy]methyl}-1,2,4-oxadiazol-5-yl)benzoic | | | inhibition | inhibition |
| | acid; | | | up to 10 μM | up to 10 μM |
| PT-42. | 7-{[5-(3-fluorophenyl)(1,2,4-oxadiazol-3- | 0.062 | μM | No | No |
| | yl)]methoxy}-3-(4-hydroxyphenyl)chromen-4- | | | inhibition | inhibition |
| | one. | | | up to 10 μM | up to 10 μM |
| PT-43. | 3-(4-hydroxyphenyl)-7-[(3-phenyl(1,2,4- | 0.47 | μM | No | No |
| | oxadiazol-5-yl))methoxy]chromen-4-one; | | | inhibition | inhibition |
| | | | | up to 30 μM | up to 30 μM |
| PT-44. | 3-(4-hydroxyphenyl)-7-({3-[4- | 0.27 | μM | No | No |
| | chlorophenyl](1,2,4-oxadiazol-5- | | | inhibition | inhibition |
| | yl)}methoxy)chromen-4-one; | | | up to 30 μM | up to 30 μM |
| PT-45. | 3-(4-hydroxyphenyl)-7-({5-[3- | 0.098 | μM | No | 7% |
| | (trifluoromethyl)phenyl]isoxazol-3- | | | inhibition | inhibition |
| | yl}methoxy)chromen-4-one; | | | up to 10 μM | at 10 μM |
| PT-46. | 7-{[5-(trifluoromethyl)(3-pyridyl)]methoxy}-3- | 10% | | | |
| | (4-{[6-(trifluoromethyl)(3- | inhibition | | | |
| | pyridyl)]methoxy}phenyl)chromen-4-one; | at 1 μM | | | |
| PT-47. | methyl 2-{[3-(4-hydroxyphenyl)-4- | 0.005 | μM | No | 34% |
| | oxochromen-7-yloxy]methyl}-1,3-oxazole-5- | | | inhibition | inhibition |
| | carboxylate; | | | up to 10 μM | at 10 μM |
| PT-48. | 7-{[5-(4-fluorophenyl)(1,2,4-oxadiazol-3- | 0.14 | μM | | |
| | yl)]methoxy}-3-{4-[(methylsulfonyl)amino]- | | | | |
| | phenyl}chromen-4-one; | | | | |
| PT-49. | 2-{[3-(4-hydroxyphenyl)-4-oxochromen-7- | 0.016 | μM | No | |
| | yloxy]methyl}-1,3-oxazole-5-carboxylic acid; | | | inhibition | |
| | | | | up to 10 μM | |
| PT-50. | methyl 3-({3-[4-((1Z)-1-amino-2-methoxy-2- | 47% | | | |
| | azavinyl)phenyl]-4-oxochromen-7- | inhibition | | | |
| | yloxy}methyl)benzoate; | at 1 μM | | | |
| PT-51. | 7-{2-[4-(4-chlorophenyl)pyrazolyl]ethoxy}-3- | 0.11 | μM | | |
| | (4-hydroxyphenyl)chromen-4-one; | | | | |
| PT-52. | 3-(4-hydroxyphenyl)-7-[(6-pyrazolyl(3- | 0.01 | μM | No | |
| | pyridyl))methoxy]chromen-4-one; | | | inhibition | |
| | | | | up to 10 μM | |
| PT-53. | 7-[(2R)-2-hydroxy-3-({[3- | 0.016 | μM | No | 19% |
| | (trifluoromethyl)phenyl]methyl}amino)propoxy]- | | | inhibition | inhibition |
| | 3-(4-hydroxyphenyl)chromen-4-one; | | | up to 10 μM | at 10 μM |
| PT-54. | 3-(4-hydroxyphenyl)-7-[({[3- | 0.005 | μM | No | |
| | (trifluoromethyl)phenyl]methyl}amino)methoxy] | | | inhibition | |
| | chromen-4-one; | | | up to 10 μM | |
| PT-55. | 7-((2R)-3-{[(3,5- | 0.008 | μM | No | 14% |
| | difluorophenyl)methyl]amino}-2- | | | inhibition | inhibition |
| | hydroxypropoxy)-3-(4- | | | up to 10 μM | at 10 μM |
| | hydroxyphenyl)chromen-4-one; | | | | |
| PT-56. | 7-(3-{[(1R)-1-(4-fluorophenyl)ethyl]amino}-2- | 0.008 | μM | 36% | 29% |
| | oxopropoxy)-3-(4-hydroxyphenyl)chromen-4- | | | inhibition | inhibition |
| | one; | | | up to 10 μM | at 10 μM |
| PT-57. | 3-(4-hydroxyphenyl)-7-(3- | 0.02 | μM | No | 27% |
| | phenylpropoxy)chromen-4-one; | | | inhibition | inhibition |
| | | | | up to 10 μM | at 10 μM |
| PT-58. | 7-{[5-(3-fluorophenyl)(1,3,4-oxadiazol-2- | 0.011 | μM | No | 25% |
| | yl)]methoxy}-3-(4-hydroxyphenyl)chromen-4- | | | inhibition | inhibition |
| | one; | | | up to 10 μM | at 10 μM |
| PT-59. | 3-(4-hydroxyphenyl)-7-{[3- | 0.67 | μM | No | 24% |
| | (trifluoromethyl)phenyl]ethoxy}chromen-4-one; | | | inhibition | inhibition |
| | | | | up to 10 μM | at 10 μM |
| PT-60. | 3-(4-hydroxyphenyl)-7-({5-[3- | 0.042 | μM | No | 13% |
| | (trifluoromethyl)phenyl](1,3,4-oxadiazol-2- | | | inhibition | inhibition |
| | yl)}methoxy)chromen-4-one; | | | up to 10 μM | at 10 μM |
| PT-61. | 3-(4-hydroxyphenyl)-7-[(2-phenyl(1,3-oxazol- | 0.096 | μM | No | 17% |
| | 5-yl))methoxy]chromen-4-one; | | | inhibition | inhibition |
| | | | | up to 10 μM | at 10 μM |
| PT-62. | 7-({5-[3,5-bis(trifluoromethyl)phenyl]isoxazol- | 0.072 | μM | No | no |
| | 3-yl}methoxy)-3-(4-hydroxyphenyl)chromen-4- | | | inhibition | inhibition |
| | one; | | | up to 10 μM | at 10 μM |
| PT-63. | 3-(4-hydroxyphenyl)-7-({5-[3- | 0.098 | μM | No | 7% |
| | (trifluoromethyl)phenyl]isoxazol-3- | | | inhibition | inhibition |
| | yl}methoxy)chromen-4-one; | | | up to 10 μM | at 10 μM |
| PT-64. | 7-{[5-(2-chlorophenyl)(1,3,4-thiadiazol-2- | 43% | | No | 8% |
| | yl)]methoxy}-3-(4-hydroxyphenyl)chromen-4- | inhibition | | inhibition | inhibition |
| | one; | at 1 μM | | up to 10 μM | at 10 μM |
| PT-65. | 4-[7-({4-methyl-2-[4- | 30% | | No | 25% |
| | (trifluoromethyl)phenyl](1,3-thiazol-5- | inhibition | | inhibition | inhibition |
| | yl)}methoxy)-4-oxochromen-3- | at 1 μM | | up to 10 μM | at 10 μM |
| | yl]benzenecarbonitrile; | | | | |
| PT-66. | 3-{4-[(methylsulfonyl)amino]phenyl}-7-({4- | 48% | | No | 25% |
| | methyl-2-[4-(trifluoromethyl)phenyl](1,3- | inhibition | | inhibition | inhibition |
| | thiazol-5-yl)}methoxy)chromen-4-one; | at 1 μM | | up to 10 μM | at 10 μM |
| PT-67. | 3-(6-methoxy(3-pyridyl))-7-({4-methyl-2-[4- | 25% | | No | 16% |
| | (trifluoromethyl)phenyl](1,3-thiazol-5- | inhibition | | inhibition | inhibition |
| | yl)}methoxy)chromen-4-one; | at 1 μM | | up to 10 μM | at 10 μM |
| PT-68. | 4-[7-({5-[5-fluoro-3- | 33% | | No | 14% |
| | (trifluoromethyl)phenyl](1,3,4-oxadiazol-2- | inhibition | | inhibition | inhibition |
| | yl)}methoxy)-4-oxochromen-3- | at 1 μM | | up to 10 μM | at 10 μM |
| | yl]benzenecarbonitrile; | | | | |
| PT-69. | 4-[4-oxo-7-({3-[3- | 0.18 | μM | No | |
| | (trifluoromethyl)phenyl]isoxazol-5- | | | inhibition | |
| | yl}methoxy)chromen-3-yl]benzenecarbonitrile; | | | up to 10 μM | |
| PT-70. | 7-({5-[3-fluoro-5- | 20% | | No | 11% |
| | (trifluoromethyl)phenyl](1,2,4-oxadiazol-3- | inhibition | | inhibition | inhibition |
| | yl)}methoxy)-3-{4- | at 1 μM | | up to 10 μM | at 10 μM |
| | [(methylsulfonyl)amino]phenyl}chromen-4-one; | | | | |
| PT-71. | 7-({5-[3-fluoro-5- | 8% | | No | 11% |
| | (trifluoromethyl)phenyl](1,2,4-oxadiazol-3- | inhibition | | inhibition | inhibition |
| | yl)}methoxy)-3-[4- | at 1 μM | | up to 10 μM | at 10 μM |
| | (methylsulfonyl)phenyl]chromen-4-one; | | | | |
| PT-72. | 4-[7-({5-[3-fluoro-5- | 14% | | No | No |
| | (trifluoromethyl)phenyl](1,2,4-oxadiazol-3- | inhibition | | inhibition | inhibition |
| | yl)}methoxy)-4-oxochromen-3-yl]benzamide; | at 1 μM | | up to 10 μM | at 10 μM |
| PT-73. | 3-(3-acetylphenyl)-7-({5-[3-fluoro-5- | 18% | | No | 10% |
| | (trifluoromethyl)phenyl](1,2,4-oxadiazol-3- | inhibition | | inhibition | inhibition |
| | yl)}methoxy)chromen-4-one; | at 1 μM | | up to 10 μM | at 10 μM |
| PT-74. | 7-({5-[3-fluoro-5- | 0.005 | μM | No | 22% |
| | (trifluoromethyl)phenyl](1,3,4-oxadiazol-2- | | | inhibition | inhibition |
| | yl)}methoxy)-3-(4-hydroxyphenyl)chromen-4- | | | up to 10 μM | at 10 μM |
| | one; | | | | |
| PT-75. | 7-({5-[3-fluoro-5- | 14% | | No | No |
| | (trifluoromethyl)phenyl](1,2,4-oxadiazol-3- | inhibition | | inhibition | inhibition |
| | yl)}methoxy)-3-(5-hydropyrazol-4-yl)chromen- | at 1 μM | | up to 10 μM | at 10 μM |
| | 4-one; | | | | |
| PT-76. | ethyl 3-[7-({3-[3-fluoro-5- | 0.063 | μM | No | 21% |
| | (trifluoromethyl)phenyl](1,2,4-oxadiazol-5- | | | inhibition | inhibition |
| | yl)}ethoxy)-4-oxochromen-3-yl]benzoate; | | | up to 10 μM | at 10 μM |
| PT-77. | 3-(4-hydroxyphenyl)-7-({2-[4- | 0.122 | μM | | |
| | (trifluoromethyl)phenyl](1,3-thiazol-5- | | | | |
| | yl)}methoxy)chromen-4-one; | | | | |
| PT-78. | 7-[2-(3-fluorophenyl)-2-oxoethoxy]-3-(4- | 0.139 | μM | | |
| | hydroxyphenyl)chromen-4-one; | | | | |
| PT-79. | 7-({5-[3-fluoro-5- | 0.048 | μM | No | 18% |
| | (trifluoromethyl)phenyl](1,2,4-oxadiazol-3- | | | inhibition | inhibition |
| | yl)}ethoxy)-3-(4-hydroxyphenyl)chromen-4- | | | up to 10 μM | at 10 μM |
| | one; | | | | |
| PT-80. | 7-{[5-(2-chlorophenyl)(1,3,4-oxadiazol-2- | 0.004 | μM | No | No |
| | yl)]methoxy}-3-(4-hydroxyphenyl)chromen-4- | | | inhibition | inhibition |
| | one; | | | up to 10 μM | at 10 μM |
| PT-81. | 7-{[5-(4-fluorophenyl)(1,3,4-oxadiazol-2- | 0.0004 | μM | No | 12% |
| | yl)]methoxy}-3-(4-hydroxyphenyl)chromen-4- | | | inhibition | inhibition |
| | one; | | | up to 10 μM | at 10 μM |
| PT-82. | 3-(4-hydroxyphenyl)-7-(4- | 0.005 | μM | No | 15% |
| | pyridylmethoxy)chromen-4-one; | | | inhibition | inhibition |
| | | | | up to 10 μM | at 10 μM |
| PT-83. | 3-{4-[(methylsulfonyl)amino]phenyl}-7-({2-[4- | 0.025 | μM | | |
| | (trifluoromethyl)phenyl](1,3-thiazol-5- | | | | |
| | yl)}methoxy)chromen-4-one; | | | | |
| PT-84. | 2-[3-(4-hydroxyphenyl)-4-oxochromen-7- | 0.015 | μM | No | 20% |
| | yloxy]-N-[2-(trifluoromethyl)phenyl]- | | | inhibition | inhibition |
| | acetamide; | | | up to 10 μM | at 10 μM |
| PT-85. | 3-(4-hydroxyphenyl)-7-{2-oxo-2-[2- | 0.07 | μM | No | No |
| | (trifluoromethyl)phenyl]ethoxy}chromen-4-one; | | | inhibition | inhibition |
| | | | | up to 10 μM | at 10 μM |
| PT-86. | 3-(1H-indazol-5-yl)-7-({5-[5-fluoro-3- | 68% | | | |
| | (trifluoromethyl)phenyl](1,2,4-oxadiazol-3- | inhibition | | | |
| | yl)}methoxy)chromen-4-one; | at 1 μM | | | |
| PT-87. | 3-(4-hydroxyphenyl)-7-(2- | 0.040% | | No | 21% |
| | phenylethoxy)chromen-4-one; | inhibition | | inhibition | inhibition |
| | | at 1 μM | | up to 10 μM | at 10 μM |
| PT-88. | 2-[3-(4-hydroxyphenyl)-4-oxochromen-7- | 0.023 | μM | 6.2 μM | 35% |
| | yloxy]ethanenitrile; | | | | inhibition |
| | | | | | at 10 μM |
| PT-89. | 7-[2-(4-chlorophenoxy)ethoxy]-3-(4- | 0.022 | μM | 34% | 32% |
| | hydroxyphenyl)chromen-4-one; | | | inhibition | inhibition |
| | | | | up to 10 μM | at 10 μM |
| PT-90. | N-[(1R)-1-(4-fluorophenyl)ethyl]-2-[3-(4- | 0.006 | μM | No | 12% |
| | hydroxyphenyl)-4-oxochromen-7- | | | inhibition | inhibition |
| | yloxy]acetamide; | | | up to 10 μM | at 10 μM |
| PT-91. | 3-(4-hydroxyphenyl)-7-(2- | 0.007 | μM | No | 11% |
| | pyridylmethoxy)chromen-4-one; | | | inhibition | inhibition |
| | | | | up to 10 μM | at 10 μM |
| PT-92. | 2-fluoro-5-[7-({5-[5-fluoro-3- | 24% | | | |
| | (trifluoromethyl)phenyl](1,2,4-oxadiazol-3- | inhibition | | | |
| | yl)}methoxy)-4-oxochromen-3- | at 1 μM | | | |
| | yl]benzenecarbonitrile; | | | | |
| PT-93. | 7-(2-pyridylmethoxy)-3-[4-(2- | 0.017 | μM | | |
| | pyridylmethoxy)phenyl]chromen-4-one; | | | | |
| PT-94. | 3-(4-hydroxyphenyl)-7-{[5-(trifluoromethyl)(3- | 0.02 | μM | No | No |
| | pyridyl)]methoxy}chromen-4-one; | | | inhibition | inhibition |
| | | | | up to 10 μM | at 10 μM |
| PT-95. | 7-{[5-(4-chlorophenyl)isoxazol-3-yl]methoxy}- | 57% | | | |
| | 3-(4-hydroxyphenyl)chromen-4-one; | inhibition | | | |
| | | at 1 μM | | | |
| PT-96. | 7-{[5-(3,4-dichlorophenyl)isoxazol-3- | 47% | | | |
| | yl]methoxy}-3-(4-hydroxyphenyl)chromen-4- | inhibition | | | |
| | one; | at 1 μM | | | |
| PT-97. | 7-{[5-(4-chlorophenyl)isoxazol-3-yl]methoxy}- | 57% | | | |
| | 3-(4-hydroxyphenyl)chromen-4-one; | inhibition | | | |
| | | at 1 μM | | | |
| PT-98. | 7-[(2R)-2-hydroxy-3-({[3- | 0.016 | μM | No | 19% |
| | (trifluoromethyl)phenyl]methyl}amino)propoxy]- | | | inhibition | inhibition |
| | 3-(4-hydroxyphenyl)chromen-4-one; | | | up to 10 μM | at 10 μM |
| PT-99. | 3-(4-hydroxyphenyl)-7-[2-({[3- | 0.005 | μM | No | |
| | (trifluoromethyl)phenyl]methyl}amino)ethoxy]chromen- | | | inhibition | |
| | 4-one; | | | up to 10 μM | |
| PT-100. | 7-((2R)-3-{[(3,5- | 0.008 | μM | No | 14% |
| | difluorophenyl)methyl]amino}-2- | | | inhibition | inhibition |
| | hydroxypropoxy)-3-(4- | | | up to 10 μM | at 10 μM |
| | hydroxyphenyl)chromen-4-one; | | | | |
| PT-101. | methyl 2-{[3-(4-hydroxyphenyl)-4- | 0.005 | μM | No | 34% |
| | oxochromen-7-yloxy]methyl}-1,3-oxazole-4- | | | inhibition | inhibition |
| | carboxylate; | | | up to 10 μM | at 10 μM |
| PT-102. | 2-{[3-(4-hydroxyphenyl)-4-oxochromen-7- | 0.016 | μM | No | |
| | yloxy]methyl}-1,3-oxazole-4-carboxylic acid; | | | inhibition | |
| | | | | up to 10 μM | |
| PT-103. | N-[(1S)-1-(4-fluorophenyl)ethyl]-2-[3-(4- | 0.008 | μM | 36% | 29% |
| | hydroxyphenyl)-4-oxochromen-7- | | | inhibition | inhibition |
| | yloxy]acetamide; | | | up to 10 μM | at 10 μM |
| PT-104. | 7-{[5-(4-fluorophenyl)(1,2,4-oxadiazol-3- | 0.016 | μM | No | No |
| | yl)]methoxy}-3-(4-hydroxyphenyl)chromen-4- | | | inhibition | inhibition |
| | one; | | | up to 10 μM | at 10 μM |
| PT-105. | 7-{[5-(4-fluorophenyl)(1,2,4-oxadiazol-3- | 0.14 | μM | | |
| | yl)]methoxy}-3-{4-[(methylsulfonyl)- | | | | |
| | amino]phenyl}chromen-4-one; | | | | |
| PT-106. | 7-{3-[4-(4-chlorophenyl)pyrazolyl]propoxy}-3- | 0.11 | μM | | |
| | (4-hydroxyphenyl)chromen-4-one; | | | | |
| PT-107. | 3-(4-hydroxyphenyl)-7-(3- | 0.02 | μM | No | 27% |
| | phenylpropoxy)chromen-4-one; | | | inhibition | inhibition |
| | | | | up to 10 μM | at 10 μM |
| PT-108. | 3-(4-hydroxyphenyl)-7-[(6-pyrazolyl(3- | 0.010 | μM | No | |
| | pyridyl))methoxy]chromen-4-one; | | | inhibition | |
| | | | | up to 10 μM | |
| PT-109. | 7-((2R)-2-hydroxy-3-phenylpropoxy)-3-(4- | 0.014 | μM | 26% | 26% |
| | hydroxyphenyl)chromen-4-one; | | | inhibition | inhibition |
| | | | | up to 10 μM | at 10 μM |
| PT-110. | 3-(4-hydroxyphenyl)-7-[(5-(3-pyridyl)(1,3,4- | 0.007 | μM | No | No |
| | oxadiazol-2-yl))methoxy]chromen-4-one; | | | inhibition | inhibition |
| | | | | up to 10 μM | at 10 μM |
| PT-111. | 3-[(2-hydroxy-3-{4- | 0.003 | μM | No | 30% |
| | [(methylsulfonyl)amino]phenyl}-4- | | | inhibition | inhibition |
| | oxochromen-7-yloxy)methyl]-benzoic acid; | | | up to 10 μM | at 10 μM |
| PT-112. | 7-{[5-(4-fluorophenyl)(1,3,4-oxadiazol-2- | 0.005 | μM | No | No |
| | yl)]ethoxy}-3-(4-hydroxyphenyl)chromen-4- | | | inhibition | inhibition |
| | one; | | | up to 10 μM | at 10 μM |
| PT-113. | 3-(4-hydroxyphenyl)-7-[(5-(3-pyridyl)(1,3,4- | 0.017 | μM | No | 30% |
| | oxadiazol-2-yl))ethoxy]chromen-4-one; | | | inhibition | inhibition |
| | | | | up to 10 μM | at 10 μM |
| PT-114. | 3-(4-hydroxyphenyl)-7-[(3-(3-pyridyl)(1,2,4- | 0.032 | μM | No | No |
| | oxadiazol-5-yl))methoxy]chromen-4-one; | | | inhibition | inhibition |
| | | | | up to 10 μM | at 10 μM |
| PT-115. | 3-(4-hydroxyphenyl)-7-({3-[3- | 0.038 | μM | No | No |
| | (trifluoromethyl)phenyl](1,2,4-oxadiazol-5- | | | inhibition | inhibition |
| | yl)}methoxy)chromen-4-one; | | | up to 10 μM | at 10 μM |
| PT-116. | 3-(4-hydroxyphenyl)-7-[(5-(3-pyridyl)(1,3,4- | 0.015 | μM | No | 33% |
| | oxadiazol-2-yl))ethoxy]chromen-4-one; | | | inhibition | inhibition |
| | | | | up to 10 μM | at 10 μM |
| PT-117. | 3-(4-hydroxyphenyl)-7-[(5-(4-pyridyl)(1,2,4- | 0.098 | μM | No | No |
| | oxadiazol-3-yl))ethoxy]chromen-4-one; | | | inhibition | inhibition |
| | | | | up to 10 μM | at 10 μM |
| PT-118. | (2-{[3-(4-hydroxyphenyl)-4-oxochromen-7- | 0.023 | μM | No | No |
| | yloxy]methyl}(1,3-oxazol-4-yl))-N- | | | inhibition | inhibition |
| | methylcarboxamide; | | | up to 10 μM | at 10 μM |
| PT-119. | 4-{[3-(4-hydroxyphenyl)-4-oxochromen-7- | 0.068 | μM | No | No |
| | yloxy]methyl}-7-methoxychromen-2-one; | | | inhibition | inhibition |
| | | | | up to 10 μM | at 10 μM |
| PT-120. | 7-{[5-(4-fluorophenyl)(1,3,4-oxadiazol-2- | 0.276 | μM | | |
| | yl)]methoxy}-3-{4-[(methylsulfonyl)amino]- | | | | |
| | phenyl}chromen-4-one; | | | | |
| PT-121. | 7-{[5-(3-aminophenyl)(1,3,4-oxadiazol-2- | 0.011 | μM | No | No |
| | yl)]methoxy}-3-(4-hydroxyphenyl)chromen-4- | | | inhibition | inhibition |
| | one; | | | up to 10 μM | at 10 μM |
| PT-122. | ethyl 1-{2-[3-(4-hydroxyphenyl)-4- | 0.012 | μM | No | No |
| | oxochromen-7-yloxy]ethyl}pyrazole-4- | | | inhibition | inhibition |
| | carboxylate; | | | up to 10 μM | at 10 μM |
| PT-123. | 7-{2-[4-(3-chlorophenyl)piperazinyl]ethoxy}-3- | 0.011 | μM | No | |
| | (4-hydroxyphenyl)chromen-4-one; | | | inhibition | |
| | | | | up to 10 μM | |
| PT-124. | 3-(4-hydroxyphenyl)-7-(2-{4-[3- | 0.018 | μM | No | 21% |
| | (trifluoromethyl)phenyl]piperazinyl}ethoxy)chromen- | | | inhibition | inhibition |
| | 4-one; | | | up to 10 μM | at 10 μM |

### EXAMPLE 31

### Rodent Models of Anxiety

Anxiolytic properties of Compound A (3-[(3-{4-[(methylsulfonyl)amino]phenyl}-4-oxochromen-7-yloxy)methyl]benzoic acid, a compound of Formula I) were tested in four rodent model systems exhibiting anxiety-like behavior: congenitally anxious Fawn-Hooded (FH) rats, repeated alcohol withdrawal-induced anxiety (Overstreet et al., Alcohol Clin Exp Res (2002) 26:1259-1269), restraint-induced anxiety (Breese et al., Neuropsychopharmacology (2004) 29:470-482) and drug-induced anxiety produced by DMCM, a benzodiazepine inverse receptor agonist (Overstreet et al., Pharmacol Biochem Behav (2003) 75:619-625; Stephens et al., 1983).

FH rats (300 g) were about 70 days of age and Sprague-Dawley (SD) rats (Charles-River, Raleigh, N.C.) were about 50 days of age. Rats were housed in a standard animal environment with temperatures at 22° C. and humidity at 50%. The light:dark cycle was 0700-1900, with lights on at 0700.

Social Interaction Test: Decreased social interaction has been validated repeatedly as an index of anxiety-like behavior (See File and Seth, Eur J. Pharmacol. (2003) 463(1-3):35-53.) following anxiogenic stimuli such as bright lights, exposure to cat odor (File, J Neurosci Meth (1980) 2: 219-238); File and Hyde, Br J Pharmacol (1978) 62:19-24), after administration of anxiogenic drugs (e.g., Bhattacharya et al., J Psychopharmacol (1997) 11:219-224; File and Lister, Neuropharmacology (1984) 23:793-796; Guy and Gardner, Neuropsychobiology (1985) 13:194-200) or following withdrawal from drugs of abuse, including alcohol (Andrews et al., Psychopharmacology (1997) 130:228-234; Costall et al., Pharmacol Biochem Behav (1990) 36:339-344; File et al., Psychopharmacology (1989) 98: 262-264; Irvine et al., Psychopharmacology (2001) 153: 315-320; Kampov-Polevoy et al., Alcohol Clin Exp Res (2000) 24: 278-284). Conversely, social interaction is increased by prior exposure to the test arena (File 1980; File and Hyde, 1978) or the administration of anxiolytic drugs, preferably at doses that do not effect locomotor activity (File, 1980; Lightowler et al., Pharmacol Biochem Behav (1994) 49:281-285).

Experienced observers blinded to the experimental conditions carried out the social interaction test in a square open field (60 cm by 60 cm, with 16 15×15 cm squares marked out on the floor). Pairs of rats receiving the same treatment were placed in the arena and the onset of social interactions recorded. This procedure has been validated by previous studies (Overstreet et al. (2002); Overstreet et al. (2003)). Rats were unfamiliar with the open field and lighting conditions were low in order to generate an intermediate level of anxiety-related behavior. Rat pairs matched according to weight and treatment were placed simultaneously in the open field. During the 5-min session, line crosses (by two forepaws) and time spent in social interaction (grooming, sniffing, following) were scored individually for each rat (Kampov-Polevoy et al., 2000; Overstreet et al., 2002, 2003).

Statistical Analyses: Data were analyzed by statistical analyses appropriate for the research designs. FH studies were analyzed by unpaired t tests for the two groups. Alcohol withdrawal-, restraint- and chemically-induced anxiety were analyzed by one-way ANOVA, using Tukey-Kramer tests to identify significant results.

Fawn Hooded (FH) rat model: FH rats were randomly assigned to one of two treatment groups, each containing 7 rats. One group received 1 ml/kg of 0.5% carboxymethylcellulose (CMC) (vehicle). The other group received an i.p. injection of 15 mg/kg Compound A in vehicle. Thirty minutes (min) after the injection, rats were placed in an open field arena for recording social interaction and line crosses in a 5-min session.

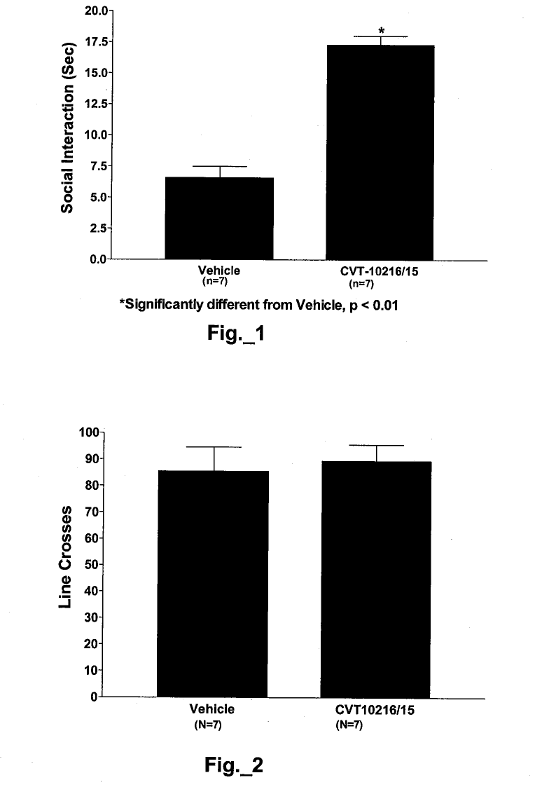

FH rats are characterized by innate increased anxiety compared to other rat strains. FH rats treated with vehicle exhibited very low social interaction (FIG. 1). However, FH rats treated with Compound A exhibited less anxiety, indicated by a 2-fold increase in social interaction (FIG. 1; t=9.03, p<0.001). In contrast, CVT-10216 did not affect locomotor line crossing (FIG. 2; t=0.35, NS).

Ethanol Exposure Studies: Individually housed Sprague-Dawley (SD) rats were given a complete nutritious liquid diet (Knapp et al., 2004; Overstreet et al., 2002) after 5 days to adapt to local conditions. Three days later, most of rats received a 4.5% ethanol liquid diet and others remained on the control liquid diet without ethanol. Rats consumed ethanol for 15 days in three cycles of 5 days each separated by a 2-day withdrawal period between cycles. This design enabled us to test two treatment regimens with Compound A. Acute effects of Compound A (3.75 and 15 mg/kg) were determined 4.5 hr after the 3rd ethanol cycle to allow alcohol washout. The social interaction test was carried out 30 min later. Prophylactic effects were determined 5 hr after the 3^{rd} cycle when the rats had received Compound A (1.875-15 mg/kg) after the 1^{st} and 2^{nd} cycles, but not the 3^{rd} cycle. Thus, prophylactically treated rats had not received Compound A for 5 days before social interaction testing 5 hr after ethanol was removed in the third cycle.

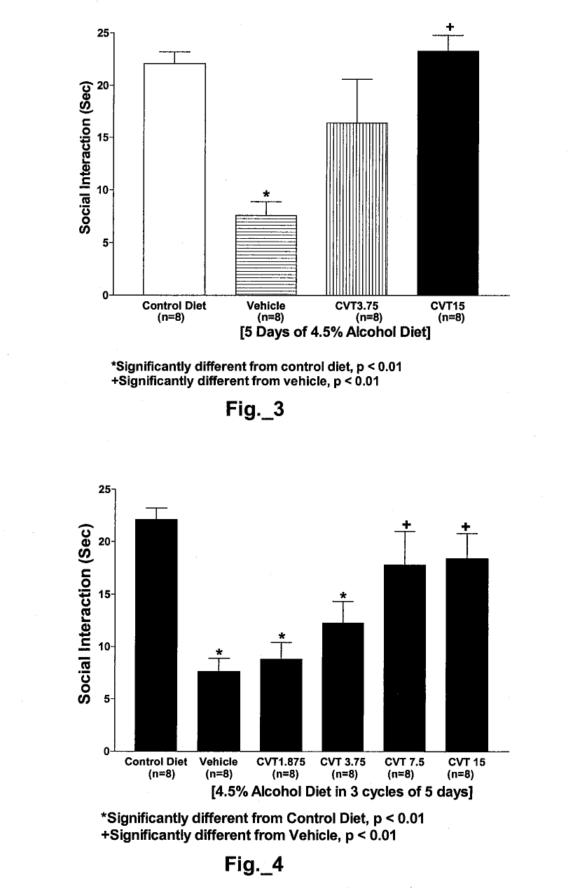

Repeated alcohol-withdrawal caused a reduction in social interaction (FIG. 3; data represent the mean±s.e.m. for 8 rats). Compound A injected 30 min before testing increased the time of social interaction produced by alcohol withdrawal, with higher doses completely normalizing the behavior. The one-way ANOVA was statistically significant (F[3,43]=11.63, p<0.001), confirming the anxiolytic effect of Compound A. No significant differences in locomotor activity were detected (F[3,43]=2.16, NS; data not shown).

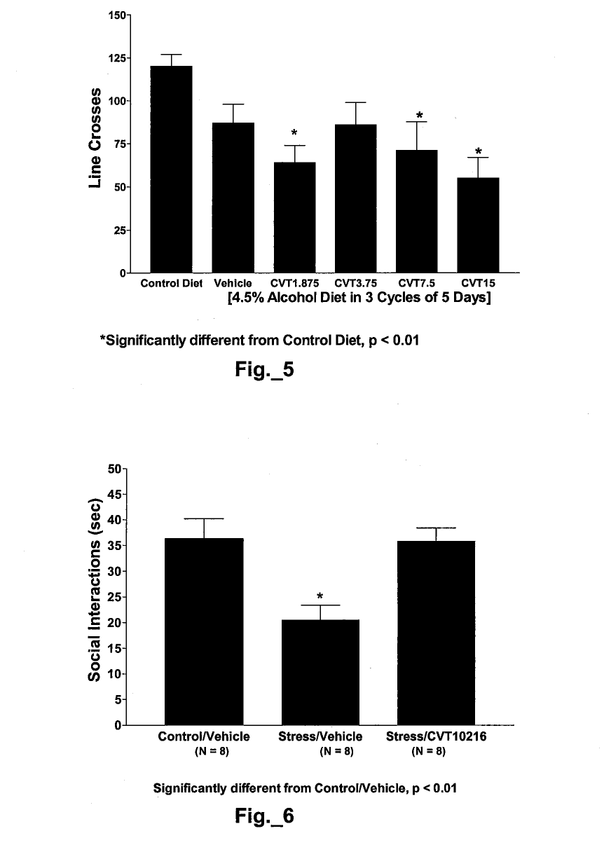

Importantly, Compound A (3.75-15 mg/kg) increased social interaction behavior prophylactically when given in a regimen that included administration of Compound A during the 1^{st} and 2^{nd} withdrawals and no administration of Compound A after the 3^{rd} cycle (FIG. 4; data represent the mean±s.e.m. for 8 rats). Thus, testing was done 5 days after the last dose of Compound A and significant group differences were confirmed (F[5,57]=11.02, p<0.001), suggesting that Compound A interfered with anxiogenic CNS changes that develop as a consequence of ethanol withdrawals. There was a barely significant effect of Compound A on locomotor activity when compared to vehicle treated rats withdrawn from the alcohol liquid diet (STATS) (FIG. 5).

Restraint-Induced Stress Study: SD rats were injected with either CMC vehicle or 15 mg/kg Compound A in vehicle and subjected to restraint stress in decapicones for one hour. After a 30-min recovery period, the rats were exposed to the open field arena for 5 mm and social interaction and line crosses recorded.

Restraint stress for 60 min resulted in a significant reduction in social interaction; pretreatment with Compound A prevented stress-induced anxiety-like behavior. Tukey-Kramer tests confirmed differences between control and stressed rats as well as between vehicle- and Compound A-treated rats (FIG. 6; data represent the mean±s.e.m. for 8 rats; F[2,19]=6.82, p<0.01). There were no significant (F[2,19]=2.12, NS) differences in locomotor activity (data not shown).

Chemically Induced Anxiogenic Effects: Sprague-Dawley Rats each received one of four i.p. treatments: CMC vehicle or Compound A (3.75, 7.5 or 15 mg/kg) in vehicle. Fifteen min later the rats received a second injection of 0.5 mg/kg DMCM (methyl 6,7-demethoxy-4-ethyl-β-carboline-3-carboxylate) or vehicle (acidified saline). The rats were placed in the open field arena 15 min after the second injection for recording social interaction and locomotor activity for 5-min. Approximately one week later the rats were treated with Compound A (3.75, 7.5 or 15 mg/kg). The 5-HT_{2C} agonist mCPP (m-chlorophenylpiperazine; 0.5 mg/kg) or vehicle (saline) was given 15 min later. After another 15 min period, the social interaction and locomotor activity for five min were recorded.

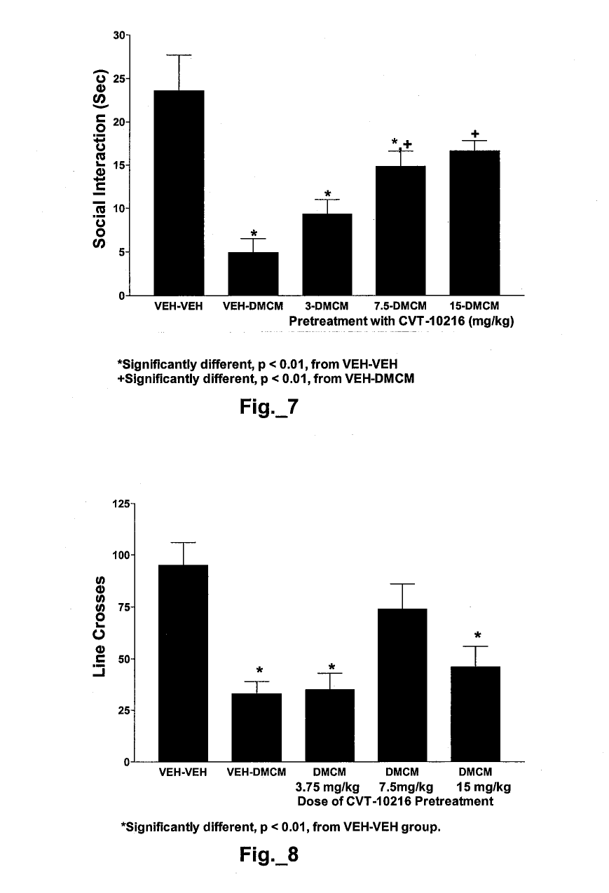

DMCM, a benzodiazepine inverse agonist, substantially reduced social interaction (FIG. 7). However, these anxiogenic effects were prevented by Compound A in a dose-dependent manner, as shown by a significant one-way ANOVA (F[4,35]=9.57, p<0.0001) and subsequent Tukey's tests. On the other hand, reduced locomotor activity caused by DMCM was not affected by Compound A, except at 7.5 mg/kg (FIG. 8). There were significant group effects (F[4,35]=7.74, p<0.0001) and Tukey's test confirmed that DMCM reduced locomotor activity when compared to controls.

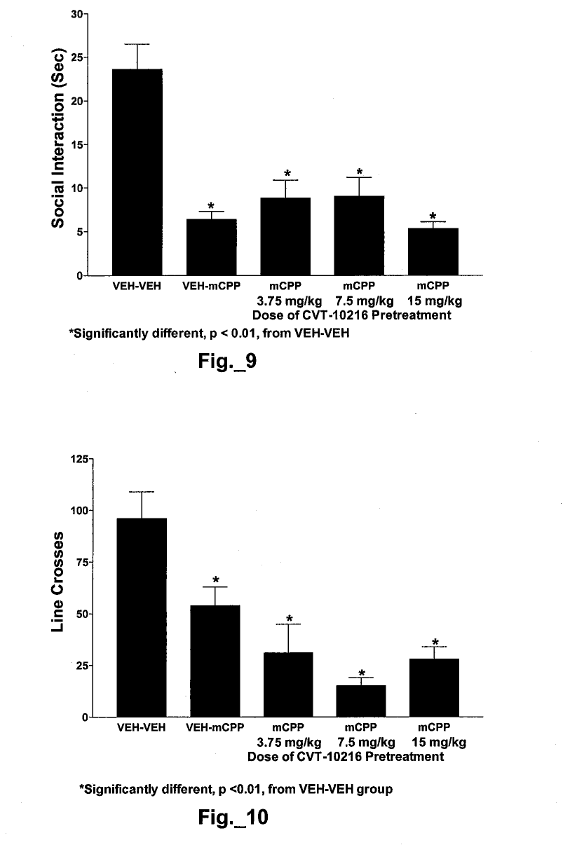

Substantial anxiety-like behavior is produced by mCPP, a 5-HT_{2C} antagonist, as indicated by a low social interaction score (FIG. 9). Compound A did not prevent mCPP-induced anxiogenesis. All groups receiving mCPP were similar to each other and very different from the controls (FIG. 9; F[4.35]=21.71, p<0.0001). There was no effect of mCPP on locomotor activity when compared to vehicle-treated rats receiving mCPP (FIG. 10). But, there were significant group differences (F[4,35]=8.92, p<0.0001) and Tukey's tests confirmed that each mCPP reduced locomotor activity when compared to controls.

These results support a finding that Compound A, highly selective reversible ALDH-2 inhibitor, or other compounds of Formula I, exhibit unequivocal anxiolytic properties. Compound A has shown these anxiolytic properties in four different rat models of anxiety-like behavior. The social interaction test described above measured anxiolytic properties of Compound A against endogenous anxiety-like behavior in naïve FH rats, alcohol withdrawal-induced anxiety, stress-induced anxiety and drug-induced anxiety. Locomotor activity was also assayed as a control for non-specific effects on neural function. Compound A was found to have virtually no effect on locomotor activity when assayed in all of the models tested. Indeed, changes in locomotor activity were rarely observed when Compound A exhibited clear dose-dependent anxiolytic properties. In summary, Compound A exhibits anxiolytic properties in several well established rodent model systems.

### EXAMPLE 32

### Reduction of Anxiety Increase in Social Interaction

Rats (n=6) were given vehicle or Compound A, 3-[(3-{4-[(methylsulfonyl)amino]phenyl}-4-oxochromen-7-yloxy)methyl]benzoic acid, (15 mg/kg) 30 min before the social interaction and 30-60 min before lights went out. One noninjected rat was included in the control group for water intake. Significant results: Compound A increased social interaction (anxiolytic effect) but not activity. Compound A also reduced alcohol intake from 2-6 hr, but did not affect food or water intake.

| | Social | | Line | | Center | | Food | |
|---|---|---|---|---|---|---|---|---|
| | Interaction | | Crosses | | Entries | | Intake | |
| | Comp. A | VEH | Comp. A | VEH | Comp. A | VEH | Comp. A | VEH |
| | 13 | 7 | 135 | 98 | 28 | 17 | 19 | 26 |
| | 16 | 8 | 125 | 103 | 25 | 13 | 18 | 22 |
| | 14 | 9 | 84 | 106 | 18 | 26 | 19 | 22 |
| | 11 | 4 | 113 | 132 | 13 | 28 | 21 | 18 |
| | 12 | 2 | 104 | 84 | 26 | 10 | 21 | 24 |
| | 14 | 6 | 90 | 85 | 6 | 13 | 20 | 23 |
| Mean | 13.3 | 6 | 109 | 101 | 19.3 | 17.8 | 19.7 | 22.5 |
| s.e.m. | 0.7 | 1.1 | 8 | 7 | 3.5 | 3 | 0.5 | 1.1 |

| | | | | |
|---|---|---|---|---|
| t value | t = 5.72, | t = 0.66, NS | t = 0.32, NS | t = 2.37, |
| | p < 0.001 | | | p > 0.05 |

| | 2 hr | | 4 hr | | 6 hr | | 24 hr | |
|---|---|---|---|---|---|---|---|---|
| | Alcohol | | Alcohol | | Alcohol | | Alcohol | |
| | Intake | | Intake | | Intake | | Intake | |
| | Comp. A | VEH | Comp. A | VEH | Comp. A | VEH | Comp. A | VEH |
| | 0 | 0.38 | 0.4 | 0.76 | 1.2 | 1.52 | 4 | 3.2 |
| | 0.38 | 1.44 | 0.76 | 2.7 | 1.23 | 3.23 | 4.94 | 8.1 |
| | 0.38 | 0.19 | 0.95 | 0.38 | 1.71 | 0.76 | 5.32 | 1.9 |
| | 0.38 | 0.19 | 0.38 | 0.38 | 0.95 | 0.95 | 3.23 | 3.04 |
| | 0.19 | 1.14 | 0.57 | 2.09 | 1 | 3.61 | 3.6 | 6.82 |
| | 0 | 1.33 | 0.88 | 2.66 | 1.72 | 3.61 | 3.2 | 6.08 |
| | | 1.1 | | 2.64 | | 4.62 | | 6.82 |
| Mean | 0.2 | 0.8 | 0.7 | 1.7 | 1.3 | 2.6 | 4.1 | 5.1 |
| s.e.m. | 0.1 | 0.2 | 0.1 | 0.4 | 0.1 | 0.6 | 0.4 | 0.9 |

| | | | |
|---|---|---|---|
| t = 2.72, | t = 2.17, | t = 2.11, | t = 1.12, NS |
| p < 0.05 | p = 0.05 | p = 0.06 | |

| | 2 hr | | 4 hr | | 6 hr | | 24 hr | |
|---|---|---|---|---|---|---|---|---|
| | Water | | Water | | Water | | Water | |
| | Intake | | Intake | | Intake | | Intake | |
| | Comp. A | VEH | Comp. A | VEH | Comp. A | VEH | Comp. A | VEH |
| | 1 | 2 | 5 | 8 | 6 | 10 | 25 | 36 |
| | 5 | 1 | 8 | 3 | 7 | 7 | 22 | 17 |
| | 3 | 1 | 4 | 8 | 9 | 12 | 13 | 28 |
| | 0 | 1 | 5 | 9 | 5 | 4 | 16 | 20 |
| | 1 | 5 | 2 | 6 | 11 | 26 | 14 | 10 |
| | 1 | 6 | 0 | 2 | 10 | 23 | 28 | 18 |
| | | 1 | | 4 | | 8 | | 14 |
| Mean | 1.8 | 2.4 | 4 | 5.7 | 8 | 12.8 | 18.9 | 20.4 |
| s.e.m. | 0.8 | 0.7 | 1.1 | 1 | 1 | 3.2 | 2.3 | 3.3 |

| | | | | |
|---|---|---|---|---|
| t value | t = 0.54, NS | t = 1.12, NS | t = 1.47, NS | t = 0.39, NS |

What is claimed is:

1. A method for treating psychiatric disorders including but not limited to depression, generalized anxiety, social phobia, panic disorder, and sleep disorders comprising administration of a therapeutically effective amount of an ALDH-2 inhibitor to a mammal in need thereof.

2. The method of claim 1, wherein the ALDH-2 inhibitor is a compound of the formula: 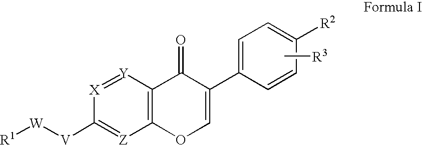
wherein:
R¹ is optionally substituted with from 1 to 3 substituents independently selected from the group consisting of alkyl, cycloalkyl, heterocyclyl, aryl, heteroaryl, halo, ═O, B(OH)₂, NO₂, CF₃, OCF₃, CN, OR²⁰, SR, N(R²⁰)₂, S(O)R, SO₂R², SO₂N(R²)₂, S(O)₃R², P(O)(OR²⁰)₂, SO₂NR²⁰COR²², SO₂NR²CO₂R²², SO₂NR²⁰CON(R²)₂, NR²⁰COR²², NR²CO₂R²², NR²⁰CON(R²⁰)₂, NR²⁰C(NR²⁰)NHR²², COR²⁰, CO 20, CON(R²⁰, C(O)N(R²⁰)₂, C(S)N(R²⁰)₂, C(O)NR²⁰SO₂R²², NR²⁰SO₂R²², SO₂NR²⁰COR²², OCONR²⁰SO₂R²², OC(O)R²⁰, C(O)OCH₂OC(O)R²⁰ and OCON(R²⁰)₂, and
further wherein each optional alkyl, cycloalkyl, heteroaryl, aryl, and heterocyclyl substituent is further optionally substituted with aryl, heteroaryl, halo, NO₂, alkyl, ═O, B(OH)₂, CF₃, OCF₃, Si(CH₃)₃, amino, mono- or di-alkylamino, alkyl or aryl or heteroaryl amide, NR²⁰COR²², NR²SO₂R²², COR²⁰, CO₂R²⁰, CON(R²⁰)₂, C(O)N(R²⁰)₂, C(S)N(R²⁰)₂, NR²⁰CON(R²⁰)₂, OC(O)R²⁰, OC(O)N(R²⁰)₂, S(O)₃R², P(O)(OR²⁰)₂, SR²⁰, S(O)R²², SO₂R²², SO₂N(R²⁰)₂, CN, or OR²;
R² is hydrogen, hydroxy, halogen, optionally substituted lower alkoxy, optionally substituted lower alkyl, cyano, optionally substituted heteroaryl, C(O)OR⁵, —C(O)R⁵, —SO₂R¹⁵, —B(OH)₂, —OP(O)(OR⁵)₂, C(NR²⁰)NHR²², —NHR⁴, or —C(O)NHR⁵, in which,
R⁴ is hydrogen, —C(O)NHR⁵, or —SO₂R¹⁵, or —C(O)R⁵;
R⁵ is hydrogen, optionally substituted lower alkyl;
R¹⁵ is optionally substituted lower alkyl or optionally substituted phenyl; or
R² is —O-Q-R⁶, in which Q is a covalent bond or lower alkylene and R⁶ is optionally substituted heteroaryl;
R³ is hydrogen, cyano, optionally substituted amino, lower alkyl, lower alkoxy, or halo;
X, Y and Z are chosen from —CR⁷— and —N—, in which R⁷ is hydrogen, lower alkyl, lower alkoxy, or halo;
V is oxygen, sulfur, or —NH—; and
W is -Q¹-T-Q²-, wherein
Q¹ is a covalent bond or C₁₋₆ linear or branched alkylene optionally substituted with hydroxy, lower alkoxy, amino, cyano, or ═O;
Q² is C₁₋₆ linear or branched alkylene optionally substituted with hydroxy, lower alkoxy, amino, cyano, or ═O; and
T is a covalent bond, —O—, or —NH—, or
T and Q¹ may together form a covalent bond,
R²⁰ and R²² are independently selected from the group consisting of hydrogen, hydroxy, C₁₋₁₅ alkyl, C₂₋₁₅ alkenyl, C₂₋₁₅ alkynyl, cycloalkyl, heterocyclyl, aryl, benzyl, and heteroaryl,
wherein the alkyl, alkenyl, alkynyl, cycloalkyl, heterocyclyl, aryl, benzyl, and heteroaryl moieties are optionally substituted with from 1 to 3 substituents independently selected from halo, alkyl, mono- or dialkylamino, alkyl or aryl or heteroaryl amide, CN, O—C₁₋₆ alkyl, CF₃, COOH, OCF₃, B(OH)₂, Si(CH₃)₃, aryl, and heteroaryl.

3. The method of claim 2, wherein X, Y, and Z are —CH—.

4. The method of claim 3 wherein,
R² and R³ are independently alkyl, amino, —B(OH)₂, —C(NR²⁰)NHR²², —C(O)NHR⁵, —C(O)R⁵, —C(O)OR⁵, cyano, hydrogen, halogen, lower alkoxy, —NHSO₂R¹⁵, hydroxy, —OP(O)(OR⁵)₂, or —SO₂R⁵.

5. The method of claim 4, wherein V is —O—.

6. The method of claim 5, wherein Q¹ and/or Q² is branch alkylene.

7. The method of claim 5, wherein Q¹ and T together form a covalent bond and Q² is methylene so that W is methylene.

8. The method of claim 7, wherein R² is hydroxy or —NHSO₂CH₃ and R³ is hydrogen.

9. The method of claim 7, where in R¹ is phenyl optionally substituted with one to three members selected from lower alkyl, B(OH)₂, C(O)N(R²⁰)₂, C(S)N(R²⁰)₂, CF₃, CN, CON(R²)₂, COOR²⁰, halogen, heteroaryl, OCF₃, OR²⁰, and phenyl optionally substituted with 1 to 3 members selected from CF₃, halogen, OR²⁰, CN, heteroaryl, C(O)OR²⁰, and lower alkyl.

10. The method of claim 7 selected from the group consisting of:
3-((3-(4-hydroxyphenyl)-4-oxo-4H-chromen-7-yloxy)methyl)benzoic acid;
methyl 3-((3-(4-hydroxyphenyl)-4-oxo-4H-chromen-7-yloxy)methyl)benzoate;
methyl 3-((3-(4-(methylsulfonamido)phenyl)-4-oxo-4H-chromen-7-yloxy)methyl)benzoate;
4-((3-(4-hydroxyphenyl)-4-oxo-4H-chromen-7-yloxy)methyl)phenyl propionate;
methyl 4-((3-(4-hydroxyphenyl)-4-oxo-4H-chromen-7-yloxy)methyl)benzoate;
4-((3-(4-hydroxyphenyl)-4-oxo-4H-chromen-7-yloxy)methyl)benzoic acid;
3-((3-(4-aminophenyl)-4-oxo-4H-chromen-7-yloxy)methyl)benzoic acid;
allyl 3-((3-(4-aminophenyl)-4-oxo-4H-chromen-7-yloxy)methyl)benzoate;
allyl 3-((3-(4-(methylsulfonamido)phenyl)-4-oxo-4H-chromen-7-yloxy)methyl)benzoate;
3-((3-(4-(methylsulfonamido)phenyl)-4-oxo-4H-chromen-7-yloxy)methyl)benzoic acid;
methyl 3-((3-(4-(2-amino-2-oxoethoxy)phenyl)-4-oxo-4H-chromen-7-yloxy)methyl)benzoate;
methyl 3-((3-(4-acetoxyphenyl)-4-oxo-4H-chromen-7-yloxy)methyl)benzoate;
isopropyl 3-((3-(4-hydroxyphenyl)-4-oxo-4H-chromen-7-yloxy)methyl)benzoate;
3-{[3-(4-hydroxyphenyl)-4-oxochromen-7-yloxy]methyl}benzenecarbonitrile;
3-(4-hydroxyphenyl)-7-[(3-(1H-1,2,3,4-tetrazol-5-yl)phenyl)methoxy]chromen-4-one;
3-(4-hydroxyphenyl)-7-{[3-(trifluoromethyl)phenyl]methoxy}chromen-4-one;
7-{[3-fluoro-5-(trifluoromethyl)phenyl]methoxy}-3-(4-hydroxyphenyl)chromen-4-one;
prop-2-enyl 3-{[3-(4-hydroxyphenyl)-4-oxochromen-7-yloxy]methyl}benzoate;
3-{[3-(4-aminophenyl)-4-oxochromen-7-yloxy]methyl}benzenecarbonitrile;
3-{[3-(4-hydroxyphenyl)-4-oxochromen-7-yloxy]methyl}benzamide;
3-[(3-{4-[(methylsulfonyl)amino]phenyl}-4-oxochromen-7-yloxy)methyl]benzamide;
3-[(3-{4-[(methylsulfonyl)amino]phenyl}-4-oxochromen-7-yloxy)methyl]benzenecarbonitrile;
methylethyl 3-[(3-{4-[(methylsulfonyl)amino]phenyl}-4-oxochromen-7-yloxy)methyl]benzoate;
3-{4-[(methylsulfonyl)amino]phenyl}-7-{[3-(trifluoromethyl)phenyl]methoxy}chromen-4-one;
3-{[3-(4-aminophenyl)-4-oxochromen-7-yloxy]methyl}benzamide;
3-(4-hydroxyphenyl)-7-{[3-(trifluoromethoxy)phenyl]methoxy}chromen-4-one;
3-(4-aminophenyl)-7-{[3-(trifluoromethyl)phenyl]methoxy}chromen-4-one;
3-(4-aminophenyl)-7-{[3-(trifluoromethoxy)phenyl]methoxy}chromen-4-one;
3-{4-[(methylsulfonyl)amino]phenyl}-7-{[3-(trifluoromethoxy)phenyl]methoxy}chromen-4-one;
methylethyl 3-{[3-(4-aminophenyl)-4-oxochromen-7-yloxy]methyl}benzoate;
methyl 3-({3-[4-(hydroxymethyl)phenyl]-4-oxochromen-7-yloxy}methyl)benzoate;
methyl 3-({3-[4-(dihydroxyboramethyl)phenyl]-4-oxochromen-7-yloxy}methyl)benzoate;
3,3-dimethyl-3-silabutyl 3-{[3-(4-carbamoylphenyl)-4-oxochromen-7-yloxy]methyl}benzoate;
3,3-dimethyl-3-silabutyl 3-[(3-{3-[(methylsulfonyl)amino]phenyl}-4-oxochromen-7-yloxy)methyl]benzoate;
3,3-dimethyl-3-silabutyl 3-{[3-(3-cyanophenyl)-4-oxochromen-7-yloxy]methyl}benzoate;
methyl 3-{[3-(4-{[(4-methylphenyl)sulfonyl]amino}phenyl)-4-oxochromen-7-yloxy]methyl}benzoate;
methyl 3-({3-[4-(amino(hydroxyimino)methyl)phenyl]-4-oxochromen-7-yloxy}methyl)benzoate;
3-[(2-hydroxy-3-{4-[(methylsulfonyl)amino]phenyl}-4-oxochroman-7-yloxy)methyl]benzoic acid;
3-{[3-(4-carbamoylphenyl)-4-oxochromen-7-yloxy]methyl}benzoic acid, methanol, methanol;
3-({3-[4-(methylsulfonyl)phenyl]-4-oxochromen-7-yloxy}methyl)benzoic acid;
4-((3-(4-hydroxyphenyl)-4-oxo-4H-chromen-7-yloxy)methyl)phenylboronic acid;
3-((3-(4-hydroxyphenyl)-4-oxo-4H-chromen-7-yloxy)methyl)phenylboronic acid;
N-{[4-chloro-3-(trifluoromethyl)phenyl]methyl}(3-{[3-(4-hydroxyphenyl)-4-oxochromen-7-yloxy]methyl}phenyl)carboxamide;
(3-{[3-(4-hydroxyphenyl)-4-oxochromen-7-yloxy]methyl}phenyl)-N-{[3-(trifluoromethyl)phenyl]methyl}carboxamide;
methylethyl 3-{[3-(4-carbamoylphenyl)-4-oxochromen-7-yloxy]methyl}benzoate;
2-{[3-(4-hydroxyphenyl)-4-oxochromen-7-yloxy]methyl}benzenecarbonitrile; and
4-{[3-(4-hydroxyphenyl)-4-oxochromen-7-yloxy]methyl}benzenecarbonitrile.

11. The method of claim 7, where in R¹ is heteroaryl optionally substituted with one to three members selected from lower alkyl, B(OH)₂, C(O)N(R²⁰)₂, C(S)N(R²⁰)₂, CF₃, CN, CON(R²⁰)₂, COOR²⁰, halogen, heteroaryl, OCF₃, OR²⁰, and phenyl optionally substituted with 1 to 3 members selected from CF₃, halogen, OR²⁰, CN, heteroaryl, C(O)OR²⁰, and lower alkyl.

12. The method of claim 11, wherein R¹ is selected from the group consisting of oxadiazole, oxazole, pyrazole, thiazole, pyridine, indolizinyl, benzothiazolyl, benzothienyl, thiadiazole, pyrrole, imidazole, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, thiazole, isothiazole, phenazine, oxazole, isoxazole, phenoxazine, phenothiazine, imidazolidine, and imidazoline.

13. The method of claim 12, wherein R¹ is optionally substituted oxadiazole.

14. The method of claim 13, selected from the group consisting of:
3-(4-hydroxyphenyl)-7-((5-(3-(trifluoromethyl)phenyl)-1,2,4-oxadiazol-3-yl)methoxy)-4H-chromen-4-one;
3-(4-hydroxyphenyl)-7-((5-phenyl-1,2,4-oxadiazol-3-yl)methoxy)-4H-chromen-4-one;
3-(4-hydroxyphenyl)-6-((3-(3-(trifluoromethyl)phenyl)-1,2,4-oxadiazol-5-yl)methoxy)-4H-chromen-4-one;
3-(4-hydroxyphenyl)-7-((3-phenyl-1,2,4-oxadiazol-5-yl)methoxy)-4H-chromen-4-one;
N-(4-(4-oxo-7-((5-(3-(trifluoromethyl)phenyl)-1,2,4-oxadiazol-3-yl)methoxy)-4H-chromen-3-yl)phenyl)methanesulfonamide;
3-(4-aminophenyl)-7-((5-(3-(trifluoromethyl)phenyl)-1,2,4-oxadiazol-3-yl)methoxy)-4H-chromen-4-one;
7-((5-(3-fluoro-5-(trifluoromethyl)phenyl)-1,2,4-oxadiazol-3-yl)methoxy)-3-(4-hydroxyphenyl)-4H-chromen-4-one;
3-(4-aminophenyl)-7-((5-(3-fluoro-5-(trifluoromethyl)phenyl)-1,2,4-oxadiazol-3-yl)methoxy)-4H-chromen-4-one;
N-(4-(7-((5-(3-fluoro-5-(trifluoromethyl)phenyl)-1,2,4-oxadiazol-3-yl)methoxy)-4-oxo-4H-chromen-3-yl)phenyl)methanesulfonamide;
3-(4-hydroxyphenyl)-7-((5-(4-methoxy-3-(trifluoromethyl)phenyl)-1,2,4-oxadiazol-3-yl)methoxy)-4H-chromen-4-one;
7-((5-(4-fluoro-3-(trifluoromethyl)phenyl)-1,2,4-oxadiazol-3-yl)methoxy)-3-(4-hydroxyphenyl)-4H-chromen-4-one;
7-((5-(2,5-bis(trifluoromethyl)phenyl)-1,2,4-oxadiazol-3-yl)methoxy)-3-(4-hydroxyphenyl)-4H-chromen-4-one;
7-({5-[3,5-bis(trifluoromethyl)phenyl](1,2,4-oxadiazol-3-yl)}methoxy)-3-(4-hydroxyphenyl)chromen-4-one;
3-(4-methoxyphenyl)-7-{[5-(2-methoxyphenyl)(1,2,4-oxadiazol-3-yl)]methoxy}chromen-4-one;
3-(4-hydroxyphenyl)-2-(trifluoromethyl)-6-({5-[3-(trifluoromethyl)phenyl](1,2,4-oxadiazol-3-yl)}methoxy)chromen-4-one;
3-(4-hydroxyphenyl)-6-({5-[4-methoxy-3-(trifluoromethyl)phenyl](1,2,4-oxadiazol-3-yl)}methoxy)-2-(trifluoromethyl)chromen-4-one;
6-({5-[3-fluoro-5-(trifluoromethyl)phenyl](1,2,4-oxadiazol-3-yl)}methoxy)-3-(4-hydroxyphenyl)-2-(trifluoromethyl)chromen-4-one;
3-(3-{[3-(4-hydroxyphenyl)-4-oxochromen-7-yloxy]methyl}-1,2,4-oxadiazol-5-yl)benzenecarbonitrile;
3-(4-hydroxyphenyl)-7-{[5-(3-(1H-1,2,3,4-tetraazol-5-yl)phenyl)(1,2,4-oxadiazol-3-yl)]methoxy}chromen-4-one;
3-(3-{[3-(4-hydroxyphenyl)-4-oxochromen-7-yloxy]methyl}-1,2,4-oxadiazol-5-yl)benzoic acid;
prop-2-enyl 3-(3-{[3-(4-hydroxyphenyl)-4-oxochromen-7-yloxy]methyl}-1,2,4-oxadiazol-5-yl)benzoate;
7-{[5-(3-fluorophenyl)(1,2,4-oxadiazol-3-yl)]methoxy}-3-(4-hydroxyphenyl)chromen-4-one;
methylethyl 3-(3-{[3-(4-hydroxyphenyl)-4-oxochromen-7-yloxy]methyl}-1,2,4-oxadiazol-5-yl)benzoate;
7-{[5-(3-fluorophenyl)(1,3,4-oxadiazol-2-yl)]methoxy}-3-(4-hydroxyphenyl)chromen-4-one;
3-(4-hydroxyphenyl)-7-({5-[3-(trifluoromethyl)phenyl](1,3,4-oxadiazol-2-yl)}methoxy)chromen-4-one;
3-[7-({5-[5-fluoro-3-(trifluoromethyl)phenyl](1,2,4-oxadiazol-3-yl)}methoxy)-4-oxochromen-3-yl]benzenecarbonitrile;
7-({5-[5-fluoro-3-(trifluoromethyl)phenyl](1,2,4-oxadiazol-3-yl)}methoxy)-3-{3-[(methylsulfonyl)amino]phenyl}chromen-4-one;
7-({5-[5-fluoro-3-(trifluoromethyl)phenyl](1,2,4-oxadiazol-3-yl)}methoxy)-3-[4-(methylsulfonyl)phenyl]chromen-4-one;
4-[7-({5-[5-fluoro-3-(trifluoromethyl)phenyl](1,2,4-oxadiazol-3-yl)}methoxy)-4-oxochromen-3-yl]benzenecarbonitrile;
4-[7-({5-[5-fluoro-3-(trifluoromethyl)phenyl](1,2,4-oxadiazol-3-yl)}methoxy)-4-oxochromen-3-yl]benzamide;
3-(3-acetylphenyl)-7-({5-[5-fluoro-3-(trifluoromethyl)phenyl] (1,2,4-oxadiazol-3-yl)}methoxy)chromen-4-one;
7-({5-[3-fluoro-5-(trifluoromethyl)phenyl](1,3,4-oxadiazol-2-yl)}methoxy)-3-(4-hydroxyphenyl)chromen-4-one;
methyl 3-[7-({5-[5-fluoro-3-(trifluoromethyl)phenyl](1,2,4-oxadiazol-3-yl)}methoxy)-4-oxochromen-3-yl]benzoate;
7-({3-[5-fluoro-3-(trifluoromethyl)phenyl](1,2,4-oxadiazol-5-yl)}ethoxy)-3-(4-hydroxyphenyl)chromen-4-one;
3-(4-fluorophenyl)-7-{[5-(2-methoxyphenyl)(1,2,4-oxadiazol-3-yl)]methoxy}chromen-4-one;
3-(4-methylphenyl)-7-({5-[3-(trifluoromethyl)phenyl](1,2,4-oxadiazol-3-yl)}methoxy)chromen-4-one;
4-[4-oxo-7-({5-[3-(trifluoromethyl)phenyl](1,2,4-oxadiazol-3-yl)}methoxy)chromen-3-yl]benzenecarbonitrile;
3-(3-fluorophenyl)-7-({5-[3-(trifluoromethyl)phenyl](1,2,4-oxadiazol-3-yl)}methoxy)chromen-4-one;
7-({5-[5-fluoro-3-(trifluoromethyl)phenyl](1,2,4-oxadiazol-3-yl)}ethoxy)-3-(4-hydroxyphenyl)chromen-4-one;
7-({5-[5-fluoro-3-(trifluoromethyl)phenyl](1,2,4-oxadiazol-3-yl)}methoxy)-3-(4-{[(4-methylphenyl)sulfonyl]amino}phenyl)chromen-4-one;
7-{[5-(3-chlorophenyl)(1,3,4-oxadiazol-2-yl)]methoxy}-3-(4-hydroxyphenyl)chromen-4-one;
7-{[5-(4-fluorophenyl)(1,3,4-oxadiazol-2-yl)]methoxy}-3-(4-hydroxyphenyl)chromen-4-one;
7-({5-[5-fluoro-3-(trifluoromethyl)phenyl](1,2,4-oxadiazol-3-yl)}methoxy)-3-[4-(hydroxymethyl)phenyl]chromen-4-one;
4-[7-({5-[3-fluoro-5-(trifluoromethyl)phenyl](1,2,4-oxadiazol-3-yl)}methoxy)-4-oxochromen-3-yl]benzoic acid;
2-fluoro-5-[7-({5-[5-fluoro-3-(trifluoromethyl)phenyl](1,2,4-oxadiazol-3-yl)}methoxy)-4-oxochromen-3-yl]benzenecarbonitrile;
7-({3-[2-fluoro-4-(trifluoromethyl)phenyl](1,2,4-oxadiazol-5-yl)}ethoxy)-3-{4-[(methylsulfonyl)amino]phenyl}chromen-4-one;
3-[4-(dihydroxyboramethyl)phenyl]-7-({3-[2-fluoro-4-(trifluoromethyl)phenyl](1,2,4-oxadiazol-5-yl)}ethoxy)chromen-4-one;
7-{[5-(4-fluorophenyl)(1,2,4-oxadiazol-3-yl)]methoxy}-3-(4-hydroxyphenyl)chromen-4-one;
7-{[5-(4-fluorophenyl)(1,2,4-oxadiazol-3-yl)]methoxy}-3-{4-[(methylsulfonyl)amino]phenyl}chromen-4-one;
3-(4-hydroxyphenyl)-7-[(5-(3-pyridyl)(1,3,4-oxadiazol-2-yl))methoxy]chromen-4-one;
7-{[5-(4-fluorophenyl)(1,3,4-oxadiazol-2-yl)]ethoxy}-3-(4-hydroxyphenyl)chromen-4-one;
3-(4-hydroxyphenyl)-7-[(5-(3-pyridyl)(1,3,4-oxadiazol-2-yl))ethoxy]chromen-4-one;
3-(4-hydroxyphenyl)-7-[(3-(3-pyridyl)(1,2,4-oxadiazol-5-yl))methoxy]chromen-4-one;
3-(4-hydroxyphenyl)-7-({3-[3-(trifluoromethyl)phenyl] (1,2,4-oxadiazol-5-yl)}methoxy)chromen-4-one;
7-({3-[3-(trifluoromethyl)phenyl](1,2,4-oxadiazol-5-yl)}methoxy)-3-[4-({3-[3-(trifluoromethyl)phenyl](1,2,4-oxadiazol-5-yl)}methoxy)phenyl]chromen-4-one;
3-(4-hydroxyphenyl)-7-[(5-(4-pyridyl)(1,3,4-oxadiazol-2-yl))ethoxy]chromen-4-one;
3-[4-(dihydroxyboramethyl)phenyl]-7-{[5-(4-fluorophenyl)(1,2,4-oxadiazol-3-yl)]methoxy}chromen-4-one;
3-(4-hydroxyphenyl)-7-[(5-(4-pyridyl)(1,2,4-oxadiazol-3-yl))ethoxy]chromen-4-one;
7-{[5-(4-fluorophenyl)(1,3,4-oxadiazol-2-yl)]methoxy}-3-{4-[(methylsulfonyl)amino]phenyl}chromen-4-one;
7-{[3-(3-aminophenyl)(1,2,4-oxadiazol-5-yl)]methoxy}-3-(4-hydroxyphenyl)chromen-4-one;
3-(4-hydroxyphenyl)-7-[(5-(3-pyridyl)(1,2,4-oxadiazol-3-yl))ethoxy]chromen-4-one;
3-(4-hydroxyphenyl)-7-[(5-(3-pyridyl)(1,2,4-oxadiazol-3-yl))methoxy]chromen-4-one;
3-(4-hydroxyphenyl)-7-[(5-(2-pyridyl)(1,2,4-oxadiazol-3-yl))methoxy]chromen-4-one;
7-{[3-(5-bromo(3-pyridyl))(1,2,4-oxadiazol-5-yl)]ethoxy}-3-(4-hydroxyphenyl)chromen-4-one;
3-(4-hydroxyphenyl)-7-[(5-(2-pyridyl)(1,2,4-oxadiazol-3-yl))ethoxy]chromen-4-one;
7-((1R)-1-{3-[3-fluoro-5-(trifluoromethyl)phenyl](1,2,4-oxadiazol-5-yl)}ethoxy)-3-(4-hydroxyphenyl)chromen-4-one;
7-({3-[3-fluoro-5-(trifluoromethyl)phenyl](1,2,4-oxadiazol-5-yl)}methoxy)-3-(4-hydroxyphenyl)chromen-4-one;
7-((1S)-1-{3-[3-fluoro-5-(trifluoromethyl)phenyl](1,2,4-oxadiazol-5-yl)}ethoxy)-3-(4-hydroxyphenyl)chromen-4-one;
7-((1R)-1-{3-[3-fluoro-5-(trifluoromethyl)phenyl](1,2,4-oxadiazol-5-yl)}ethoxy)-3-(4-hydroxyphenyl)chromen-4-one;
7-(1-{3-[3-fluoro-5-(trifluoromethyl)phenyl](1,2,4-oxadiazol-5-yl)}-isopropoxy)-3-(4-hydroxyphenyl)chromen-4-one;
3-(4-hydroxyphenyl)-7-{[5-(2-methoxyphenyl)(1,3,4-oxadiazol-2-yl)]methoxy}chromen-4-one;
3-(4-hydroxyphenyl)-7-{[5-(4-methoxyphenyl)(1,3,4-oxadiazol-2-yl)]methoxy}chromen-4-one;
3-(4-hydroxyphenyl)-7-{[5-(2-methylphenyl)(1,3,4-oxadiazol-2-yl)]methoxy}chromen-4-one;
3-(4-hydroxyphenyl)-7-{[5-(3-methylphenyl)(1,3,4-oxadiazol-2-yl)]methoxy}chromen-4-one;
3-(4-hydroxyphenyl)-7-{[5-(4-methylphenyl)(1,3,4-oxadiazol-2-yl)]methoxy}chromen-4-one;
3-(4-hydroxyphenyl)-7-({5-[4-(trifluoromethyl)phenyl](1,3,4-oxadiazol-2-yl)}methoxy)chromen-4-one;
3-(4-hydroxyphenyl)-7-{[5-(4-methoxyphenyl)(1,2,4-oxadiazol-3-yl)]methoxy}chromen-4-one;
3-(4-hydroxyphenyl)-7-{[5-(3-methoxyphenyl)(1,2,4-oxadiazol-3-yl)]methoxy}chromen-4-one;
7-{[5-(4-chlorophenyl)(1,3,4-oxadiazol-2-yl)]methoxy}-3-(4-hydroxyphenyl)chromen-4-one;
7-{[5-(2-chlorophenyl)(1,3,4-oxadiazol-2-yl)]methoxy}-3-(4-hydroxyphenyl)chromen-4-one; and
3-(4-hydroxyphenyl)-7-{[5-(3-methoxyphenyl)(1,3,4-oxadiazol-2-yl)]methoxy}chromen-4-one.

15. The method of claim 12, wherein R¹ is optionally substituted oxazole.

16. The method of claim 15, selected from the group consisting of:
3-(4-hydroxyphenyl)-7-[(2-phenyl(1,3-oxazol-4-yl))methoxy]chromen-4-one;
7-({5-[3,5-bis(trifluoromethyl)phenyl]isoxazol-3-yl}methoxy)-3-(4-hydroxyphenyl)chromen-4-one;
3-{4-[(methylsulfonyl)amino]phenyl}-7-[(2-phenyl(1,3-oxazol-4-yl))methoxy]chromen-4-one;
3-(4-hydroxyphenyl)-7-({5-[3-(trifluoromethyl)phenyl]isoxazol-3-yl}methoxy)chromen-4-one;
3-(4-hydroxyphenyl)-7-({3-[3-(trifluoromethyl)phenyl]isoxazol-5-yl}methoxy)chromen-4-one;
3-(4-hydroxyphenyl)-7-[(5-phenylisoxazol-3-yl)methoxy]chromen-4-one;
3-(4-hydroxyphenyl)-7-({2-[3-(trifluoromethyl)phenyl](1,3-oxazol-4-yl)}methoxy)chromen-4-one;
7-{[5-(4-chlorophenyl)isoxazol-3-yl]methoxy}-3-(4-hydroxyphenyl)chromen-4-one;
7-{[5-(3,4-dichlorophenyl)isoxazol-3-yl]methoxy}-3-(4-hydroxyphenyl)chromen-4-one;
7-({2-[5-fluoro-3-(trifluoromethyl)phenyl](1,3-oxazol-4-yl)}methoxy)-3-(4-hydroxyphenyl)chromen-4-one;
methyl 2-{[3-(4-hydroxyphenyl)-4-oxochromen-7-yloxy]methyl}-1,3-oxazole-4-carboxylate;
2-{[3-(4-hydroxyphenyl)-4-oxochromen-7-yloxy]methyl}-1,3-oxazole-4-carboxylic acid;
(2-{[3-(4-hydroxyphenyl)-4-oxochromen-7-yloxy]methyl}(1,3-oxazol-4-yl))-N-methylcarboxamide;
7-{[2-(4-chlorophenyl)(1,3-oxazol-4-yl)]methoxy}-3-(4-hydroxyphenyl)chromen-4-one;
7-{[2-(3,4-difluorophenyl)(1,3-oxazol-4-yl)]methoxy}-3-(4-hydroxyphenyl)chromen-4-one;
7-{[2-(3,5-difluorophenyl)(1,3-oxazol-4-yl)]methoxy}-3-(4-hydroxyphenyl)chromen-4-one;
7-{[2-(4-fluorophenyl)(1,3-oxazol-4-yl)]methoxy}-3-(4-hydroxyphenyl)chromen-4-one;
3-(4-hydroxyphenyl)-7-{[2-(3,4,5-trifluorophenyl)(1,3-oxazol-4-yl)]methoxy}chromen-4-one;
3-(4-hydroxyphenyl)-7-[(5-(2-pyridyl)isoxazol-3-yl)methoxy]chromen-4-one;
7-(3-{2-[3-fluoro-5-(trifluoromethyl)phenyl] (1,3-oxazol-4-yl)}propoxy)-3-(4-hydroxyphenyl)chromen-4-one; and
4-[7-({2-[5-fluoro-3-(trifluoromethyl)phenyl](1,3-oxazol-4-yl)}methoxy)-4-oxochromen-3-yl]phenyl dihydrogen phosphate.

17. The method of claim 12, wherein R¹ is optionally substituted thiazole.

18. The method of claim 17, selected from the group consisting of:
3-{4-[(methylsulfonyl)amino]phenyl}-7-({4-methyl-2-[4-(trifluoromethyl)phenyl](1,3-thiazol-5-yl)}methoxy)chromen-4-one;
ethyl 4-[7-({4-methyl-2-[4-(trifluoromethyl)phenyl](1,3-thiazol-5-yl)}methoxy)-4-oxochromen-3-yl]benzoate;
methyl 3-[7-({4-methyl-2-[4-(trifluoromethyl)phenyl](1,3-thiazol-5-yl)}methoxy)-4-oxochromen-3-yl]benzoate;
3-(4-hydroxyphenyl)-7-({4-methyl-2-[4-(trifluoromethyl)phenyl] (1,3-thiazol-5-yl)}methoxy)chromen-4-one;
3-(4-{[(4-methylphenyl)sulfonyl]amino}phenyl)-7-({4-methyl-2-[4-(trifluoromethyl)phenyl](1,3-thiazol-5-yl)}methoxy)chromen-4-one; and
3-{4-[(methylsulfonyl)amino]phenyl}-7-({2-[4-(trifluoromethyl)phenyl](1,3-thiazol-4-yl)}methoxy)chromen-4-one.

19. The method of claim 12, wherein R¹ is optionally substituted pyridine.

20. The method of claim 19, selected from the group consisting of:
3-(4-hydroxyphenyl)-7-(4-pyridylmethoxy)chromen-4-one;
3-(4-hydroxyphenyl)-7-(2-pyridylmethoxy)chromen-4-one;
3-(4-hydroxyphenyl)-7-{[6-(trifluoromethyl)(3-pyridyl)]methoxy}chromen-4-one;
7-{[6-(trifluoromethyl)(3-pyridyl)]methoxy}-3-(4-{[6-(trifluoromethyl)(3-pyridyl)]methoxy}phenyl)chromen-4-one;
3-(4-hydroxyphenyl)-7-[(6-pyrazolyl(3-pyridyl))methoxy]chromen-4-one; and
3-(4-hydroxyphenyl)-7-(3-pyridylmethoxy)chromen-4-one.

21. The method of claim 12, wherein R¹ is optionally substituted pyrazole.

22. The method of claim 21, selected from the group consisting of:
7-{2-[4-(4-chlorophenyl)pyrazolyl]ethoxy}-3-(4-hydroxyphenyl)chromen-4-one;
ethyl 1-{2-[3-(4-hydroxyphenyl)-4-oxochromen-7-yloxy]ethyl}pyrazole-4-carboxylate; and
3-(4-hydroxyphenyl)-7-{2-[3-(trifluoromethyl)pyrazolyl]ethoxy}chromen-4-one.

23. The method of claim 7, where in R¹ is heterocycyl optionally substituted with one to three members selected from lower alkyl, B(OH)₂, C(O)N(R²⁰)₂, C(S)N(R²⁰)₂, CF₃, CN, CON(R²⁰)₂, COOR²⁰, halogen, heteroaryl, OCF₃, OR²⁰, and phenyl optionally substituted with 1 to 3 members selected from CF₃, halogen, OR²⁰, CN, heteroaryl, C(O)OR²⁰, and lower alkyl.

24. The method of claim 14, wherein R¹ is selected from the group consisting of tetrahydrofuranyl, morpholino, oxathiane, thiomorpholino, tetraydropthiophenyl, tetrahydropyranyl, tetrahydrothiopyranyl, piperidinyl, triazolidino, piperazinyl, dihydropyridino, pyrrolidinyl, imidazolidino, hexahydropyrimidine, hezahydropyridazine, and imidazoline.

25. The method of claim 24, wherein R¹ is piperazinyl.

26. The method of claim 25, selected from the group consisting of:
3-(4-hydroxyphenyl)-7-{2-[4-(4-methoxyphenyl)piperazinyl]ethoxy}chromen-4-one;
7-{2-[4-(4-fluorophenyl)piperazinyl]ethoxy}-3-(4-hydroxyphenyl)chromen-4-one;
7-{2-[4-(3-chlorophenyl)piperazinyl]ethoxy}-3-(4-hydroxyphenyl)chromen-4-one;
3-(4-hydroxyphenyl)-7-(2-{4-[3-(trifluoromethyl)phenyl]piperazinyl}ethoxy)chromen-4-one;
3-(4-hydroxyphenyl)-7-(2-piperazinylethoxy)chromen-4-one;
N-(3-fluorophenyl)(4-{2-[3-(4-hydroxyphenyl)-4-oxochromen-7-yloxy]ethyl}piperazinyl)carboxamide;
7-[2-(4-{[(4-fluorophenyl)amino]thioxomethyl}piperazinyl)ethoxy]-3-(4-hydroxyphenyl)chromen-4-one;
N-(2,4-difluorophenyl)(4-{2-[3-(4-hydroxyphenyl)-4-oxochromen-7-yloxy]ethyl}piperazinyl)carboxamide;
7-{2-[4-(2-fluorophenyl)piperazinyl]ethoxy}-3-(4-hydroxyphenyl)chromen-4-one;
3-(4-hydroxyphenyl)-7-{2-[4-(2-methylphenyl)piperazinyl]ethoxy}chromen-4-one;
3-(4-hydroxyphenyl)-7-{2-[4-(2-methoxyphenyl)piperazinyl]ethoxy}chromen-4-one;
7-{2-[4-(3-fluorophenyl)piperazinyl]ethoxy}-3-(4-hydroxyphenyl)chromen-4-one;
3-(4-hydroxyphenyl)-7-{2-[4-(3-methylphenyl)piperazinyl]ethoxy}chromen-4-one;
3-(4-hydroxyphenyl)-7-{2-[4-(3-methoxyphenyl)piperazinyl]ethoxy}chromen-4-one;
3-(4-hydroxyphenyl)-7-(2-{4-[4-(trifluoromethyl)phenyl]piperazinyl}ethoxy)chromen-4-one;
3-(4-hydroxyphenyl)-7-{2-[4-(4-methylphenyl)piperazinyl]ethoxy}chromen-4-one; and
3-(4-hydroxyphenyl)-7-(2-{4-[2-(trifluoromethyl)phenyl]piperazinyl}ethoxy)chromen-4-one.

27. The method of claim 5, wherein R³ is hydrogen.

28. The method of claim 27, where in R¹ is phenyl optionally substituted with one to three members selected from lower alkyl, B(OH)₂, C(O)N(R²⁰)₂, C(S)N(R²⁰)₂, CF₃, CN, CON(R²⁰)₂, COOR²⁰, halogen, heteroaryl, OCF₃, OR²⁰, and phenyl optionally substituted with 1 to 3 members selected from CF₃, halogen, OR²⁰, CN, heteroaryl, C(O)OR²⁰, and lower alkyl.

29. The method of claim 28, wherein T is a covalent bond.

30. The method of claim 29 selected from the group consisting of:
7-[2-(4-fluorophenyl)-2-oxoethoxy]-3-(4-methoxyphenyl)chromen-4-one;
7-[2-(4-fluorophenyl)-2-oxoethoxy]-3-(4-hydroxyphenyl)chromen-4-one;
3-(4-hydroxyphenyl)-7-{[3-(trifluoromethyl)phenyl]ethoxy}chromen-4-one;
7-[2-(3-fluorophenyl)-2-oxoethoxy]-3-(4-hydroxyphenyl)chromen-4-one;
3-(4-hydroxyphenyl)-7-{2-oxo-2-[4-(trifluoromethyl)phenyl]ethoxy}chromen-4-one;
3-(4-hydroxyphenyl)-7-{2-oxo-2-[2-(trifluoromethyl)phenyl]ethoxy}chromen-4-one;
3-(4-hydroxyphenyl)-7-(2-phenylethoxy)chromen-4-one;
3-(4-hydroxyphenyl)-7-(3-phenylpropoxy)chromen-4-one;
7-((2S)-2-hydroxy-3-phenylpropoxy)-3-(4-hydroxyphenyl)chromen-4-one; and
7-[2-(4-fluorophenyl)ethoxy]-3-(4-hydroxyphenyl)chromen-4-one.

31. The method of claim 28, wherein T is —NH— or —O—.

32. The method of claim 31 selected from the group consisting of:
2-[3-(4-hydroxyphenyl)-4-oxochromen-7-yloxy]-n-[3-(trifluoromethyl)phenyl]acetamide;
7-(3-{[(3,5-difluorophenyl)methyl]amino}-2-hydroxypropoxy)-3-(4-methoxyphenyl)chromen-4-one;
7-[(2S)-2-hydroxy-3-({[3-(trifluoromethyl)phenyl]methyl}amino)propoxy]-3-(4-methoxyphenyl)chromen-4-one;
2-[3-(4-hydroxyphenyl)-4-oxochromen-7-yloxy]-N-[2-(trifluoromethyl)phenyl]acetamide;
N-[(1S)-1-(4-fluorophenyl)ethyl]-2-[3-(4-hydroxyphenyl)-4-oxochromen-7-yloxy]acetamide;
N-(3-fluorophenyl)-2-[3-(4-hydroxyphenyl)-4-oxochromen-7-yloxy]acetamide;
7-[(2S)-2-hydroxy-3-({[3-(trifluoromethyl)phenyl]methyl}amino)propoxy]-3-(4-hydroxyphenyl)chromen-4-one;
3-(4-hydroxyphenyl)-7-[2-({[3-(trifluoromethyl)phenyl]methyl}amino)ethoxy]chromen-4-one;
7-((2S)-3-{[(3,5-difluorophenyl)methyl]amino}-2-hydroxypropoxy)-3-(4-hydroxyphenyl)chromen-4-one;
N-[(1R)-1-(4-fluorophenyl)ethyl]-2-[3-(4-hydroxyphenyl)-4-oxochromen-7-yloxy]acetamide;
7-(2-{[(4-fluorophenyl)ethyl]amino}ethoxy)-3-(4-hydroxyphenyl)chromen-4-one; and
7-[2-(4-chlorophenoxy)ethoxy]-3-(4-hydroxyphenyl)chromen-4-one.

33. The method of claim 1, wherein the ALDH-2 is 3-[(3-{4-[(methylsulfonyl)amino]phenyl}-4-oxochromen-7-yloxy)methyl]benzoic acid.

34. The method of claim 1, wherein the psychiatric disorder is anxiety.

35. The method of claim 1, wherein the psychiatric disorder is depression.
